SEQUENCE LISTING (2 sequences)

SEQ ID NO: 1 - RNA, 1796 nt, synthetic construct
  source 1..1796: mol_type = other RNA
agggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgtgggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcgg

SEQ ID NO: 2 - RNA, 730 nt, synthetic construct
  source 1..730: mol_type = other RNA
agggaaataagagagaaaagaagagtaagaagaaatataagagccaccatgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtgataagctgccttctgcggggcttgccttctggccatgcccttcttctctcccttgcacctgtacctcttggtctttgaataaagcctgagtaggaaggcgg

(19) United States
(12) Patent Application Publication
(10) Pub. No.: US 2025/0346624 A1
(43) Pub. Date: Nov. 13, 2025

(54) POLYNUCLEOTIDE AND PRODUCING METHOD THEREOF

(71) Applicant: Eisai R&D Management Co., Ltd., Tokyo (JP)

(72) Inventors: Kosuke NAKAMOTO, Ibaraki (JP); Yuta SUZUKI, Ibaraki (JP); Hiroki MUTO, Tsuchiura-shi (JP); Jung Hwa LEE, Andover, MA (US); Hyeong-Wook CHOI, Andover, MA (US)

(21) Appl. No.: 19/272,275

(22) Filed: Jul. 17, 2025

Related U.S. Application Data
(63) Continuation of application No. PCT/JP2025/007821, filed on Mar. 5, 2025.
(60) Provisional application No. 63/562,424, filed on Mar. 7, 2024.

(51) Int. Cl. C07H 21/02; C12N 9/00; C12N 9/12

(52) U.S. Cl. CPC C07H 21/02; C12N 9/1241; C12N 9/93; C12Y 207/07019; C12Y 605/01003

(57) ABSTRACT

Disclosed are a polynucleotide with improved stability in vivo, a producing method thereof, and a method of improving the stability of polynucleotide in vivo.

### CROSS-REFERENCE TO RELATED APPLICATIONS

This application is a continuation application of PCT Application No. PCT/JP2025/007821, filed on Mar. 5, 2025, which claims the benefit of priority from U.S. Provisional Patent Application No. 63/562,424, filed on Mar. 7, 2024. The entire contents of the above listed PCT and priority applications are incorporated herein by reference.

### SEQUENCE LISTING INCORPORATED BY REFERENCE

This application contains an ST.26 compliant Sequence Listing, which is submitted concurrently in xml format via Patent Center and is hereby incorporated by reference in its entirety. The .xml copy, created on Jun. 23, 2025, is named FP24-1078-00US-ECL_seql.xml and is 5,288 bytes in size.

### TECHNICAL FIELD

The present disclosure generally relates to a polynucleotide and a producing method thereof.

### BACKGROUND

As nucleic acid drugs, antisense oligonucleotides, decoy nucleic acids, ribozymes, aptamers, siRNA, miRNA, messenger RNA (mRNA), and the like are known. Among them, nucleic acid drugs containing mRNA can be used for prevention and treatment of diseases by causing the biosynthesis of the protein encoded by the mRNA in vivo (in a body) of a subject to be administered.

Since nucleic acids such as mRNA are easily degraded by nucleases and the like in living organisms, and their stability in vivo is low, there is a problem that the concentration of mRNA administered to a subject rapidly decreases in vivo. Such a rapid decrease in the concentration of mRNA in vivo can cause a reduction in medical efficacy or its duration. Therefore, research aimed at improving the stability of nucleic acids such as mRNA in vivo is being conducted.

Regarding an improvement of the stability of nucleic acids such as mRNA in vivo, for example, Patent Literature 1 (US 2018/0256750) discloses an invention characterized by a polynucleotide that encodes a polypeptide containing a 3′-stabilizing region and has improved stability compared to a wild-type polynucleotide. Additionally, for example, Non-Patent Literature 1 (Abhishek Aditham et al., “Chemically Modified mocRNAs for Highly Efficient Protein Expression in Mammalian Cells” ACS Chemical Biology 17, 12, 3352-3366 (2022)) discloses an approach aimed at enabling site-specific, robust, and modular chemical modification for high-efficiency and stable protein expression. This approach involves binding a synthetic oligonucleotide with a specific structure to the 3′-end of mRNA that comprises a template-encoded poly(A) tail.

### SUMMARY

The present disclosure relates to a polynucleotide with improved stability in vivo (in a body). Furthermore, the present disclosure also relates to a producing method of such a polynucleotide and a method of improving the stability of a polynucleotide in vivo.

The present inventors have found that the stability of a polynucleotide such as mRNA in vivo can be enhanced by introducing an oligonucleotide or its derivative with a specific structure to the 3′-end of the polynucleotide. The present disclosure relates, for example, to the following:

[1] A polynucleotide represented by the following formula (I):

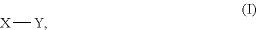

wherein Y is represented by the following formula (II-A):

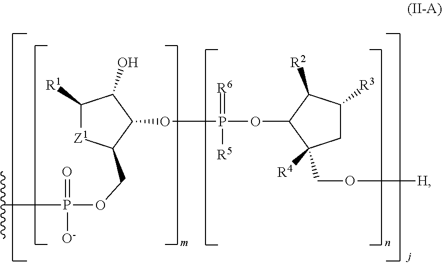

the following formula (III):

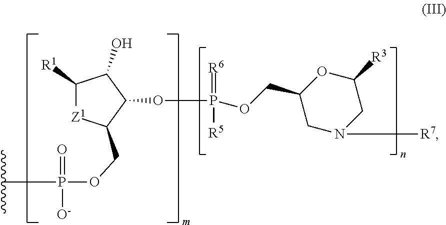

the following formula (IV):

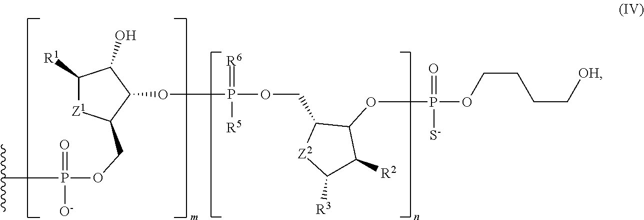

or the following formula (V):

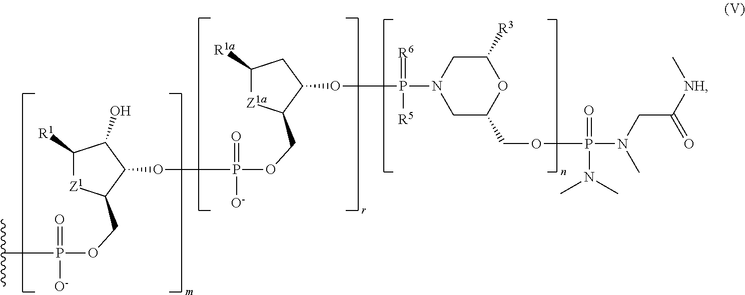

wherein
the wavy line represents a bond to X,
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II-A) or formula (IV),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
Z¹, Z^{1a}, and Z² each independently represent O, S, Se, NR¹², or CR¹³R¹⁴, wherein R¹², R¹³, and R¹⁴ each independently represent an alkyl or alkenyl,
j represents an integer of 1 to 10,
m and n each independently represent an integer of 1 to 50,
and r represents an integer of 0 to 20,
wherein X is a polynucleotide comprising a 5′-cap structure at the 5′-end and of which a hydroxyl group bonded to the 3′-carbon of a nucleoside present at the 3′-end forms a phosphodiester bond together with the terminal of Y.
   [2] A polynucleotide represented by the following formula (I):

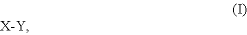

wherein Y is represented by the following formula (II):

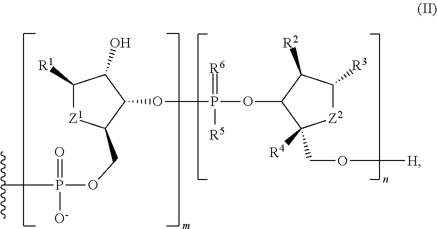

the following formula (III):

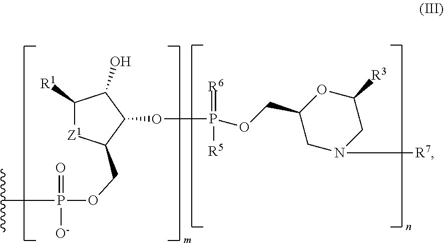

the following formula (IV):

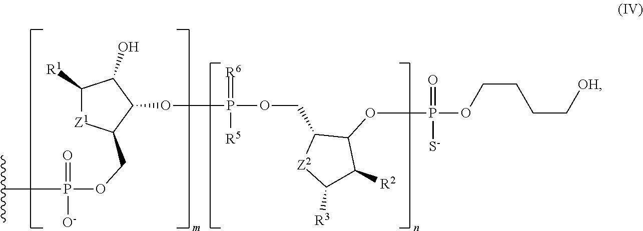

or the following formula (V):

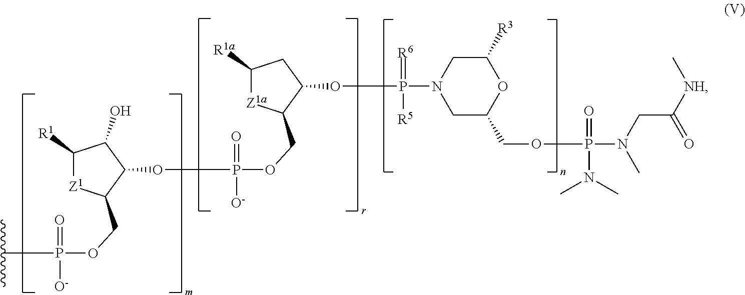

wherein
the wavy line represents a bond to X,
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
   R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II) or formula (IV),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
Z¹, Z^{1a}, and Z² each independently represent O, S, Se, NR¹², or CR¹³R¹⁴, wherein R¹², R¹³, and R¹⁴ each independently represent an alkyl or alkenyl,
m and n each independently represent an integer of 1 to 50,
and r represents an integer of 0 to 20,
wherein X is a polynucleotide comprising a 5′-cap structure at the 5′-end and of which a hydroxyl group bonded to the 3′-carbon of a nucleoside present at the 3′-end forms a phosphodiester bond together with the terminal of Y,
wherein the polynucleotide may be the polynucleotide according to [1].
   [3] The polynucleotide according to [1] or [2], wherein Y is represented by the following formula (II′-A):

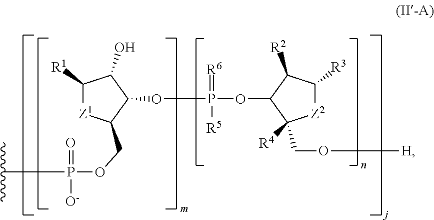

wherein
the wavy line represents a bond to X,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′-A),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
j represents an integer of 1 to 10,
and m and n each independently represent an integer of 1 to 50.
   [4] The polynucleotide according to any one of [1] to [3], wherein Y is represented by the following formula (II′):

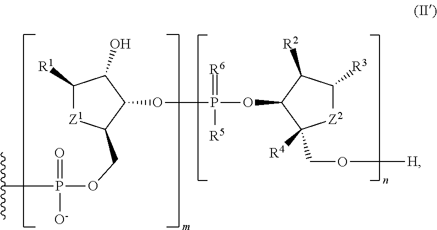

wherein
the wavy line represents a bond to X,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴ wherein R¹², R¹³, and R¹⁴ each independently represent an alkyl or alkenyl,
and m and n each independently represent an integer of 1 to 50.
   [4-1] The polynucleotide according to [1] or [2], wherein Y is represented by the following formula (II′-AX):

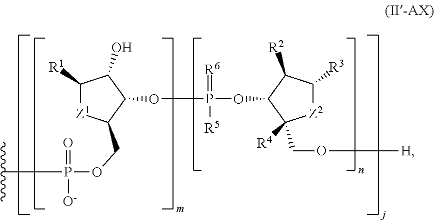

wherein
the wavy line represents a bond to X,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′-AX),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
j represents an integer of 1 to 10,
and m and n each independently represent an integer of 1 to 50.
   [4-2] The polynucleotide according to [1], [2] or [4-1], wherein Y is represented by the following formula (II′-X):

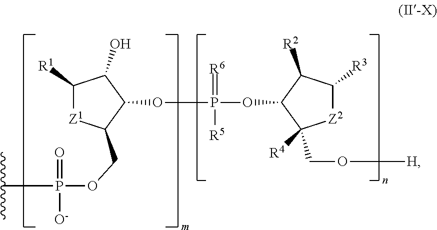

wherein
the wavy line represents a bond to X,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′-X),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
and m and n each independently represent an integer of 1 to 50.
   [5] The polynucleotide according to any one of [1] to [4-2], wherein the nucleobase is thymine, uracil, cytosine, adenine, guanine, 5-methylcytosine, 6-methyladenine, 6-benzyladenine, 1-methyluracil, 5-hydroxymethylcytosine, 2-thiouracil, or hypoxanthine.
   [6] The polynucleotide according to any one of [1] to [5], wherein the nucleobase is thymine, uracil, cytosine, adenine, guanine, or 5-methylcytosine.
   [7] The polynucleotide according to any one of [1] to [6], wherein R¹ is adenine, guanine, cytosine, thymine, or uracil, and R³ is thymine, uracil, cytosine, adenine, guanine, or 5-methylcytosine.
   [8] The polynucleotide according to any one of [1] to [7], wherein R¹ is adenine, cytosine, or uracil, and R³ is thymine, uracil, cytosine, adenine, guanine, or 5-methylcytosine.
   [9] The polynucleotide according to any one of [1] to [8], wherein R¹ is adenine, and R³ is thymine, uracil, cytosine, adenine, guanine, or 5-methylcytosine.
   [10] The polynucleotide according to any one of [1] to [9], wherein R¹ is adenine, and R³ is thymine.
   [11] The polynucleotide according to any one of [1] to [10], wherein R² represents a group obtained by removing one hydrogen atom from a hydroxyl group of a linear or cyclic oligoalkylene glycol having 1 to 40 carbon atoms and 1 to 10 oxygen atoms, a group in which one hydrogen atom on a carbon atom of a cyclic ether having 1 to 40 carbon atoms and 1 to 10 oxygen atoms is replaced by an oxygen atom, a C₁-C₃₀ alkoxy, an optionally substituted benzyloxy, a halogen, a hydroxy, a C₂-C₁₀ alkenyl, or a C₁-C₁₀ alkyl; or together with R⁴, represents a group represented by —O—R¹⁰—, wherein R¹⁰ represents a C₁-C₃ alkylene, and wherein the oxygen atom in the group forms a single bond with the carbon atom to which R² is bonded in formula (II-A), formula (II′-A), formula (II′-AX), formula (II), formula (II′), formula (II′-X), or formula (IV).
   [12] The polynucleotide according to any one of [1] to [11], wherein R² represents a group obtained by removing one hydrogen atom from a hydroxyl group of a linear or cyclic oligoalkylene glycol having 1 to 15 carbon atoms and 1 to 10 oxygen atoms, a group in which one hydrogen atom on a carbon atom of a cyclic ether having 1 to 15 carbon atoms and 1 to 10 oxygen atoms is replaced by an oxygen atom, a C₁-C₂₀ alkoxy, a benzyloxy, fluoro, a C₂-C₁₀ alkenyl, or a C₁-C₁₀ alkyl, and R⁴ represents H.
   [13] The polynucleotide according to any one of [1] to [12], wherein R² represents a group represented by CH₃—(O—CH₂—CH₂—)ₚO— or (CH₃O(—CH₂—CH₂—O—)_{w}CH₂—)₂CH—O—, in which p represents an integer of 1 to 2 and w represents an integer of 0 to 2; a group in which one hydrogen atom on a carbon atom of tetrahydrofuran or tetrahydropyran is replaced by an oxygen atom; a C₁-C₁₆ alkoxy; a benzyloxy; a C₂-C₅ alkenyl; or a C₁-C₅ alkyl, and R⁴ represents H.
   [14] The polynucleotide according to any one of [1] to [13], wherein R² is selected from the group consisting of methoxy-ethyleneoxy, methoxy, a benzyloxy, allyl, and a group represented by the following formulas:

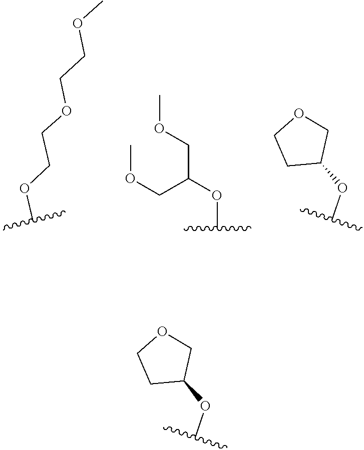

and R⁴ represents H.
[15] The polynucleotide according to any one of [1] to [14], wherein R² represents methoxy-ethyleneoxy, and R⁴ represents H.
[16] The polynucleotide according to any one of [1] to [15], wherein R⁵ represents O.
[17] The polynucleotide according to any one of [1] to [16], wherein R⁶ represents O.
[18] The polynucleotide according to any one of [1] to [17], wherein R⁵ is O⁻ or S⁻, and R⁶ is O.
[19] The polynucleotide according to any one of [1] to [18], wherein R⁵ represents O, and R⁶ represents O.
[20] The polynucleotide according to any one of [1] to [19], wherein Z¹ and Z² represent O.
[21] The polynucleotide according to any one of [1] to [20], wherein n represents an integer of 1 to 20, 1 to 15, 1 to 10, 1 to 7, 1 to 5, 1 to 4, 1 to 3, or 1 to 2.
[22] The polynucleotide according to any one of [1] to [21], wherein n represents 1 or 2.
[23] The polynucleotide according to any one of [1] to [22], wherein n represents 1.
[24] The polynucleotide according to any one of [1] to [23], wherein j represents 1 or 2.
[25] The polynucleotide according to any one of [1] to [24], wherein j represents 1.
[26] The polynucleotide according to any one of [1] to [25], wherein Y is represented by the following formula (VI):

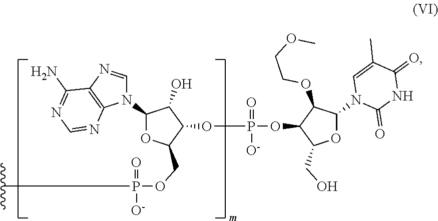

wherein the wavy line represents a bond to X, and m represents an integer of 1 to 20.
[27] The polynucleotide according to any one of [1] to [26], wherein X has a polyadenyl region at the 3′-end.
[28] The polynucleotide according to any one of [1] to [27], wherein X is a polyribonucleotide comprising a 5′-cap structure at the 5′-end and of which a hydroxyl group bonded to the 3′-carbon of a nucleoside present at the 3′-end forms a phosphodiester bond together with the terminal of Y.
[29] The polynucleotide according to any one of [1] to [28], wherein X is a polyribonucleotide comprising a 5′-cap structure at the 5′-end and a polyadenyl region at the 3′-end, and of which a hydroxyl group bonded to the 3′-carbon of a nucleoside present at the 3′-end forms a phosphodiester bond together with the terminal of Y.
[30] A composition comprising the polynucleotide according to any one of [1] to [29].
[31] A producing method of the polynucleotide according to any one of [1] to [29].
[32] A producing method of the polynucleotide according to any one of [1] to [29], comprising contacting a polynucleotide represented by the following formula (VII):

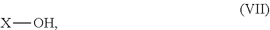

wherein X is the same as X in formula (I), and OH represents a hydroxyl group bonded to the 3′-carbon of a nucleoside present at the 3′-end of X, with an oligonucleotide represented by the following formula (VIII-A):

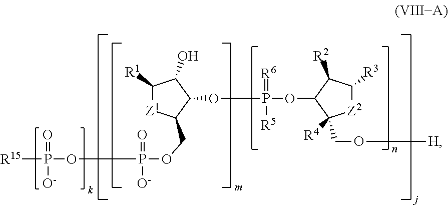

the following formula (IX):

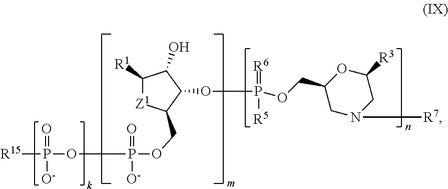

the following formula (X):

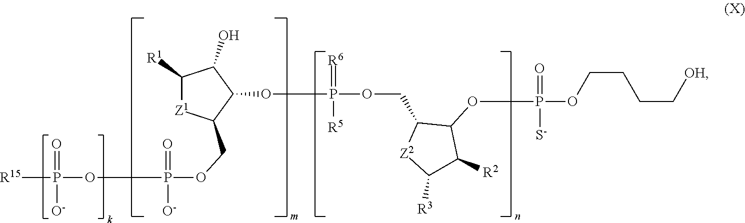

or the following formula (XI):

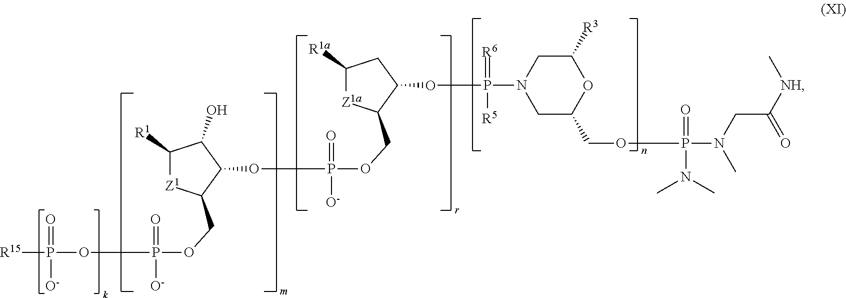

wherein
R¹ to R⁷, R^{1a}, Z¹, Z^{1a}, Z², j, m, n, and r are the same as those in formula (II-A), formula (III), formula (VI), or formula (V),
R¹⁵ represents O⁻ or a group obtained by removing one hydrogen atom from a hydroxyl group of the hydroxymethyl group at the 5′-position of adenosine,
and k represents an integer of 0 to 2, in the presence of RNA ligase or poly(A) polymerase to obtain the polynucleotide represented by formula (I)
wherein the producing method may be the producing method according to [31].
   [33] A producing method of the polynucleotide according to any one of [2] to [29], comprising contacting a polynucleotide represented by the following formula (VII):

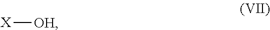

wherein X is the same as X in formula (I), and OH represents a hydroxyl group bonded to the 3′-carbon of a nucleoside present at the 3′-end of X, with an oligonucleotide represented by the following formula (VIII):

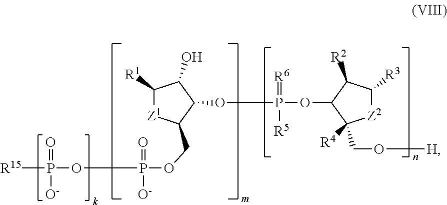

the following formula (IX):

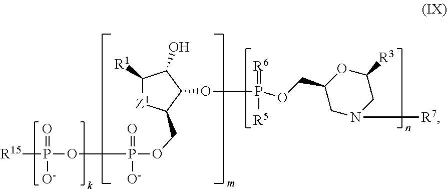

the following formula (X):

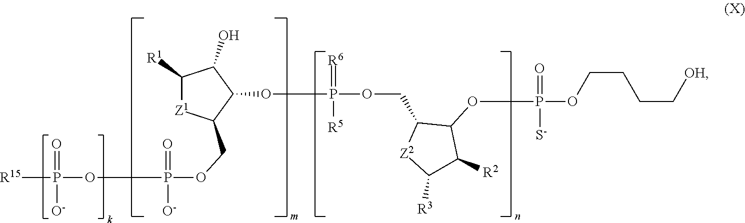

or the following formula (XI):

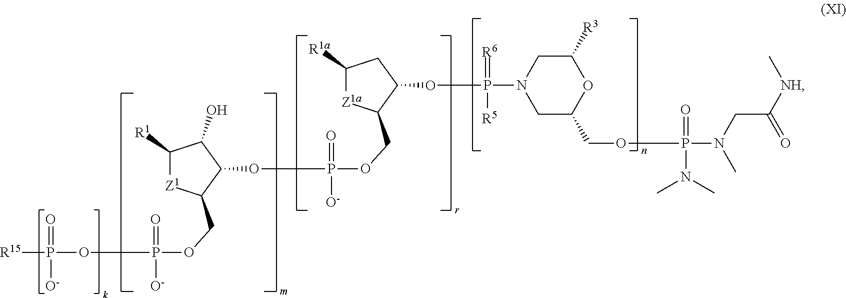

wherein
R¹ to R⁷, R^{1a}, Z¹, Z^{1a}, Z², m, n, and r are the same as those in formula (II), formula (III), formula (VI), or formula (V),
R¹⁵ represents O⁻ or a group obtained by removing one hydrogen atom from a hydroxyl group of the hydroxymethyl group at the 5′-position of adenosine,
and k represents an integer of 0 to 2, in the presence of RNA ligase or poly(A) polymerase to obtain the polynucleotide represented by formula (I),
wherein the producing method may be the producing method according to or [32].
   [34] A producing method of the polynucleotide according to any one of [3] to [4] and [5] to [29], comprising contacting a polynucleotide represented by the following formula (VII):

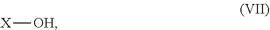

wherein X is the same as X in formula (I), and OH represents a hydroxyl group bonded to the 3′-carbon of a nucleoside present at the 3′-end of X, with an oligonucleotide represented by the following formula (VIII′-A):

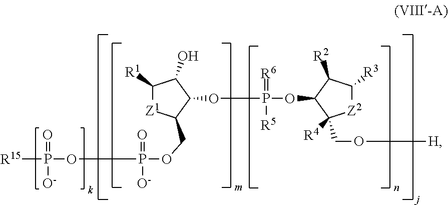

wherein
R¹ to R⁶, Z¹, Z, j, m, and n are the same as those in formula (II′-A),
R¹⁵ represents O⁻ or a group obtained by removing one hydrogen atom from a hydroxyl group of the hydroxymethyl group at the 5′-position of adenosine,
and k represents an integer of 0 to 2,
   in the presence of RNA ligase or poly(A) polymerase to obtain the polynucleotide represented by formula (I),
wherein the producing method may be the producing method according to any one of [31] to [33].
   [35] A producing method of the polynucleotide according to any one of [4] and [5] to [29], comprising contacting a polynucleotide represented by the following formula (VII):

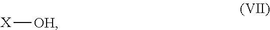

wherein X is the same as X in formula (I), and OH represents a hydroxyl group bonded to the 3′-carbon of a nucleoside present at the 3′-end of X, with an oligonucleotide represented by the following formula (VIII′):

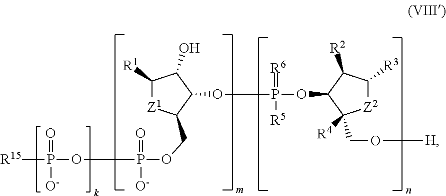

wherein
R¹ to R⁶, Z¹, Z², m, and n are the same as those in formula (II′),
R¹⁵ represents O⁻ or a group obtained by removing one hydrogen atom from a hydroxyl group of the hydroxymethyl group at the 5′-position of adenosine,
and k represents an integer of 0 to 2,
   in the presence of RNA ligase or poly(A) polymerase to obtain the polynucleotide represented by formula (I),
wherein the producing method may be the producing method according to any one of [31] to [34].
   [35-1] A producing method of the polynucleotide according to any one of [4-1] to [29], comprising contacting a polynucleotide represented by the following formula (VII):

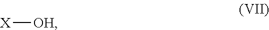

wherein X is the same as X in formula (I), and OH represents a hydroxyl group bonded to the 3′-carbon of a nucleoside present at the 3′-end of X,
with an oligonucleotide represented by the following formula (VIII′-AX):

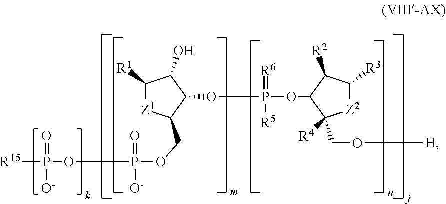

wherein
R¹ to R⁶, Z¹, Z², j, m, and n are the same as those in formula (II′-AX),
R¹⁵ represents O⁻ or a group obtained by removing one hydrogen atom from a hydroxyl group of the hydroxymethyl group at the 5′-position of adenosine,
and k represents an integer of 0 to 2,
   in the presence of RNA ligase or poly(A) polymerase to obtain the polynucleotide represented by formula (I),
wherein the producing method may be the producing method according to any one of [31] to [33].
   [35-2] A producing method of the polynucleotide according to any one of [4-2] to [29], comprising contacting a polynucleotide represented by the following formula (VII):

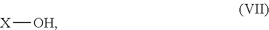

wherein X is the same as X in formula (I), and OH represents a hydroxyl group bonded to the 3′-carbon of a nucleoside present at the 3′-end of X,
with an oligonucleotide represented by the following formula (VIII′-X):

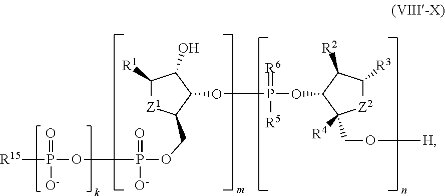

wherein
R¹ to R⁶, Z¹, Z², m, and n are the same as those in formula (II′-X),
R¹⁵ represents O⁻ or a group obtained by removing one hydrogen atom from a hydroxyl group of the hydroxymethyl group at the 5′-position of adenosine,
and k represents an integer of 0 to 2,
   in the presence of RNA ligase or poly(A) polymerase to obtain the polynucleotide represented by formula (I), wherein the producing method may be the producing method according to any one of [31] to and [35-1].
   [36] A producing method of the polynucleotide according to any one of [22] to [29], comprising contacting a polynucleotide represented by the following formula (VII):

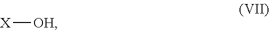

wherein X is the same as X in formula (I), and OH represents a hydroxyl group bonded to the 3′-carbon of a nucleoside present at the 3′-end of X,
with an oligonucleotide represented by the following formula (XII):

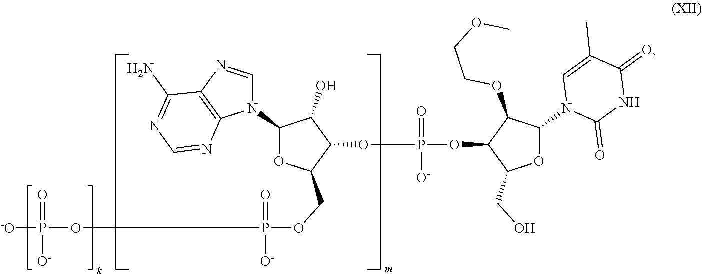

wherein
m is the same as those in formula (VI),
and k represents an integer of 0 to 2,
   in the presence of RNA ligase or poly(A) polymerase to obtain the polynucleotide represented by formula (I),
wherein the producing method may be the producing method according to any one of [31] to [35].
   [37] The producing method according to any one of [31] to [36], wherein m represents an integer of 1 to 20, 1 to 15, 1 to 12, 1 to 10, 1 to 9, 1 to 8, 1 to 7, 1 to 6, 1 to 5, 1 to 4, 1 to 3, or 1 to 2.
   [38] The producing method according to any one of [31] to [37], wherein the RNA ligase or poly(A) polymerase is RNA ligase.
   [39] An oligonucleotide represented by the following formula (VIII-A):

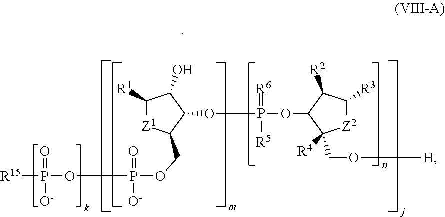

the following formula (IX):

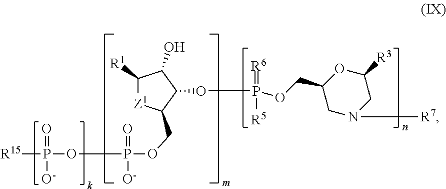

the following formula (X):

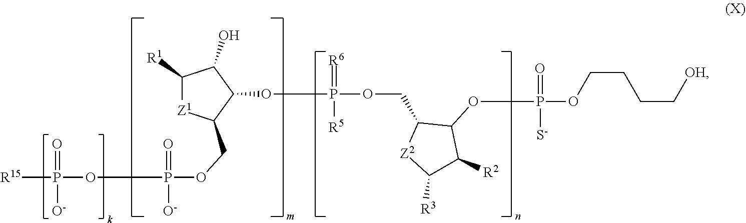

or the following formula (XI):

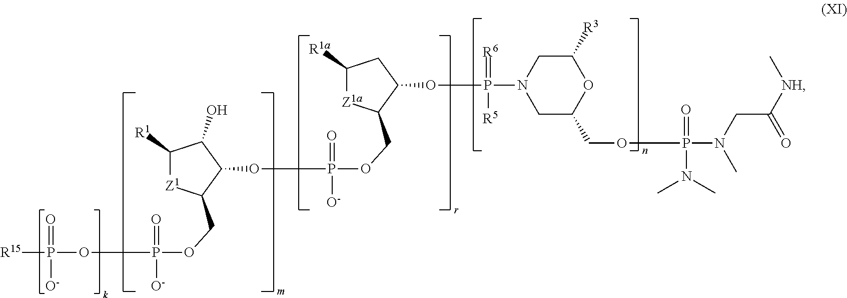

wherein
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (VIII-A) or formula (X),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
R¹⁵ represents O⁻ or a group obtained by removing one hydrogen atom from a hydroxyl group of the hydroxymethyl group at the 5′-position of adenosine,
Z¹, Z^{1a}, and Z² each independently represent O, S, Se, NR¹², or CR¹³R¹⁴, wherein R¹², R¹³, and R¹⁴ each independently represent an alkyl or alkenyl,
j represents an integer of 1 to 10,
k represents an integer of 0 to 2,
m and n each independently represent an integer of 1 to 50,
and r represents an integer of 0 to 20.
   [40] An oligonucleotide represented by the following formula (VIII):

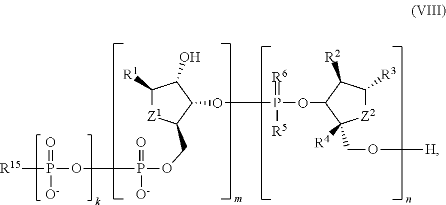

the following formula (IX):

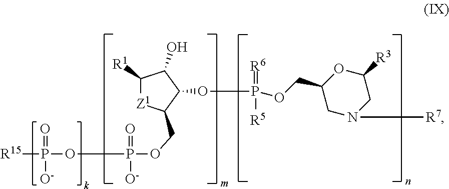

the following formula (X):

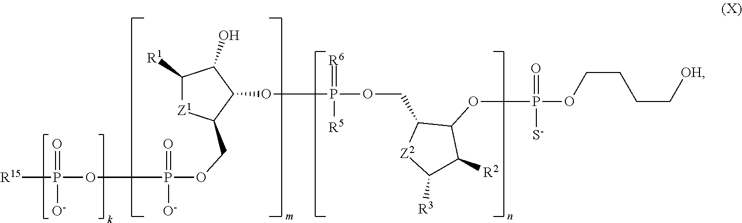

or the following formula (XI):

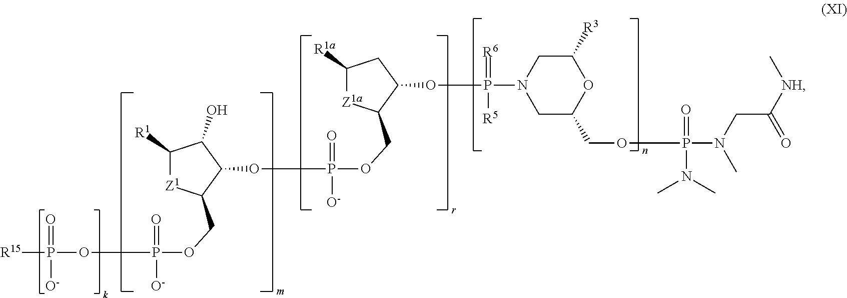

wherein
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (VIII) or formula (X),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
R¹⁵ represents O⁻ or a group obtained by removing one hydrogen atom from a hydroxyl group of the hydroxymethyl group at the 5′-position of adenosine,
Z¹, Z^{1a}, and Z² each independently represent O, S, Se, NR¹², or CR¹³R¹⁴, wherein R¹², R¹³, and R¹⁴ each independently represent an alkyl or alkenyl,
k represents an integer of 0 to 2,
m and n each independently represent an integer of 1 to 50,
and r represents an integer of 0 to 20,
wherein the oligonucleotide may be the oligonucleotide according to [39].
   [41] An oligonucleotide represented by the following formula (VIII′-A):

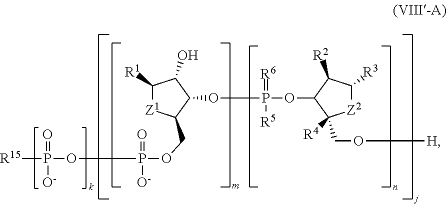

wherein
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (VIII′-A),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R¹⁵ represents O⁻ or a group obtained by removing one hydrogen atom from a hydroxyl group of the hydroxymethyl group at the 5′-position of adenosine,
Z¹ and Z² each independently represent O, S, Se, NR¹², or CR¹³R¹⁴ wherein R¹², R¹³, and R¹⁴ each independently represent an alkyl or alkenyl,
j represents an integer of 1 to 10,
k represents an integer of 0 to 2,
and m and n each independently represent an integer of 1 to 50,
wherein the oligonucleotide may be the oligonucleotide according to [39] or [40].
   [42] An oligonucleotide represented by the following formula (VIII′):

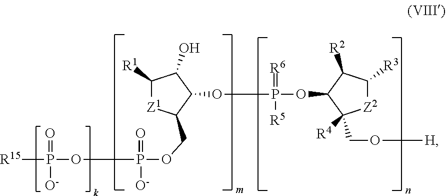

wherein
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (VIII′),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R¹⁵ represents O⁻ or a group obtained by removing one hydrogen atom from a hydroxyl group of the hydroxymethyl group at the 5′-position of adenosine,
Z¹ and Z² each independently represent O, S, Se, NR¹², or CR¹³R¹⁴, wherein R¹², R¹³, and R¹⁴ each independently represent an alkyl or alkenyl,
k represents an integer of 0 to 2,
and m and n each independently represent an integer of 1 to 50,
wherein the oligonucleotide may be the oligonucleotide according to any one of [39] to [41].
   [42-1] An oligonucleotide represented by the following formula (VIII′-AX):

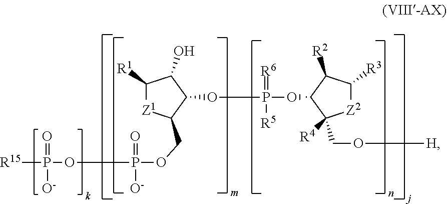

wherein
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (VIII′-AX),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R¹⁵ represents O⁻ or a group obtained by removing one hydrogen atom from a hydroxyl group of the hydroxymethyl group at the 5′-position of adenosine,
Z¹ and Z² each independently represent O, S, Se, NR¹², or CR¹³R¹⁴, wherein R¹², R¹³, and R¹⁴ each independently represent an alkyl or alkenyl,
j represents an integer of 1 to 10,
k represents an integer of 0 to 2,
and m and n each independently represent an integer of 1 to 50,
wherein the oligonucleotide may be the oligonucleotide according to [39] or [40].
   [42-2] An oligonucleotide represented by the following formula (VIII′-X):

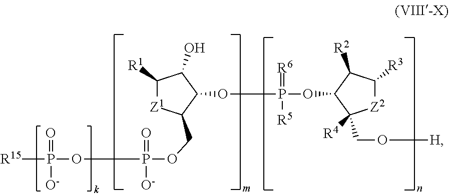

wherein
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (VIII′-X),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R¹⁵ represents O⁻ or a group obtained by removing one hydrogen atom from a hydroxyl group of the hydroxymethyl group at the 5′-position of adenosine,
Z¹ and Z² each independently represent O, S, Se, NR¹², or CR¹³R¹⁴ wherein R¹², R¹³, and R¹⁴ each independently represent an alkyl or alkenyl,
k represents an integer of 0 to 2,
and m and n each independently represent an integer of 1 to 50,
wherein the oligonucleotide may be the oligonucleotide according to [39], [40] or [42-1].
   [43] The oligonucleotide according to any one of [39] to [42-2], wherein the nucleobase is thymine, uracil, cytosine, adenine, guanine, 5-methylcytosine, 6-methyladenine, 6-benzyladenine, 1-methyluracil, 5-hydroxymethylcytosine, 2-thiouracil, or hypoxanthine.
   [44] The oligonucleotide according to any one of [39] to [43], wherein the nucleobase is thymine, uracil, cytosine, adenine, guanine, or 5-methylcytosine.
   [45] The oligonucleotide according to any one of [39] to [44], wherein R¹ is adenine, guanine, cytosine, thymine, or uracil, and R³ is thymine, uracil, cytosine, adenine, guanine, or 5-methylcytosine.
   [46] The oligonucleotide according to any one of [39] to [45], wherein R¹ is adenine, cytosine, or uracil, and R³ is thymine, uracil, cytosine, adenine, guanine, or 5-methylcytosine.
   [47] The oligonucleotide according to any one of [39] to [46], wherein R¹ is adenine, and R³ is thymine, uracil, cytosine, adenine, guanine, or 5-methylcytosine.
   [48] The oligonucleotide according to any one of [39] to [47], wherein R¹ is adenine, and R³ is thymine.
   [49] The oligonucleotide according to any one of [39] to [48], wherein R² represents a group obtained by removing one hydrogen atom from a hydroxyl group of a linear or cyclic oligoalkylene glycol having 1 to 40 carbon atoms and 1 to 10 oxygen atoms, a group in which one hydrogen atom on a carbon atom of a cyclic ether having 1 to 40 carbon atoms and 1 to 10 oxygen atoms is replaced by an oxygen atom, a C₁-C₃₀ alkoxy, an optionally substituted benzyloxy, a halogen, a hydroxy, a C₂-C₁₀ alkenyl, or a C₁-C₁₀ alkyl; or together with R⁴, represents a group represented by —O—R¹⁰—, wherein R¹⁰ represents a C₁-C₃ alkylene, and wherein the oxygen atom in the group forms a single bond with the carbon atom to which R² is bonded in formula (VIII-A), formula (VIII′-A), formula (VIII′-AX), formula (VIII), formula (VIII′), formula (VIII′-X), or formula (X).
   [50] The oligonucleotide according to any one of [39] to [49], wherein R² represents a group obtained by removing one hydrogen atom from a hydroxyl group of a linear or cyclic oligoalkylene glycol having 1 to 15 carbon atoms and 1 to 10 oxygen atoms, a group in which one hydrogen atom on a carbon atom of a cyclic ether having 1 to 15 carbon atoms and 1 to 10 oxygen atoms is replaced by an oxygen atom, a C₁-C₂₀ alkoxy, a benzyloxy, fluoro, a C₂-C₁₀ alkenyl, or a C₁-C₁₀ alkyl, and R⁴ represents H.
   [51] The oligonucleotide according to any one of [39] to [50], wherein R² represents a group represented by CH₃—(O—CH₂—CH₂—)ₚO— or (CH₃O(—CH₂—CH₂—O—)_{w}CH₂—)₂CH—O—, in which p represents an integer of 1 to 2 and w represents an integer of 0 to 2; a group in which one hydrogen atom on a carbon atom of tetrahydrofuran or tetrahydropyran is replaced by an oxygen atom; a C₁-C₁₆ alkoxy; a benzyloxy; a C₂-C₅ alkenyl; or a C₁-C₅ alkyl, and R⁴ represents H.
   [52] The oligonucleotide according to any one of [39] to [51], wherein R² is selected from the group consisting of methoxy-ethyleneoxy, methoxy, a benzyloxy, allyl, and a group represented by the following formulas:

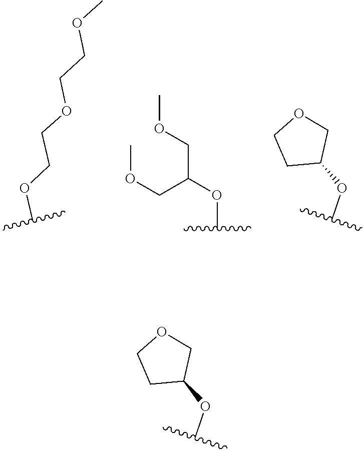

and R⁴ represents H.
[53] The oligonucleotide according to any one of [39] to [52], wherein R² represents methoxy-ethyleneoxy, and R⁴ represents H.
[54] The oligonucleotide according to any one of [39] to [53], wherein R⁵ represents O⁻.
[55] The oligonucleotide according to any one of [39] to [54], wherein R⁶ represents O.
[56] The oligonucleotide according to any one of [39] to [55], wherein R⁵ is O⁻ or S⁻, and R⁶ is O.
[57] The oligonucleotide according to any one of [39] to [56], wherein R⁵ represents O⁻, and R⁶ represents O.
[58] The oligonucleotide according to any one of [39] to [57], wherein Z¹ and Z² represent O.
[59] The oligonucleotide according to any one of [39] to [58], wherein n represents an integer of 1 to 20, 1 to 15, 1 to 10, 1 to 7, 1 to 5, 1 to 4, 1 to 3, or 1 to 2.
[60] The oligonucleotide according to any one of [39] to [59], wherein n represents 1 or 2.
[61] The oligonucleotide according to any one of [39] to [60], wherein n represents 1.
[62] The oligonucleotide according to any one of [39] to [61], wherein j represents 1 or 2.
[63] The oligonucleotide according to any one of [39] to [62], wherein j represents 1.
[64] An oligonucleotide represented by the following formula (XII):

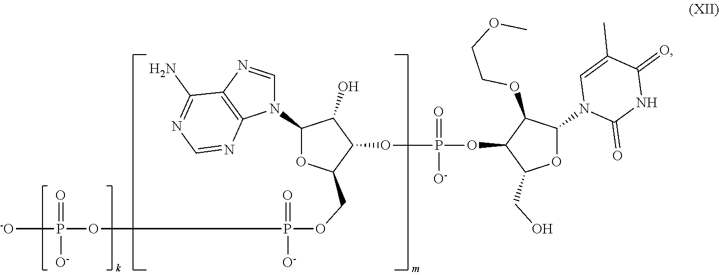

wherein
m represents an integer of 1 to 20,
and k represents an integer of 0 to 2,
wherein the oligonucleotide may be the oligonucleotide according to any one of [39] to [63].
   [65] The oligonucleotide according to any one of [39] to [64], wherein m represents an integer of 1 to 20, 1 to 15, 1 to 12, 1 to 10, 1 to 9, 1 to 8, 1 to 7, 1 to 6, 1 to 5, 1 to 4, 1 to 3, or 1 to 2.
   [66] The oligonucleotide according to any one of [39] to [65], for use in the improvement of an in vivo stability of a polynucleotide.
   [67] Use or application of the oligonucleotide according to any one of [39] to [65] in improving an in vivo stability of a polynucleotide.
   [68] A kit comprising the oligonucleotide according to any one of [39] to [65], for improving an in vivo stability of a polynucleotide.
   [69] A method of improving the stability of a polynucleotide in vivo, comprising introducing a group represented by the following formula (II-A):

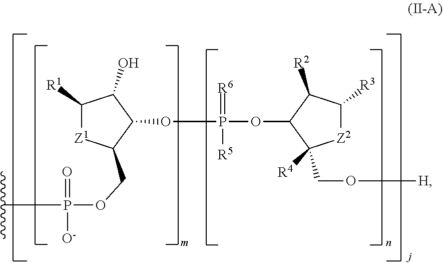

the following formula (III):

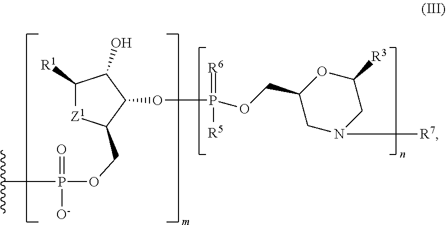

the following formula (IV):

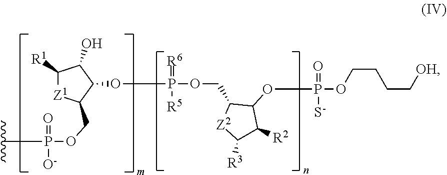

or the following formula (V):

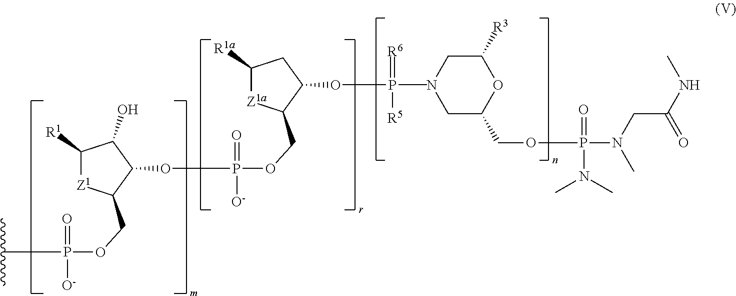

to the 3′-end of the polynucleotide,
wherein
the wavy line represents a bond to the polynucleotide,
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II-A) or formula (IV),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
Z¹, Z^{1a}, and Z² each independently represent O, S, Se, NR¹², or CR¹³R¹⁴, wherein R¹², R¹³, and R¹⁴ each independently represent an alkyl or alkenyl,
j represents an integer of 1 to 10,
m and n each independently represent an integer of 1 to 50,
and r represents an integer of 0 to 20.
   [70] A method of improving the stability of a polynucleotide in vivo, comprising introducing a group represented by the following formula (II):

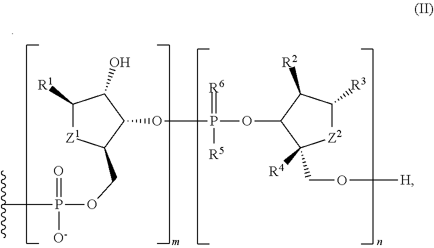

the following formula (III):

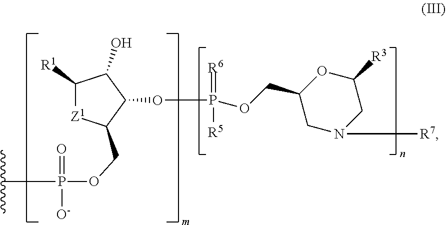

the following formula (IV):

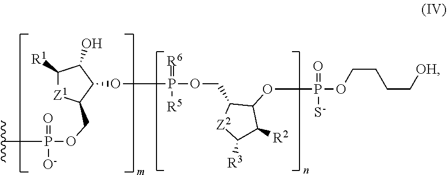

the following formula (V):

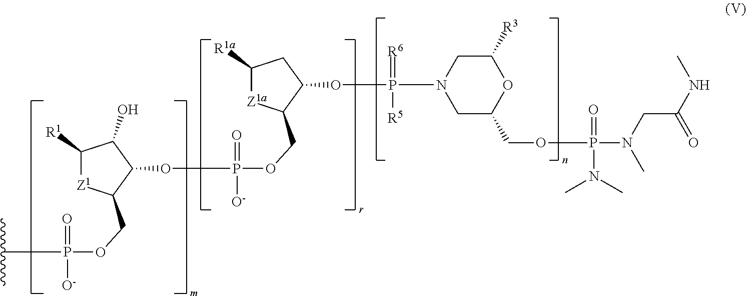

to the 3′-end of the polynucleotide,
wherein
the wavy line represents a bond to the polynucleotide,
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II) or formula (IV),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
Z¹, Z^{1a}, and Z² each independently represent O, S, Se, NR¹², or CR¹³R¹⁴, wherein R¹², R¹³, and R¹⁴ each independently represent an alkyl or alkenyl,
m and n each independently represent an integer of 1 to 50,
and r represents an integer of 0 to 20,
wherein the method may be the method according to [69].
   [71] A method of improving an expression level of a protein encoded by a coding region in a polyribonucleotide, comprising introducing a group represented by the following formula (II-A):

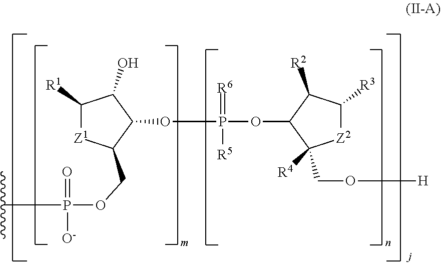

the following formula (III):

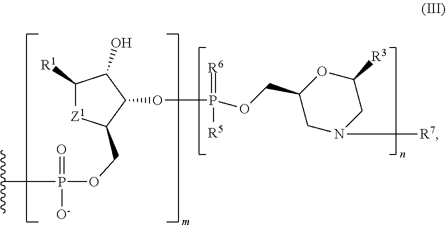

the following formula (IV):

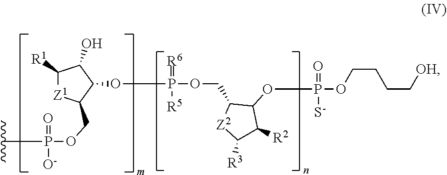

or the following formula (V):

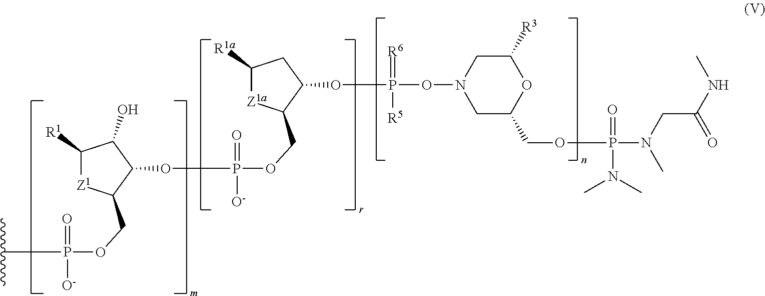

to the 3′-end of the polyribonucleotide,
wherein
the wavy line represents a bond to the polyribonucleotide,
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II-A) or formula (IV),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
Z¹, Z^{1a}, and Z² each independently represent O, S, Se, NR¹², or CR¹³R¹⁴, wherein R¹², R¹³, and R¹⁴ each independently represent an alkyl or alkenyl,
j represents an integer of 1 to 10,
m and n each independently represent an integer of 1 to 50,
and r represents an integer of 0 to 20.
   [72] A method of improving an expression level of a protein encoded by a coding region in a polyribonucleotide, comprising introducing a group represented by the following formula (II):

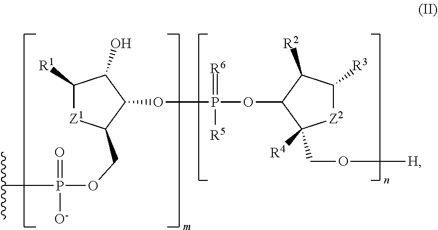

the following formula (III):

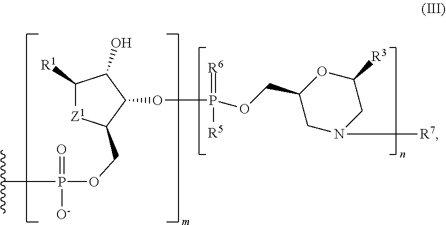

the following formula (IV):

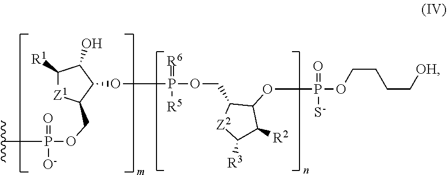

the following formula (V):

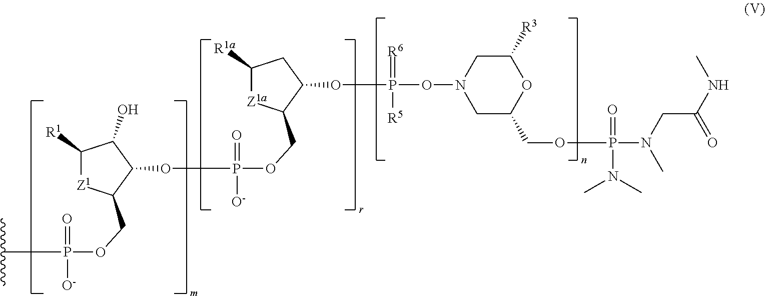

to the 3′-end of the polyribonucleotide,
wherein
the wavy line represents a bond to the polyribonucleotide,
R¹, R^{1a}, and R³ each independently represent a nucleobase, wherein if Y has two or more R¹, then each of the two or more R¹ may independently represent a nucleobase; wherein if Y has two or more R^{1a}, then each of the two or more R^{1a} may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II) or formula (IV),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
R⁷ represents H or a group represented by R¹¹—C(═O)—, wherein R¹¹ represents an alkyl,
Z¹, Z^{1a}, and Z² each independently represent O, S, Se, NR¹², or CR¹³R¹⁴, wherein R¹², R¹³, and R¹⁴ each independently represent an alkyl or alkenyl,
m and n each independently represent an integer of 1 to 50,
and r represents an integer of 0 to 20,
wherein the method may be the method according to [71].
   [73] The method according to any one of [69] or [71], wherein the group represented by the formula (II-A), the formula (III), the formula (IV), or the formula (V) is represented by the following formula (II′-A):

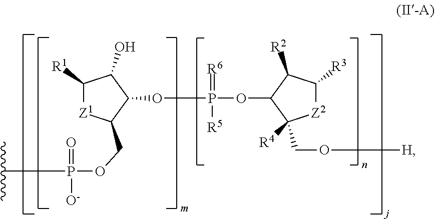

wherein
the wavy line represents a bond to the polynucleotide or the polyribonucleotide,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′-A),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
j represents an integer of 1 to 10,
and m and n each independently represent an integer of 1 to 50.
   [74] The method according to any one of [70] or [72], wherein the group represented by formula (II), formula (III), formula (IV), or formula (V) is represented by the following formula (II′):

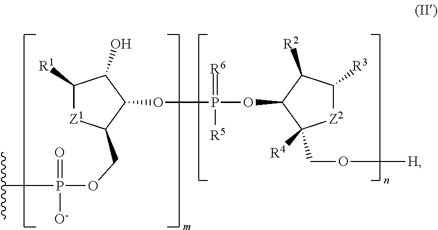

wherein
the wavy line represents a bond to the polynucleotide or the polyribonucleotide,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
and m and n each independently represent an integer of 1 to 50.
   [74-1] The method according to any one of [69] or [71], wherein the group represented by the formula (II-A), the formula (III), the formula (IV), or the formula (V) is represented by the following formula (IT′-AX):

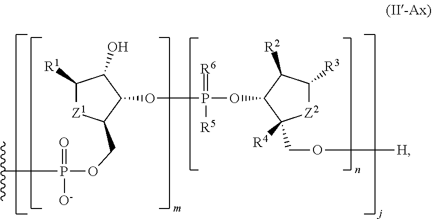

wherein
the wavy line represents a bond to the polynucleotide or the polyribonucleotide,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′-AX),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
j represents an integer of 1 to 10,
and m and n each independently represent an integer of 1 to 50.
   [74-2] The method according to any one of [70] or [72], wherein the group represented by formula (II), formula (III), formula (IV), or formula (V) is represented by the following formula (II′-X):

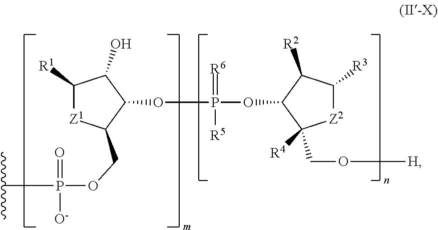

wherein
the wavy line represents a bond to the polynucleotide or the polyribonucleotide,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II′-X),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
and m and n each independently represent an integer of 1 to 50.
   [75] The method according to any one of [69] to [74-2], wherein the nucleobase is thymine, uracil, cytosine, adenine, guanine, 5-methylcytosine, 6-methyladenine, 6-benzyladenine, 1-methyluracil, 5-hydroxymethylcytosine, 2-thiouracil, or hypoxanthine.
   [76] The method according to any one of [69] to [75], wherein the nucleobase is thymine, uracil, cytosine, adenine, guanine, or 5-methylcytosine.
   [77] The method according to any one of [69] to [76], wherein R¹ is adenine, guanine, cytosine, thymine, or uracil, and R³ is thymine, uracil, cytosine, adenine, guanine, or 5-methylcytosine.
   [78] The method according to any one of [69] to [77], wherein R¹ is adenine, cytosine, or uracil, and R³ is thymine, uracil, cytosine, adenine, guanine, or 5-methylcytosine.
   [79] The method according to any one of [69] to [78], wherein R¹ is adenine, and R³ is thymine, uracil, cytosine, adenine, guanine, or 5-methylcytosine.
   [80] The method according to any one of [69] to [79], wherein R¹ is adenine, and R³ is thymine.
   [81] The method according to any one of [69] to [80], wherein R² represents a group obtained by removing one hydrogen atom from a hydroxyl group of a linear or cyclic oligoalkylene glycol having 1 to 40 carbon atoms and 1 to 10 oxygen atoms, a group in which one hydrogen atom on a carbon atom of a cyclic ether having 1 to 40 carbon atoms and 1 to 10 oxygen atoms is replaced by an oxygen atom, a C₁-C₃₀ alkoxy, an optionally substituted benzyloxy, a halogen, a hydroxy, a C₂-C₁₀ alkenyl, or a C₁-C₁₀ alkyl; or together with R⁴, represents a group represented by —O—R¹⁰—, wherein R¹⁰ represents a C₁-C₃ alkylene, and wherein the oxygen atom in the group forms a single bond with the carbon atom to which R² is bonded in formula (II-A), formula (II′-A), formula (II′-AX), formula (II), formula (II′), formula (II′-X), or formula (IV).
   [82] The method according to any one of [69] to [81], wherein R² represents a group obtained by removing one hydrogen atom from a hydroxyl group of a linear or cyclic oligoalkylene glycol having 1 to 15 carbon atoms and 1 to 10 oxygen atoms, a group in which one hydrogen atom on a carbon atom of a cyclic ether having 1 to 15 carbon atoms and 1 to 10 oxygen atoms is replaced by an oxygen atom, a C₁-C₂₀ alkoxy, a benzyloxy, fluoro, a C₂-C₁₀ alkenyl, or a C₁-C₁₀ alkyl, and R⁴ represents H.
   [83] The method according to any one of [69] to [82], wherein R² represents a group represented by CH₃—(O—CH₂—CH₂—)ₚO— or (CH₃O(—CH₂—CH₂—O—)_{w}CH₂—)₂CH—O—, in which p represents an integer of 1 to 2 and w represents an integer of 0 to 2; a group in which one hydrogen atom on a carbon atom of tetrahydrofuran or tetrahydropyran is replaced by an oxygen atom; a C₁-C₁₆ alkoxy; a benzyloxy; a C₂-C₅ alkenyl; or a C₁-C₅ alkyl, and R⁴ represents H.
   [84] The method according to any one of [69] to [83], wherein R² is selected from the group consisting of methoxy-ethyleneoxy, methoxy, a benzyloxy, allyl, and a group represented by the following formulas:

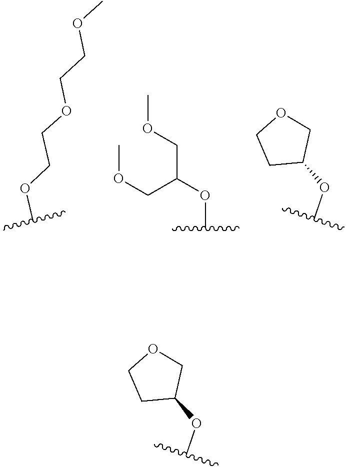

and R⁴ represents H.
[85] The method according to any one of [69] to [84], wherein R² represents methoxy-ethyleneoxy, and R⁴ represents H.
[86] The method according to any one of [69] to [85], wherein R⁵ represents O⁻.
[87] The method according to any one of [69] to [86], wherein R⁶ represents O.
[88] The method according to any one of [69] to [87], wherein R⁵ is O⁻ or S⁻, and R⁶ is O.
[89] The method according to any one of [69] to [88], wherein R⁵ represents O, and R⁶ represents O.
[90] The method according to any one of [69] to [89], wherein Z¹ and Z² represent O.
[91] The method according to any one of [69] to [90], wherein n represents an integer of 1 to 20, 1 to 15, 1 to 10, 1 to 7, 1 to 5, 1 to 4, 1 to 3, or 1 to 2.
[92] The method according to any one of [69] to [91], wherein n represents 1 or 2.
[93] The method according to any one of [69] to [92], wherein n represents 1.
[94] The method according to any one of [69] to [93], wherein j represents 1 or 2.
[95] The method according to any one of [69] to [94], wherein j represents 1.
[96] The method according to any one of [69] or [95], wherein the group represented by formula (II), formula (III), formula (IV), or formula (V) is represented by the following formula (VI):

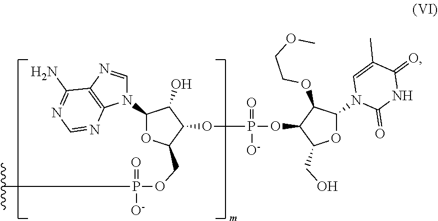

wherein the wavy line represents a bond to the polynucleotide or the polyribonucleotide, and m represents an integer of 1 to 20.

According to the present disclosure, a polynucleotide with improved stability in vivo (in a body) is provided. According to the present disclosure, a producing method of a polynucleotide with improved stability in vivo is provided. According to the present disclosure, a method of improving the stability of a polynucleotide in vivo is provided.

The polynucleotide according to one embodiment of the present disclosure has high purity. According to the producing method of the polynucleotide according to one embodiment of the present disclosure, the polynucleotide can be produced with high purity.

In a case where the polynucleotide according to one embodiment of the present disclosure is a polyribonucleotide, the expression level of the protein encoded by the coding region in the polyribonucleotide is increased in a subject to whom the polynucleotide according to the present disclosure is administered.

The polynucleotide according to one embodiment of the present disclosure has improved stability in vivo even if the nucleotide length of the oligonucleotide or its derivative with a specific structure introduced to the 3′-end is extremely short. As a result, for example, since it is acceptable for the nucleotide length of the building blocks used for the introduction to be short, liquid-phase synthesis of the building blocks becomes possible, thereby enabling industrial production at low cost and high efficiency.

### BRIEF DESCRIPTION OF THE DRAWINGS

FIG. 1 shows the results of HPLC analysis of EPO mRNA and PolyA-modified EPO-mRNA in Example 1.

### DETAILED DESCRIPTION

Embodiments for carrying out the present disclosure are described below, but the present disclosure is not limited to the following explanations.

As used herein, the term “halogen atom” and “halogen” mean a fluorine atom, chlorine atom, bromine atom, or iodine atom.

As used herein, the term “alkyl” means a monovalent group derived from an alkane by removing one hydrogen atom. The term “Cₘ-Cₙ alkyl” (where m and n are positive integers) as used herein means an alkyl group having m to n carbon atoms.

As used herein, the term “C₁-C₆ alkyl” means a monovalent group derived from an aliphatic saturated hydrocarbon having 1 to 6 carbon atoms by removing any one hydrogen atom, and refers to a straight-chain or branched-chain alkyl having 1 to 6 carbon atoms or a cyclic alkyl having 3 to 6 carbon atoms. In some embodiments, C₁-C₆ alkyl comprise methyl, ethyl, n-propyl, isopropyl, n-butyl, isobutyl, sec-butyl, tert-butyl, n-pentyl, isopentyl, sec-pentyl, neopentyl, 1-methylbutyl, 2-methylbutyl, 1,1-dimethylpropyl, 1,2-dimethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, cyclopropylmethyl, 1-cyclopropylethyl, 2-cyclopropylethyl, 1-cyclopropylpropyl, 2-cyclopropylpropyl, 3-cyclopropylpropyl, cyclobutylmethyl, 1-cyclobutylethyl, 2-cyclobutylethyl, cyclopentylmethyl, and the like. C₁-C₆ alkyl may preferably be methyl, ethyl, n-propyl, isopropyl, n-butyl, isobutyl, sec-butyl, tert-butyl, cyclopropyl, cyclobutyl, cyclopentyl, or cyclohexyl.

As used herein, the term “dialkylamino” means a group in which two hydrogen atoms in an amino group are each substituted with the same or different “alkyl” as defined above. In some embodiments, dialkylamino comprises N,N-dimethylamino, N,N-diethylamino, N,N-di-n-propylamino, N,N-di-isopropylamino, N,N-di-n-butylamino, N,N-di-isobutylamino, N,N-di-sec-butylamino, N,N-di-tert-butylamino, N-ethyl-N-methylamino, N-n-propyl-N-methylamino, N-isopropyl-N-methylamino, N-n-butyl-N-methylamino, N-isobutyl-N-methylamino, N-sec-butyl-N-methylamino, N-tert-butyl-N-methylamino, and the like. Dialkylamino may preferably be N,N-dimethylamino, N,N-diethylamino, or N-ethyl-N-methylamino. The term “di-C₁-C₄ alkylamino” as used herein means a group in which two hydrogen atoms in an amino group are each substituted with the same or different C₁-C₄ alkyl.

As used herein, the term “alkenyl” means a monovalent group derived from an alkene by removing one hydrogen atom. The term “Cₘ-Cₙ alkenyl” (where m and n are positive integers) as used herein means an alkenyl group having m to n carbon atoms. In some embodiments, alkenyl groups comprise vinyl, allyl, isopropenyl, 1-butenyl, 2-butenyl, 3-butenyl, and the like.

As used herein, the term “alkoxy” means a group in which an oxygen atom is bonded to the terminal of the “alkyl” as defined above. In some embodiments, alkoxy comprises methoxy, ethoxy, n-propoxy, isopropoxy, n-butoxy, isobutoxy, sec-butoxy, tert-butoxy, n-pentyloxy, isopentyloxy, sec-pentyloxy, neopentyloxy, 1-methylbutoxy, 2-methylbutoxy, 1,1-dimethylpropoxy, 1,2-dimethylpropoxy, n-hexyloxy, isohexyloxy, 1-methylpentyloxy, 2-methylpentyloxy, 3-methylpentyloxy, 1,1-dimethylbutoxy, 1,2-dimethylbutoxy, 2,2-dimethylbutoxy, 1,3-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy, and the like. Alkoxy may preferably be methoxy, ethoxy, n-propoxy, isopropoxy, n-butoxy, isobutoxy, sec-butoxy, or tert-butoxy. The term “Cₘ-Cₙ alkoxy” (where m and n are positive integers) as used herein means an alkoxy group having m to n carbon atoms.

As used herein, the term “alkylene” means a divalent group derived from an alkane by removing two hydrogen atoms. As used herein, the term “Cₘ-Cₙ alkylene” (where m and n are positive integers) means an alkylene having m to n carbon atoms. The alkylene may be linear or cyclic, preferably linear. Furthermore, the linear alkylene may be straight-chain or branched-chain, preferably straight-chain. In some embodiments, the alkylene may be a C₁-C₆ alkylene or a C₁-C₃ alkylene. The C₁-C₆ alkylene may be, for example, methylene, ethylene, n-propylene, n-butylene, n-pentylene, or n-hexylene. The C₁-C₃ alkylene may be, for example, methylene, ethylene, or n-propylene.

As used herein, the term “benzyl” means a monovalent group in which one hydrogen atom of a methyl group is substituted with a phenyl group. As used herein, the term “optionally substituted benzyl” means a benzyl group in which one or more hydrogen atoms are optionally substituted with substituents. In this case, the substituents may be, for example, alkoxy, halogen, hydroxy, carboxy, oxycarbonyl, amino, nitro, alkenyl, or alkyl. In some embodiments, in the optionally substituted benzyl, hydrogen atom(s) on the benzene ring and/or at the benzyl position may be substituted. In some embodiments, in the optionally substituted benzyl, hydrogen atom(s) on the benzene ring may be substituted, and the hydrogen atoms at the benzyl position are not substituted. In some embodiments, in the substituted benzyl, no hydrogen atom is substituted.

As used herein, the term “benzyloxy” means a monovalent group in which the hydrogen atom of a hydroxy group is substituted with a benzyl group. As used herein, the term “optionally substituted benzyloxy” means a monovalent group in which the hydrogen atom of a hydroxy group is substituted with an optionally substituted benzyl group.

As used herein, the term “linear or cyclic oligoalkylene glycol” in “a group obtained by removing one hydrogen atom from a hydroxyl group of a linear or cyclic oligoalkylene glycol” refers to a linear or cyclic oligomer of alkylene glycol. The linear oligoalkylene glycol may be straight-chain or branched-chain, and in one embodiment, it may be a straight-chain oligoalkylene glycol. Additionally, when the linear or cyclic oligoalkylene glycol is a cyclic oligoalkylene glycol, the group from which one hydrogen atom has been removed from a hydroxyl group of the cyclic oligoalkylene glycol may be, for example, a group in which one hydrogen atom of an alkylene contained in a ring composed of oligoalkylene glycol (e.g., cyclic oligoethylene glycol) is substituted with a hydroxyl group, and in which one hydrogen atom has been removed from the hydroxyl group or in which one hydrogen atom has been removed from a hydroxyl group contained in a linear oligoalkylene glycol further provided by forming an ether bond with the hydroxyl group.

At least one terminal of “linear or cyclic oligoalkylene glycol” in “a group obtained by removing one hydrogen atom from a hydroxyl group of a linear or cyclic oligoalkylene glycol” is hydroxyl group, and the other terminal(s) may be a hydroxyl group or an alkylated (e.g., methylated or ethylated) hydroxyl group, i.e., an alkoxy group.

Additionally, “linear or cyclic oligoalkylene glycol” in “a group obtained by removing one hydrogen atom from a hydroxyl group of a linear or cyclic oligoalkylene glycol” may be, for example, a linear or cyclic oligoalkylene glycol having 1 to 40 carbon atoms and 1 to 10 oxygen atoms, 1 to 30 carbon atoms and 1 to 10 oxygen atoms, 1 to 20 carbon atoms and 1 to 10 oxygen atoms, 1 to 15 carbon atoms and 1 to 10 oxygen atoms, 1 to 15 carbon atoms and 1 to 8 oxygen atoms, 1 to 15 carbon atoms and 1 to 6 oxygen atoms, 1 to 15 carbon atoms and 1 to 5 oxygen atoms, or 1 to 10 carbon atoms and 1 to 4 oxygen atoms. Furthermore, a polymerization degree of alkylene oxide monomer in “a linear or cyclic oligoalkylene glycol” in “a group obtained by removing one hydrogen atom from a hydroxyl group of a linear or cyclic oligoalkylene glycol” may, for example, be 1 to 20, 1 to 15, 1 to 12, 1 to 10, 1 to 8, 1 to 6, 1 to 5, 1 to 4, 1 to 3, 1 to 2, or 1.

The term “linear or cyclic oligoalkylene glycol” in “a group obtained by removing one hydrogen atom from a hydroxyl group of a linear or cyclic oligoalkylene glycol” comprises groups represented by R⁸—(O—R⁹—)ₚO—, in which R⁸ represents an alkyl group, R⁹ represents an alkylene group, and p represents an integer of 1 or more. One example of such a group comprises a group obtained by removing one hydrogen atom from a hydroxyl group of a linear oligoethylene glycol or a linear oligopropylene glycol, which may be alkylated (e.g., methylated or ethylated) at the terminal, specifically comprising methoxy-ethyleneoxy and methoxy-propyleneoxy. Additionally, the term “linear or cyclic oligoalkylene glycol” in “a group obtained by removing one hydrogen atom from a hydroxyl group of a linear or cyclic oligoalkylene glycol” comprises a group in which one hydrogen atom of an alkylene contained in a cyclic oligoethylene glycol or a cyclic oligopropylene glycol is substituted with a hydroxyl group, and one hydrogen atom has been removed from the hydroxyl group.

“A group in which one hydrogen atom on a carbon atom of a cyclic ether is replaced by an oxygen atom” is a monovalent group that forms a single bond with another group at the oxygen atom. The “cyclic ether” in “a group in which one hydrogen atom on a carbon atom of a cyclic ether is replaced by an oxygen atom” as used herein may be any cyclic ether, for example, a cyclic ether composed of carbon atoms, hydrogen atoms, oxygen atoms, and nitrogen atoms, or a cyclic ether composed of carbon atoms, hydrogen atoms, and oxygen atoms. The cyclic ether may be, for example, a cyclic ether in which one or more alkylene groups form a ring through an equal number of oxygen atoms, and the alkylene may be a linear alkylene in one embodiment. The cyclic ether may also be, for example, a monocyclic cyclic ether.

Furthermore, the “cyclic ether” in “a group in which one hydrogen atom on a carbon atom of a cyclic ether is replaced by an oxygen atom” may be, for example, a cyclic ether with 1 to 40 carbon atoms and 1 to 10 oxygen atoms, 1 to 30 carbon atoms and 1 to 10 oxygen atoms, 1 to 20 carbon atoms and 1 to 10 oxygen atoms, 1 to 15 carbon atoms and 1 to 10 oxygen atoms, 1 to 15 carbon atoms and 1 to 8 oxygen atoms, 1 to 15 carbon atoms and 1 to 6 oxygen atoms, 1 to 15 carbon atoms and 1 to 5 oxygen atoms, or 1 to 10 carbon atoms and 1 to 4 oxygen atoms. The cyclic part of the “cyclic ether” in “a group in which one hydrogen atom on a carbon atom of a cyclic ether is replaced by an oxygen atom” may be, for example, a cyclic part composed of 3 or more members, 4 or more members, or 5 or more members, and may be a cyclic part composed of 24 or fewer members, 18 or fewer members, 15 or fewer members, 12 or fewer members, 9 or fewer members, 8 or fewer members, 7 or fewer members, or 6 or fewer members. These upper and lower limits can be freely combined.

The “cyclic ether” in “a group in which one hydrogen atom on a carbon atom of a cyclic ether is replaced by an oxygen atom” may be, for example, ethylene oxide, oxetane, tetrahydrofuran, tetrahydropyran, 1,4-dioxane, hexamethylene oxide, 12-crown-4 ether, 15-crown-5 ether, or 18-crown-6 ether, in which one or more hydrogen atoms on the carbon atom may be substituted with alkyl, alkenyl, alkoxy, halogen, or hydroxy, and at least the oxygen atom in the crown ether may be substituted with NH. In one embodiment, the cyclic ether may be oxetane, tetrahydrofuran, tetrahydropyran, 1,4-dioxane, or 18-crown-6 ether, in another embodiment, it may be tetrahydrofuran or tetrahydropyran, and in yet another embodiment, it may be tetrahydrofuran.

The hydrogen atom(s) replaced in “a group in which one hydrogen atom on a carbon atom of a cyclic ether is replaced by an oxygen atom” may be, for example, a hydrogen atom on the carbon atom forming the cyclic part, and in one embodiment, it may be a hydrogen atom on the carbon atom forming an alkylene group contained in the cyclic part.

A first aspect of the present disclosure relates to a polynucleotide represented by the following formula (I):

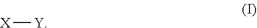

In formula (I), X is a polynucleotide comprising a 5′-cap structure at the 5′-end and a hydroxyl group bonded to the 3′-carbon of a nucleoside present at the 3′-end, which forms a phosphodiester bond integrally with the terminal of Y. The polynucleotide has a structure in which nucleotides are polymerized by phosphodiester bonds. In one embodiment, the polynucleotide regarding X may be a polymer in which all nucleotide monomers are bonded from the 5′-end to the 3′-end.

It should be noted that the “polynucleotide” and “oligonucleotide” as used herein are not limited to polymers in which all nucleotide monomers are bonded from the 5′-end to the 3′-end. For example, the “polynucleotide” and “oligonucleotide” may comprise polymers in which two or more polymers bonded from the 5′-end to the 3′-end form phosphodiester bonds at their respective 5′-ends and/or 3′-ends, or one or more polymers bonded from the 5′-end to the 3′-end and one or more nucleotide monomers form phosphodiester bonds at their respective 5′-ends and/or 3′-ends.

Furthermore, the “polynucleotide” and “oligonucleotide” as used herein are not limited to those in which the monomer units consist only of nucleotide monomers. For example, the 5′-end and/or 3′-end may have a structure not derived from nucleotides.

The nucleotide length of the polynucleotide regarding X according to one embodiment of the present disclosure is not particularly limited, but may be, for example, 30 mer or more, 50 mer or more, 100 mer or more, 300 mer or more, or 500 mer or more, and may be 1,000,000 mer or less, 300,000 mer or less, 100,000 mer or less, 70,000 mer or less, or 50,000 mer or less, and these upper limits and these lower limits can be freely combined. The nucleotide length of the polynucleotide regarding X according to one embodiment of the present disclosure may be, for example, 30 mer or more and 1,000,000 mer or less, 50 mer or more and 300,000 mer or less, 100 mer or more and 100,000 mer or less, 300 mer or more and 70,000 mer or less, or 500 mer or more and 50,000 mer or less.

The polynucleotide regarding X according to one embodiment of the present disclosure may have a polyadenyl region at the 3′-end. The polyadenyl region is a region where the proportion of adenosine monophosphate (5′-adenylic acid, adenosine phosphate, AMP) is high among the nucleotides constituting the polynucleotide. The polyadenyl region serves to inhibit the enzymatic degradation of the polynucleotide from the 3′-end side by nucleases or the like in vivo. Therefore, the polynucleotide regarding X according to one embodiment of the present disclosure, comprising a polyadenyl region at the 3′-end, enhances the stability of the polynucleotide in vivo.

The polyadenyl region may have a proportion of adenosine monophosphate among the nucleotides constituting the region in the polynucleotide regarding X, for example, 50% or more, 60% or more, 70% or more, 80% or more, 85% or more, 90% or more, 93% or more, 95% or more, 96% or more, 97% or more, 98% or more, or 99% or more, and may be 100% or less, 99% or less, 98% or less, 97% or less, 96% or less, or 95% or less, and these upper limits and these lower limits can be freely combined. The polyadenyl region may have a proportion of adenosine monophosphate among the nucleotides constituting the region in the polynucleotide regarding X, for example, 100%, 50% or more and 100% or less, 70% or more and 100% or less, 80% or more and 100% or less, 90% or more and 100% or less, 95% or more and 100% or less, 97% or more and 100% or less, 98% or more and 100% or less, or 99% or more and 100% or less. In a specific embodiment, the polyadenyl region may have a proportion of adenosine monophosphate among the nucleotides constituting the region in the polynucleotide regarding X of 100%. In other words, in a specific embodiment, the polyadenyl region may be a region consisting of a polymer of adenosine monophosphate. It should be noted that “the proportion of adenosine monophosphate among the nucleotides constituting the polyadenyl region in the polynucleotide regarding X is XX %” means that the region is a polymer consisting of XX % adenosine monophosphate and (100-XX) % of at least one kind of nucleotide other than adenosine monophosphate based on the number of nucleotides.

In the polyadenyl region, the nucleotides other than adenosine monophosphate contained in the nucleotides constituting the region in the polynucleotide regarding X are not particularly limited and may comprise natural or artificial nucleotides as constituent units, for example, at least one selected from guanosine monophosphate, uridine monophosphate, and cytidine monophosphate.

The nucleotide length of the polyadenyl region is not particularly limited, but may be, for example, 10 mer or more, 20 mer or more, 30 mer or more, 40 mer or more, or 50 mer or more, and may be 1,000 mer or less, 700 mer or less, 500 mer or less, 300 mer or less, or 200 mer or less, and these upper limits and these lower limits can be freely combined. The nucleotide length of the polyadenyl region according to one embodiment of the present disclosure may be, for example, 10 mer or more and 1,000 mer or less, 20 mer or more and 700 mer or less, 30 mer or more and 500 mer or less, 40 mer or more and 300 mer or less, or 50 mer or more and 200 mer or less.

A 5′-cap structure is a structure present at the 5′-end of mRNA in eukaryotes and exerts functions such as controlling the translation of mRNA and inhibiting enzymatic degradation. The 5′-cap structure in the polynucleotide according to the present disclosure may be a structure present in the mRNA of natural eukaryotic cells, or it may be an artificial analogue thereof. A 5′-cap structure commonly used by those skilled in the art can be utilized.

The 5′-cap structure may have a structure represented by the following formula. In the formula, the wavy line represents a bond to the 5′-end of X, R¹⁶ represents a natural or artificial nucleobase, and q represents an integer of 1 to 20.

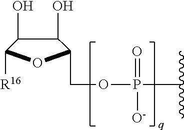

An example of the 5′-cap structure is a structure represented by the following formula. In the formula, the wavy line represents a bond to the 5′-end of X, q represents an integer of 1 to 5 or 2 to 4, and in a preferred embodiment, represents 3.

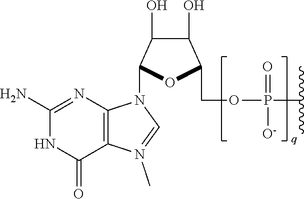

The 5′-cap structure may have a structure represented by the following formula. In the formula, the wavy line represents a bond to the 5′-end of X, and R¹⁷ and R¹⁸ each independently represent hydrogen atom or methyl.

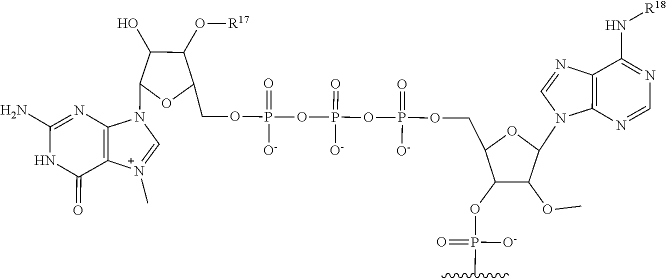

Such a 5′-cap structure can be introduced using, for example, the CleanCap™ Reagent series (TriLink BioTechnologies), specifically using CleanCap™ MAG, CleanCap™ AU, CleanCap™ AG3′OMe, or CleanCap™ M6, etc.

Furthermore, examples of the 5′-cap structure comprise those described in Patent Literature 2 (WO2023/282245), Patent Literature 3 (WO2021/162567), and Patent Literature 4 (WO2023/025073), which are incorporated herein by reference.

The polynucleotide regarding X according to one embodiment of the present disclosure may be a polyribonucleotide. In other words, X in the above formula (I) may be a polyribonucleotide comprising a 5′-cap structure at the 5′-end and a hydroxyl group bonded to the 3′-carbon of the nucleoside present at the 3′-end, which forms a phosphodiester bond integrally with the terminal of Y. In a preferred embodiment, X may be a polyribonucleotide comprising a 5′-cap structure at the 5′-end, a polyadenyl region at the 3′-end, and a hydroxyl group bonded to the 3′-carbon of the nucleoside present at the 3′-end, which forms a phosphodiester bond integrally with the terminal of Y.

The polyribonucleotide has a structure in which ribonucleotides are polymerized by phosphodiester bonds. The ribonucleotides constituting the polyribonucleotide may comprise naturally abundant ribonucleotides such as adenosine monophosphate, guanosine monophosphate, uridine monophosphate, and cytidine monophosphate, as well as artificial ribonucleotides that can be used as constituent units of mRNA in nucleic acid drugs containing mRNA. Examples of such artificial ribonucleotides comprise pseudouridine monophosphate, 2-thiouridine monophosphate, 6-methyladenosine monophosphate, 6-benzyladenosine monophosphate, inosinic acid (inosine monophosphate), 5-methylcytidine monophosphate, 1-methylpseudouridine monophosphate, and 5-hydroxymethylcytidine monophosphate. In one embodiment of the present disclosure, the ribonucleotide constituting the polyribonucleotide may be a nucleotide in which the hydroxyl group at the 1′-position of a ribose analogue selected from Group A is substituted with a nucleobase selected from Group B, a nucleotide in which the nucleobase in 2′-4′-LNA (2′-4′-Locked nucleic acid) is a nucleobase selected from Group B, or a nucleotide that is a monomer constituting PMO (phosphorodiamidate morpholino oligonucleotide) in which the nucleobase is a nucleobase selected from Group B.

Group A: ribose, 2′-deoxyribose, 2′-fluororibose, 2′-methoxyribose, 2′-(methoxy-ethylenoxy)ribose, 2′-4′-LNA, PMO
Group B: thymine, uracil, cytosine, adenine, guanine, 5-methylcytosine, 6-methyladenine, 6-benzyladenine, 1-methyluracil, 5-hydroxymethylcytosine, 2-thiouracil, hypoxanthine

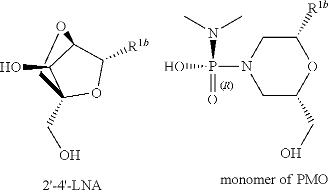

The polyribonucleotide according to one embodiment of the present disclosure comprises a coding region that encodes an amino acid sequence of a translated protein. The coding region typically starts with a start codon and ends with a stop codon, and such a region is also known as an open reading frame (ORF). The nucleotide length of the coding region is not particularly limited and can be appropriately set according to the translated protein. The nucleotide length of the coding region may be, for example, 6 mer or more, 10 mer or more, 15 mer or more, 20 mer or more, 30 mer or more, 50 mer or more, 100 mer or more, 300 mer or more, or 500 mer or more, and may be 1,000,000 mer or less, 300,000 mer or less, 100,000 mer or less, 70,000 mer or less, or 50,000 mer or less, and these upper limits and these lower limits can be freely combined. The nucleotide length of the coding region according to one embodiment of the present disclosure may be, for example, 6 mer or more and 1,000,000 mer or less, 15 mer or more and 300,000 mer or less, 30 mer or more and 100,000 mer or less, 100 mer or more and 70,000 mer or less, or 500 mer or more and 50,000 mer or less.

The polyribonucleotide according to one embodiment of the present disclosure may further comprise a 5′-Untranslated region (5′-UTR). The 5′-UTR is present at the 5′-end of mRNA and is not translated during the process of protein synthesis, and instead plays a role in regulating the translation of the protein encoded by the coding region. The 5′-UTR may comprise, for example, a Kozak sequence.

The polyribonucleotide according to one embodiment of the present disclosure may further comprise a 3′-Untranslated region (3′-UTR). The 3′-UTR is present at the 3′-end of mRNA before polyadenylation and is not translated during the process of protein synthesis, and instead plays a role in regulating the translation of the protein encoded by the coding region and enhancing the stability of mRNA in vivo. In vivo, the presence of the 3′-UTR allows the formation of a poly(A) tail at the 3′-end by poly(A) polymerase.

The polyribonucleotide according to one embodiment of the present disclosure may comprise the 5′-UTR, coding region, and 3′-UTR in this order. In other words, the polynucleotide regarding X according to one embodiment of the present disclosure may be a polyribonucleotide comprising a 5′-cap structure at the 5′-end, a hydroxyl group bonded to the 3′-carbon of the nucleoside present at the 3′-end, which forms a phosphodiester bond integrally with the terminal of Y, and comprising the 5′-UTR, coding region, and 3′-UTR in this order. Furthermore, the polynucleotide regarding X according to one embodiment of the present disclosure may be a polyribonucleotide comprising a 5′-cap structure at the 5′-end, a hydroxyl group bonded to the 3′-carbon of the nucleoside present at the 3′-end, which forms a phosphodiester bond integrally with the terminal of Y, and comprising the 5′-UTR, coding region, 3′-UTR, and polyadenyl region in this order. It should be noted that when the order of the components of the polynucleotide is specified above, the order is from the 5′-end to the 3′-end.

In formula (I), Y is represented by the following formula (II-A):

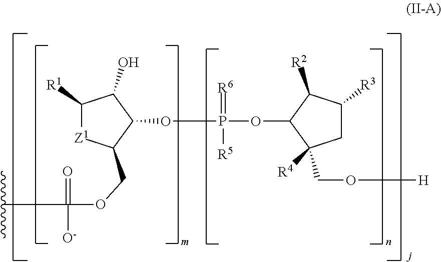

the following formula (III):

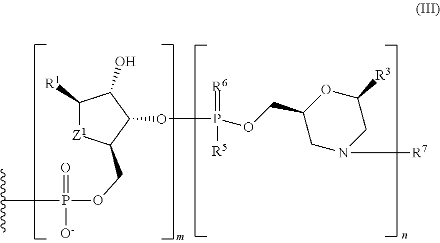

the following formula (IV):

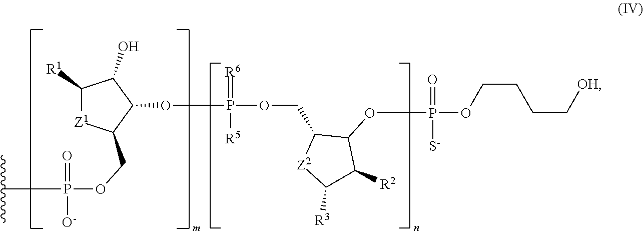

or the following formula (V).

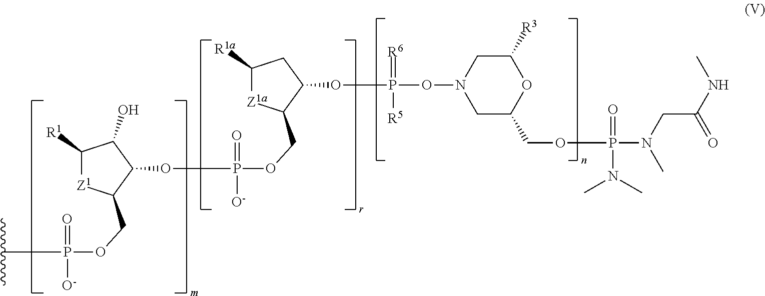

In one embodiment of the present disclosure, Y in formula (I) is represented by the following formula (II′-A):

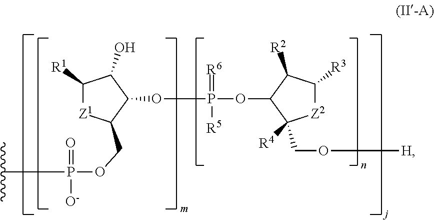

or the following formula (II′-AX).

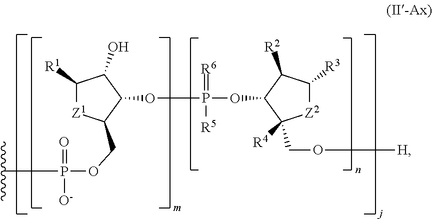

In formulas (II-A), (II′-A), (IT′-AX), (III), (IV), and (V), the wavy line represents a bond to X.

In a preferred embodiment of the present disclosure, j in the above formula (II-A) is 1. That is, in formula (I), Y is represented by the following formula (II):

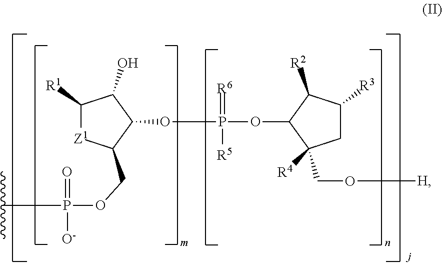

the following formula (III):

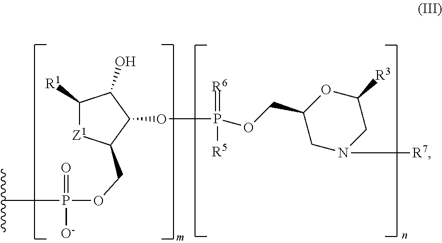

the following formula (IV):

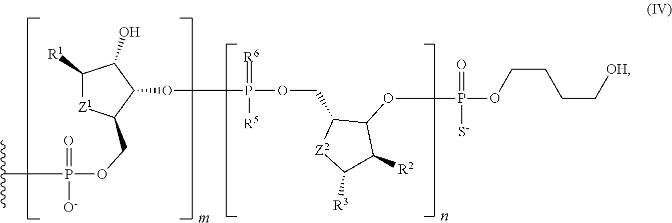

or the following formula (V).

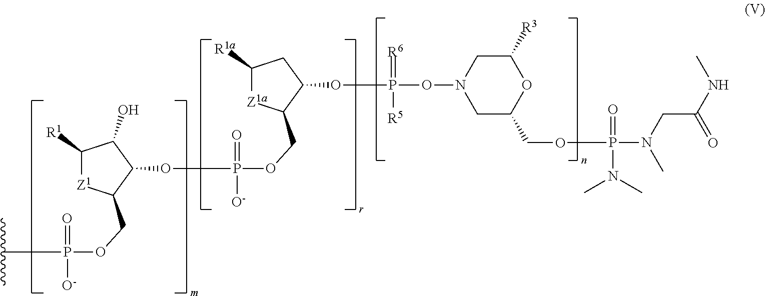

In a preferred embodiment of the present disclosure, j in the above formula (II′-A) or (II′-AX) is 1. That is, in a preferred embodiment of the present disclosure, Y in formula (I) is represented by the following formula (II′):

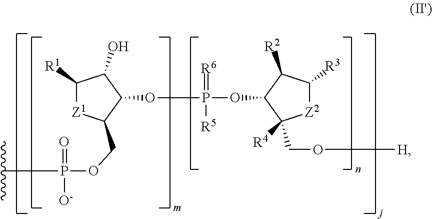

or the following formula (II′-X).

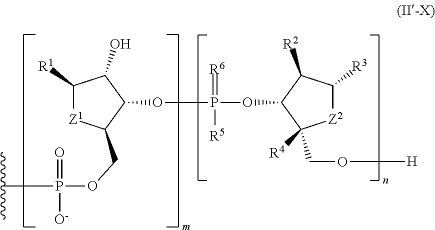

In formulas (II), (II′), (IT′-X), (III), (IV), and (V), the wavy line represents a bond to X.

In formulas (II), (II′), (II′-X), (II-A), (II′-A), (II′-AX), (III), (IV), and (V), R¹, R^{1a}, and R³ each independently represent a nucleobase. When Y has two or more R¹ groups, each of the two or more R¹ groups independently represents a nucleobase, which may be the same or different nucleobases. When Y has two or more R^{1a} groups, each of the two or more R^{1a} groups independently represents a nucleobase, which may be the same or different nucleobases, or may represent the same nucleobase. When Y has two or more R³ groups, each of the two or more R³ groups independently represents a nucleobase, which may be the same or different nucleobases, or may represent the same nucleobase. The nucleobase may be a natural base such as adenine, cytosine, guanine, uracil, or thymine, or it may be an artificial nucleobase that can be used as a constituent in nucleic acid drugs containing polynucleotides such as mRNA. Examples of such artificial nucleobases comprise 5-methylcytosine, 6-methyladenine, 6-benzyladenine, 1-methyluracil, 5-hydroxymethylcytosine, 2-thiouracil, or hypoxanthine.

The nucleobase mentioned above may be, for example, thymine, uracil, cytosine, adenine, guanine, 5-methylcytosine, 6-methyladenine, 6-benzyladenine, 1-methyluracil, 5-hydroxymethylcytosine, 2-thiouracil, or hypoxanthine, and may also be adenine, cytosine, guanine, uracil, thymine, or 5-methylcytosine. In other words, R¹, R^{1a}, and R³ may be, for example, thymine, uracil, cytosine, adenine, guanine, 5-methylcytosine, 6-methyladenine, 6-benzyladenine, 1-methyluracil, 5-hydroxymethylcytosine, 2-thiouracil, or hypoxanthine, and may also be adenine, cytosine, guanine, uracil, thymine, or 5-methylcytosine. R¹, R^{1a}, and R³ may be a group represented by a formula selected from the following group:

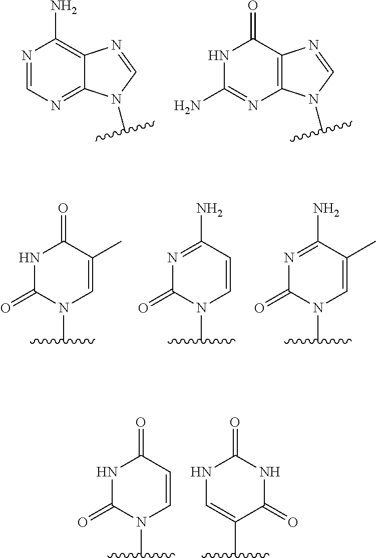

In one embodiment of the present disclosure, R¹ may be adenine, guanine, cytosine, thymine, or uracil, and R³ may be thymine, uracil, cytosine, adenine, guanine, or 5-methylcytosine. In one embodiment of the present disclosure, R¹ and R^{1a} may be adenine, cytosine, guanine, or uracil, and R³ may be thymine, adenine, cytosine, guanine, uracil, or 5-methylcytosine. In one embodiment of the present disclosure, R¹ may be adenine, cytosine, or uracil, and R³ may be thymine, uracil, cytosine, adenine, guanine, or 5-methylcytosine. In one embodiment of the present disclosure, R¹ and R^{1a} may be a group represented by a formula selected from the following group:

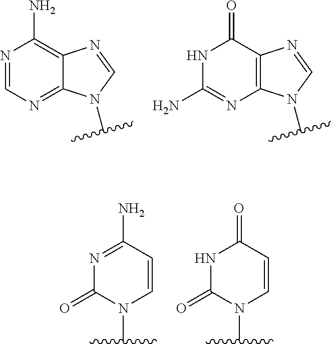

and R³ may be a group represented by a formula selected from the following group:

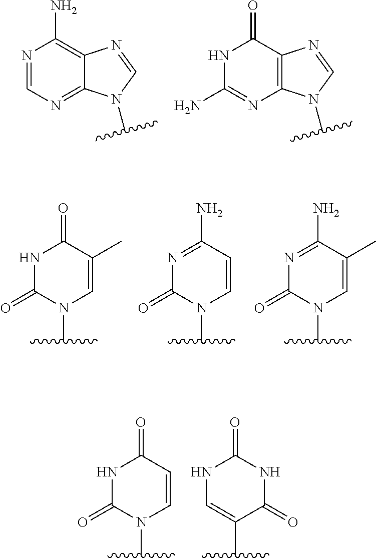

In one embodiment of the present disclosure, R¹ and R^{1a} may be adenine, and R³ may be thymine, adenine, cytosine, guanine, uracil, or 5-methylcytosine. In one embodiment of the present disclosure, R¹ and R^{1a} may be a group represented by the following formula:

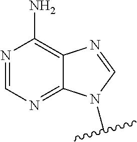 [SMILES: *n1cnc2c(N)ncnc21]

and R³ may be a group represented by a formula selected from the following group:

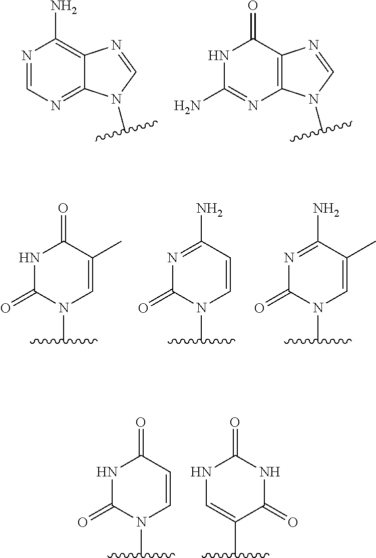

In one preferred embodiment of the present disclosure, R¹ and R^{1a} may be adenine, and R³ may be thymine. In one preferred embodiment of the present disclosure, R¹ and R^{1a} may be a group represented by the following formula:

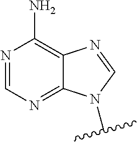 [SMILES: *n1cnc2c(N)ncnc21]

and R³ may be a group represented by the following formula:

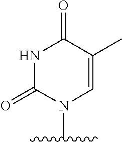 [SMILES: *n1cc(C)c(=O)[nH]c1=O]

In formulas (II), (II′), (IT′-X), (II-A), (II′-A), (II′-AX), (III), (IV), and (V), R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II-A), formula (II′-A), formula (II′-AX), formula (II), formula (II′), formula (II′-X), or formula (IV), and R⁴ represents H or together with R² represents the group represented by —O—R¹⁰—.

In one embodiment, R² represents a group obtained by removing one hydrogen atom from a hydroxyl group of a linear or cyclic oligoalkylene glycol having 1 to 40 carbon atoms and 1 to 10 oxygen atoms, a group in which one hydrogen atom on a carbon atom of a cyclic ether having 1 to 40 carbon atoms and 1 to 10 oxygen atoms is replaced by an oxygen atom, a C₁-C₃₀ alkoxy, an optionally substituted benzyloxy, a halogen, a hydroxy, a C₂-C₁₀ alkenyl, or a C₁-C₁₀ alkyl; or together with R⁴, represents a group represented by —O—R¹⁰—, wherein R¹⁰ represents a C₁-C₃ alkylene, and wherein the oxygen atom in the group forms a single bond with the carbon atom to which R² is bonded in formula (II-A), formula (II′-A), formula (II′-AX), formula (II), formula (II′), formula (II′-X), or formula (IV), and R⁴ represents H or together with R² represents the group represented by —O—R¹⁰—.
In another embodiment, R² represents a group obtained by removing one hydrogen atom from a hydroxyl group of a linear or cyclic oligoalkylene glycol having 1 to 15 carbon atoms and 1 to 10 oxygen atoms, a group in which one hydrogen atom on a carbon atom of a cyclic ether having 1 to 15 carbon atoms and 1 to 10 oxygen atoms is replaced by an oxygen atom, a C₁-C₂₀ alkoxy, a benzyloxy, fluoro, a C₂-C₁₀ alkenyl, or a C₁-C₁₀ alkyl, and R⁴ represents H.
In another embodiment, R² represents a group represented by CH₃—(O—CH₂—CH₂—)ₚO— or (CH₃O(—CH₂—CH₂—O—)_{w}CH₂—)₂CH—O—, in which p represents an integer of 1 to 2 and w represents an integer of 0 to 2; a group in which one hydrogen atom on a carbon atom of tetrahydrofuran or tetrahydropyran is replaced by an oxygen atom; a C₁-C₁₆ alkoxy; a benzyloxy; a C₂-C₅ alkenyl; or a C₁-C₅ alkyl, and R⁴ represents H.
In a preferred embodiment, R² is selected from the group consisting of methoxy-ethyleneoxy, methoxy, a benzyloxy, allyl, and a group represented by the following formulas:

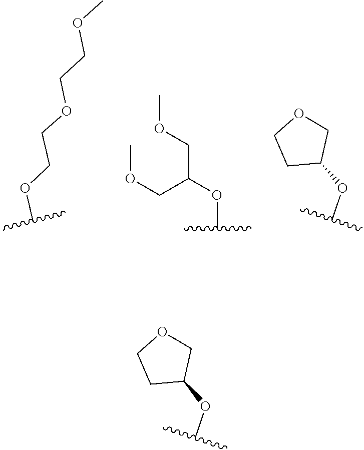

and R⁴ represents H.
In a more preferred embodiment, R² represents methoxy-ethyleneoxy, and R⁴ represents H.
It should be noted that in the present disclosure, “the carbon atom to which R² is bonded in a formula (II), (II′), (II′-X), (II-A), (IT′-A), (II′-AX), or (IV)” refers to a carbon atom indicated by * in the following formulas:

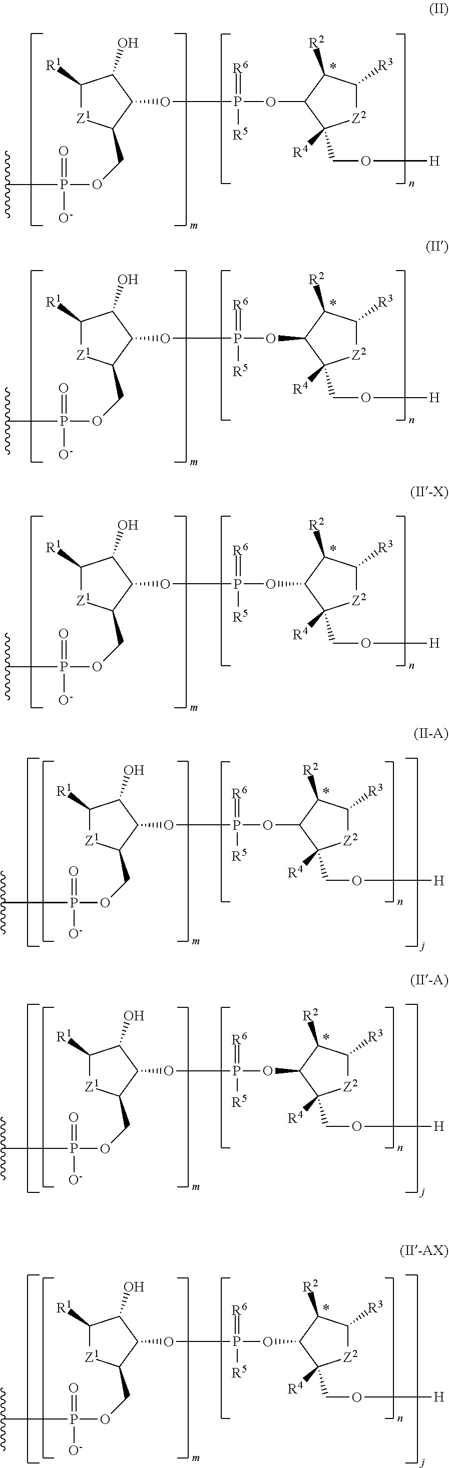

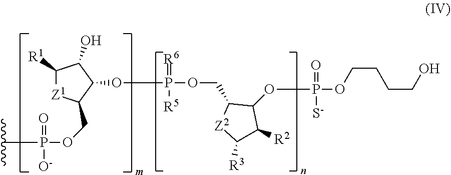

In formulas (II), (II′), (IT′-X), (II-A), (IT′-A), (II′-AX), (III), (IV), and (V), R⁵ may represent O⁻, S⁻, BH₃⁻, Se⁻, or dialkylamino. In one embodiment, R⁵ in formulas (II), (II′), (II′-X), (II-A), (II′-A), (II′-AX), (III), and (IV) may represent O⁻, S⁻, BH₃⁻, or Se⁻, and may represent O⁻ or S⁻, and in a preferred embodiment, R⁵ in formulas (II), (II′), (II′-X), (II-A), (II′-A), (II′-AX), (III), and (IV) may represent O⁻. In one embodiment, R⁵ in formula (V) may represent dialkylamino, may represent di-C₁-C₄-alkylamino, may represent dimethylamino or diethylamino, or may represent dimethylamino.

In formulas (II), (II′), (II′-X), (II-A), (II′-A), (IT′-AX), (III), (IV), and (V), R⁶ represents O, S, or Se. In one embodiment, R⁶ may represent O or S. In a preferred embodiment, R⁶ may represent O.

In formula (III), R⁷ may represent H or a group represented by R¹¹—C(═O)—, wherein R¹¹ represents alkyl. In one embodiment, R⁷ may represent H or the group where R¹¹ is C₁-C₁₀ alkyl, in another embodiment, R⁷ may represent H or the group where R¹¹ is C₁-C₆ alkyl, in another embodiment, R⁷ may represent H or the group where R¹¹ is C₁-C₄ alkyl, and in another embodiment, R⁷ may represent H or acetyl.

In formulas (II), (II′), (II′-X), (II-A), (II′-A), (II′-AX), (III), (IV), and (V), Z¹, Z^{1a}, and Z² each independently represent O, S, Se, NR¹², or CR¹³R¹⁴ wherein R¹², R¹³, and R¹⁴ each independently represent alkyl or alkenyl. In one embodiment, R¹², R¹³, and R¹⁴ each independently represent C₁-C₄ alkyl or C₂-C₄ alkenyl. In another embodiment, R¹², R¹³, and R¹⁴ each independently represent methyl, ethyl, propyl, vinyl, or allyl. In another embodiment, R¹², R¹³, and R¹⁴ each independently represent methyl.

In one embodiment, Z¹, Z^{1a}, and Z² each independently represent O, S, or Se. In one embodiment, Z¹, Z^{1a}, and Z² each independently represent O or S. In a preferred embodiment, Z¹, Z^{1a}, and Z² represent O.

In formulas (II-A), (II′-A), and (II′-AX), j may represent an integer of 1 to 10, 1 to 5, 1 to 4, 1 to 3, or 1 to 2, or may represent 1. In a preferred embodiment, j may represent 1 or 2, or may represent 1.

In formulas (II), (II′), (II′-X), (II-A), (II′-A), (II′-AX), (II), (IV), and (V), m and n each independently represent an integer of 1 to 50. In one embodiment, m and n each independently represent an integer of 1 to 20, 1 to 15, 1 to 10, 1 to 7, 1 to 5, 1 to 4, 1 to 3, or 1 to 2. In a preferred embodiment, m may represent an integer of 1 to 10. In a preferred embodiment, n may represent 1 or 2, or may represent 1.

In formula (V), r may represent an integer of 0 to 20. In one embodiment, r may represent an integer of 0 to 15, 0 to 10, 0 to 7, 0 to 5, 0 to 4, 0 to 3, 0 to 2, or 0 to 1. In one embodiment, r may be 0 or 1.

In one embodiment, Y in the above formula (I) may be, for example, a group represented by a formula selected from the group consisting of the following formulas.

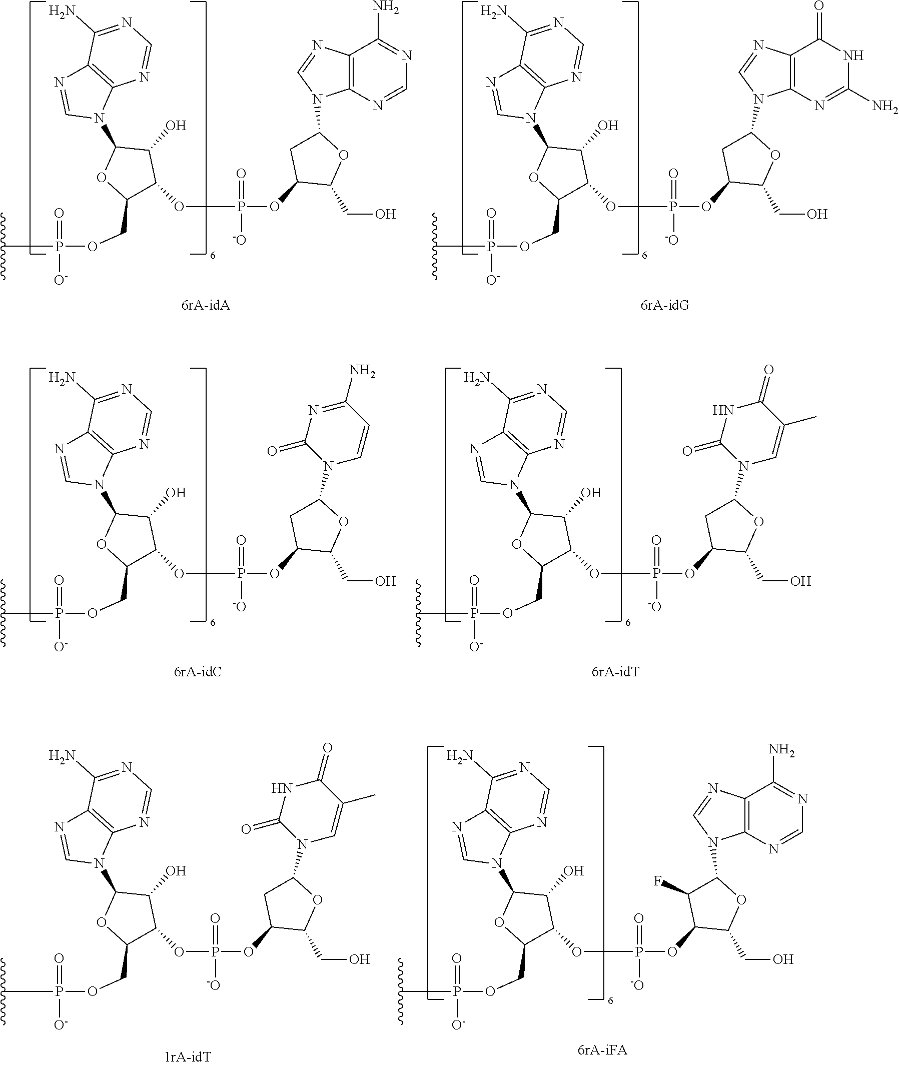

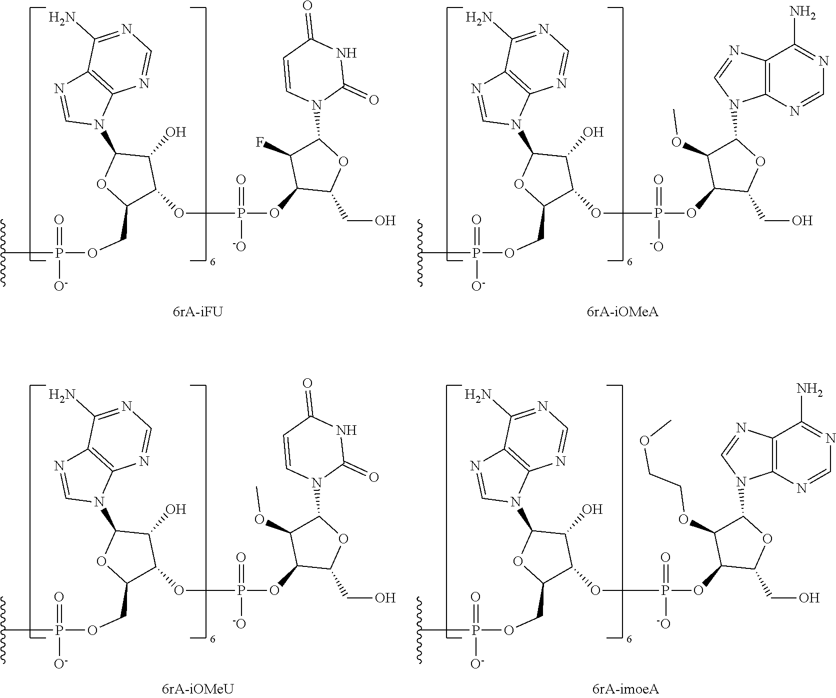

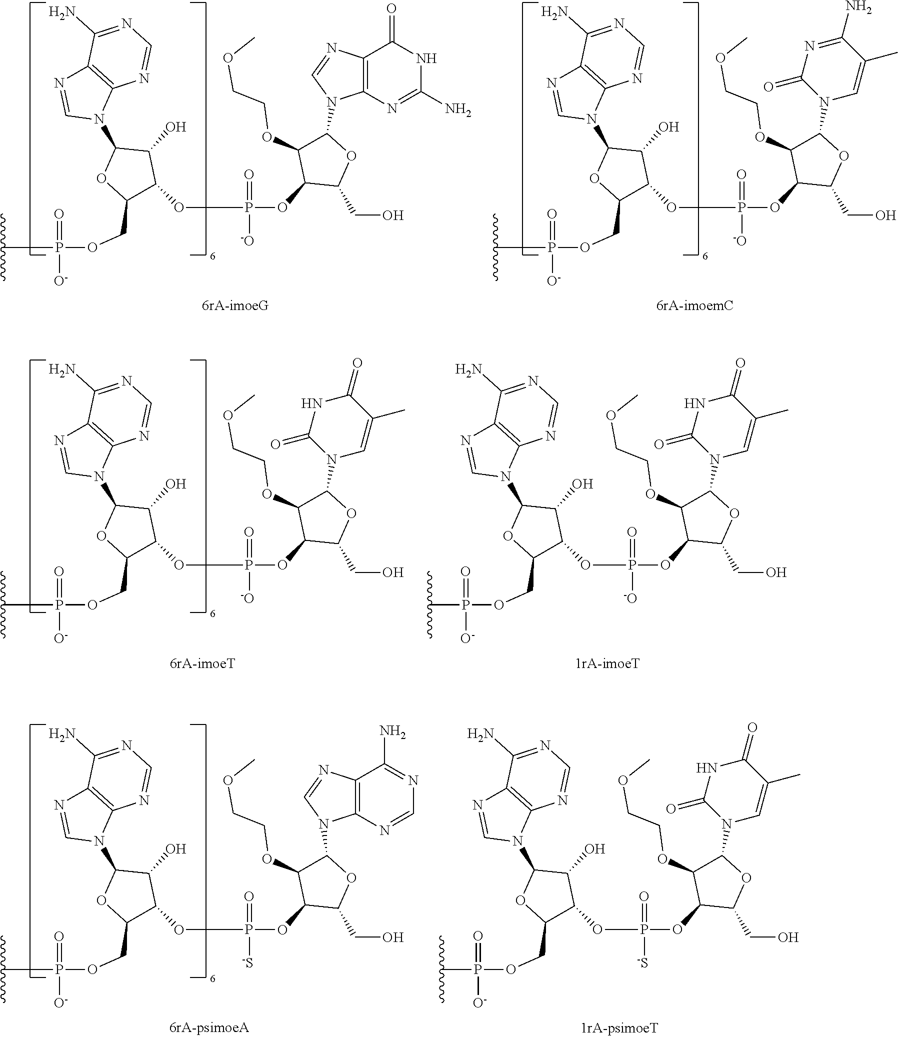

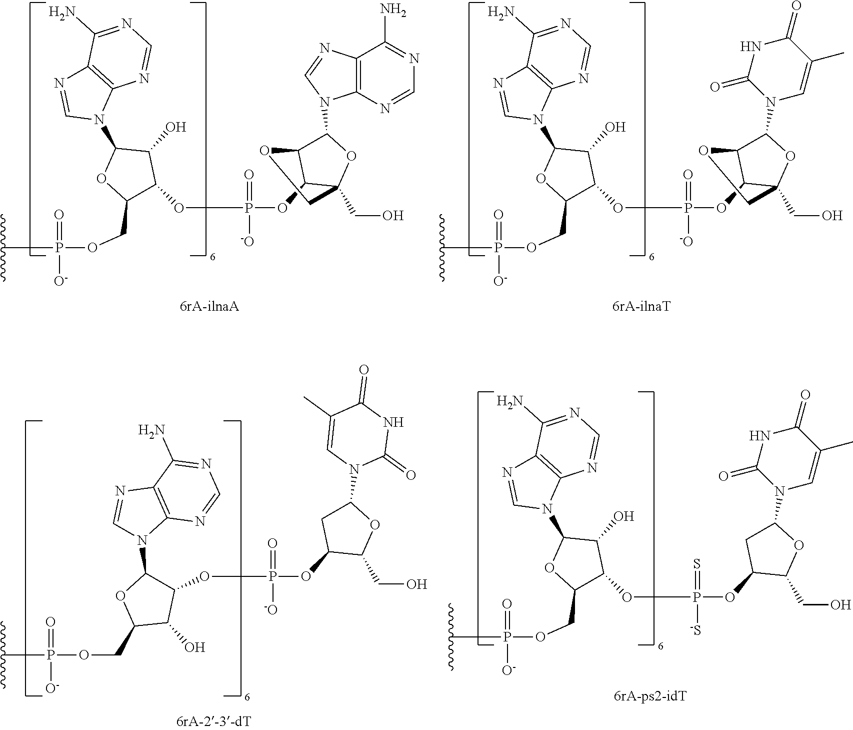

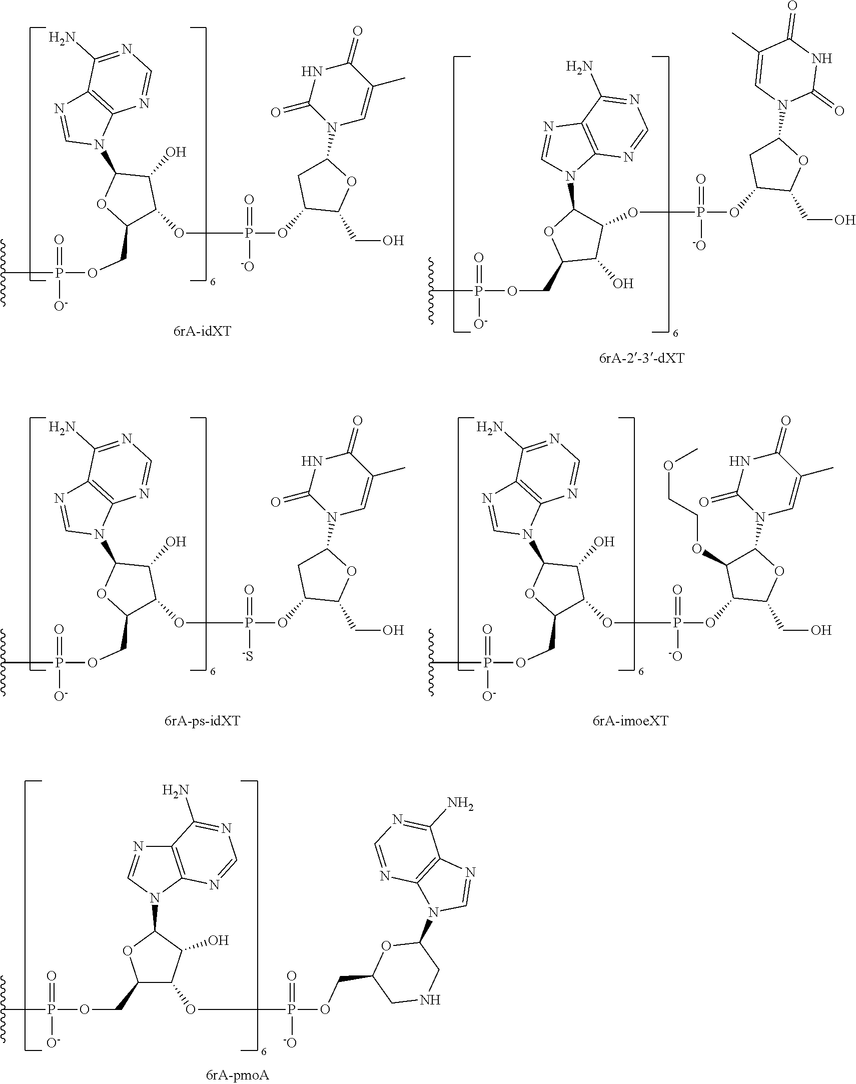

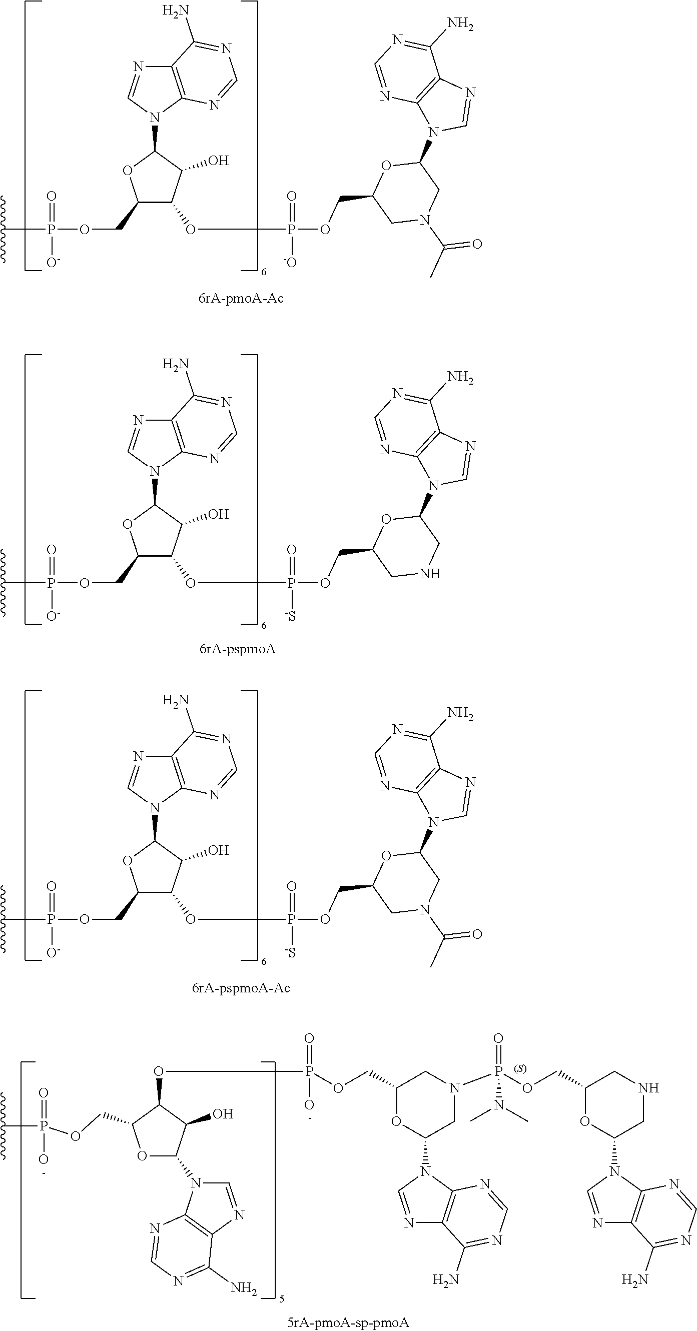

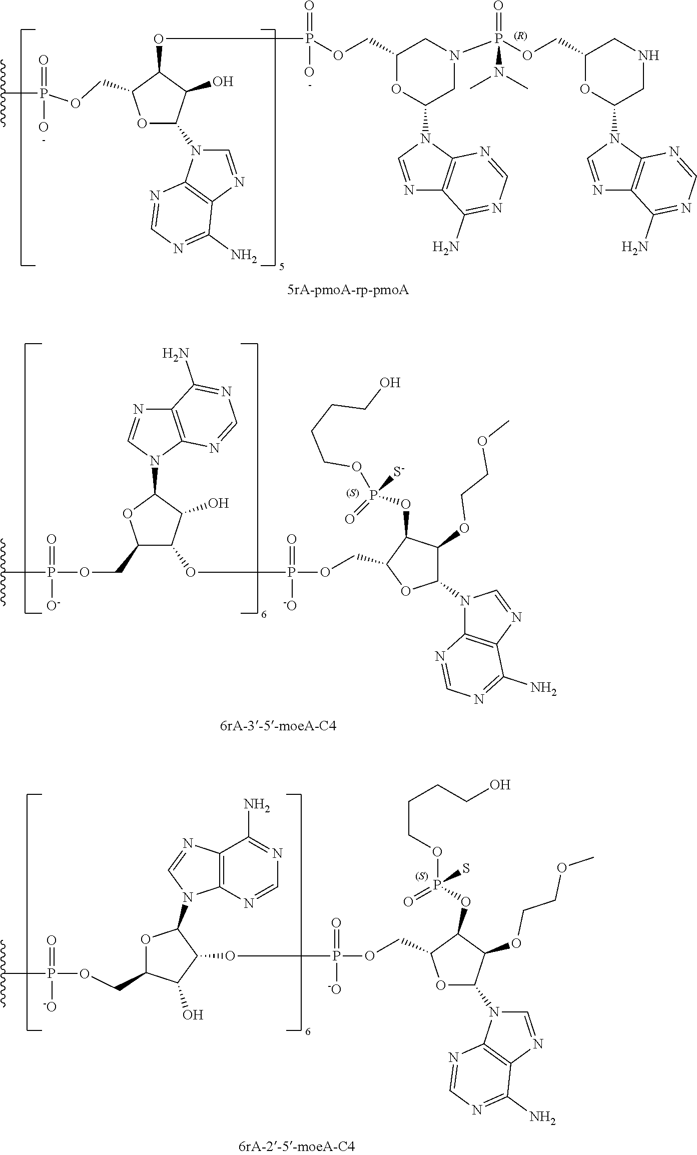

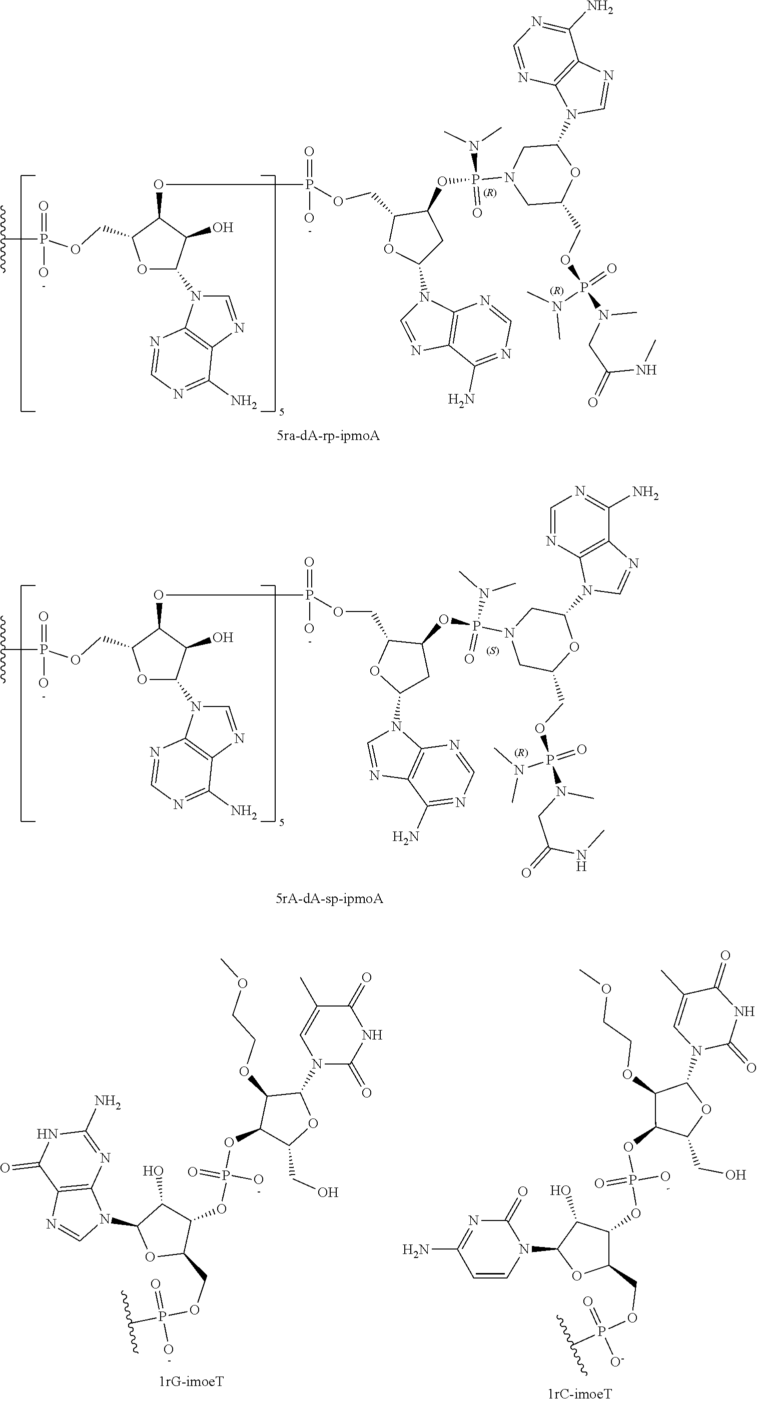

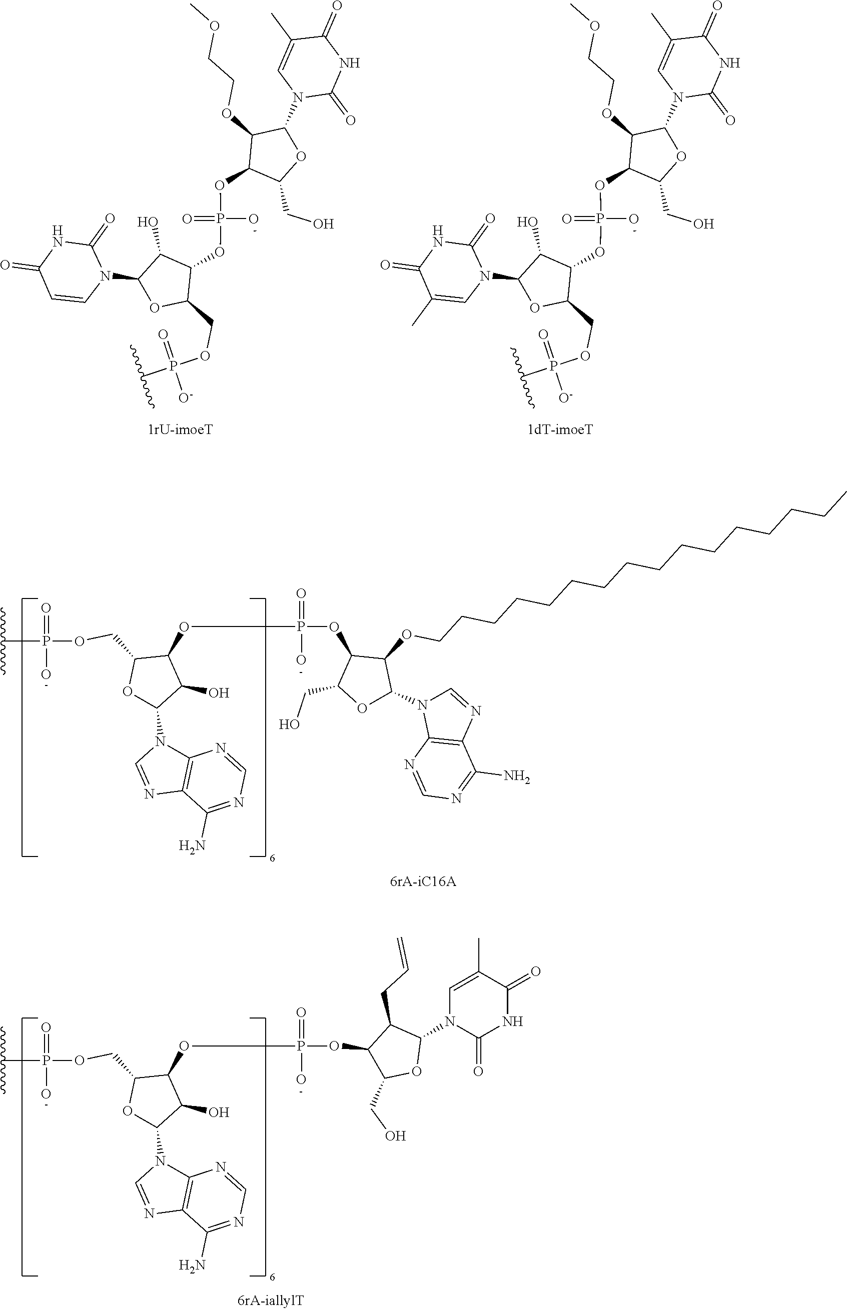

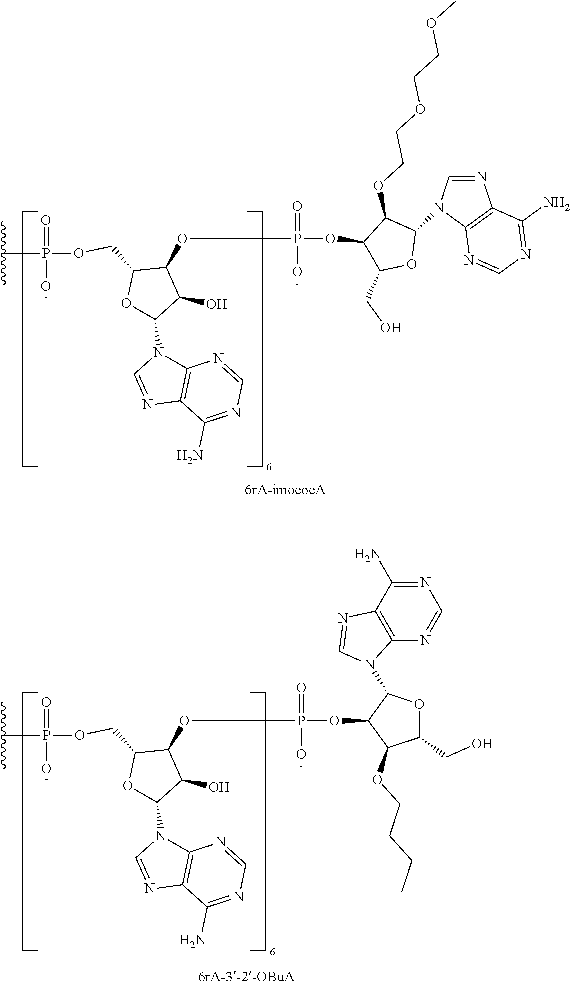

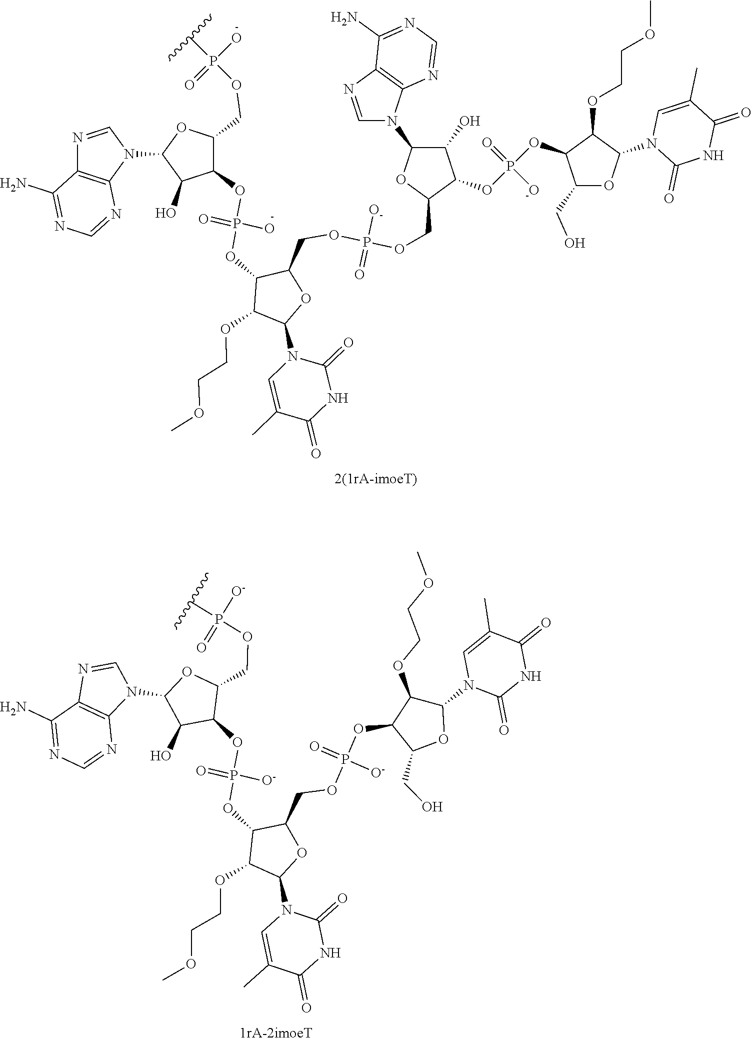

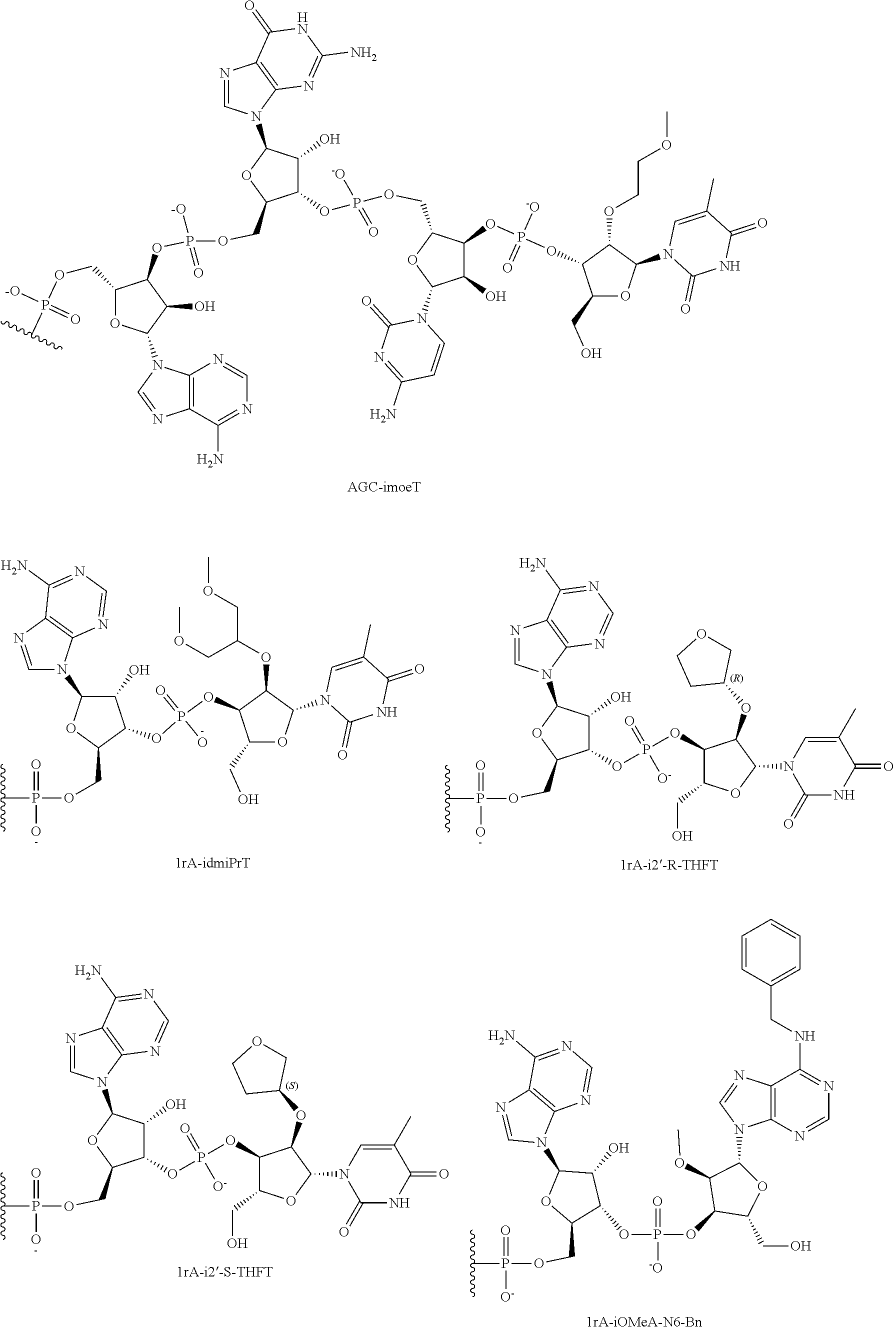

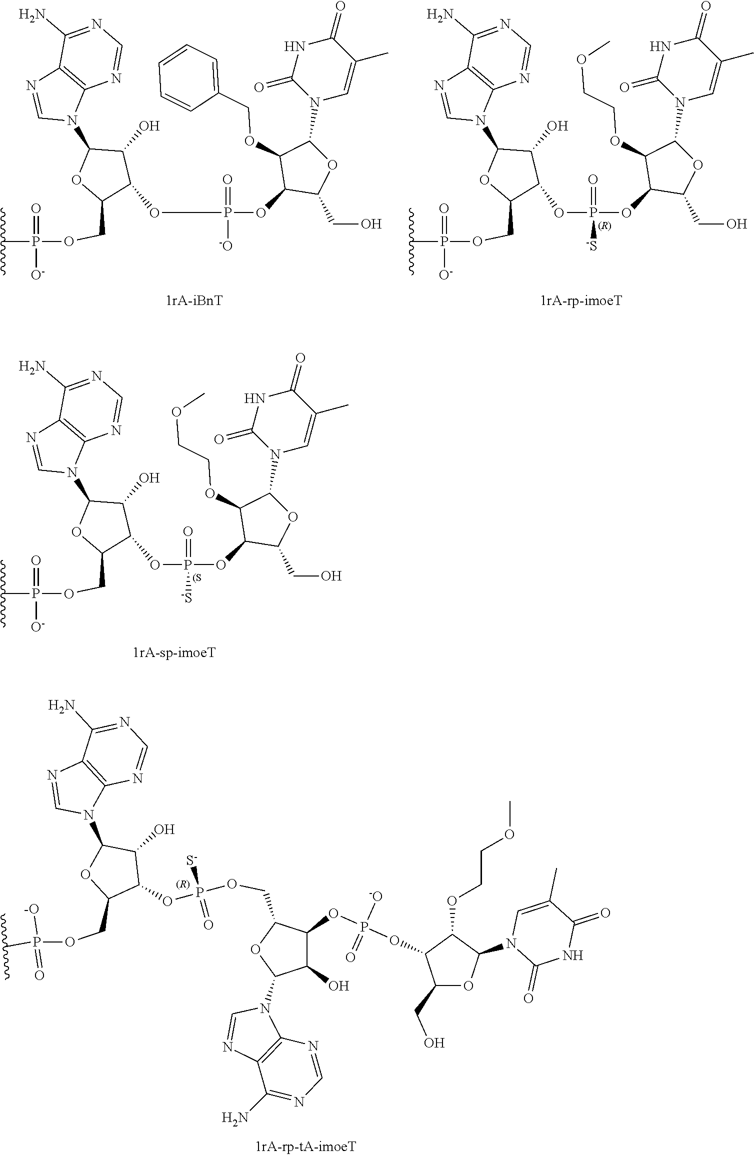

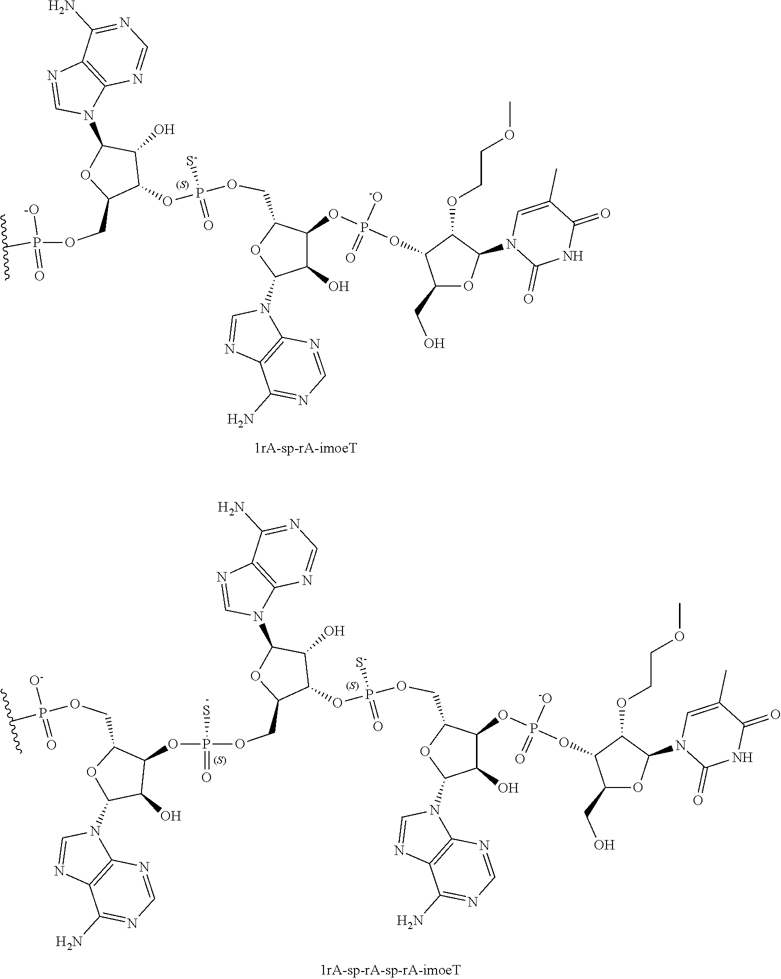

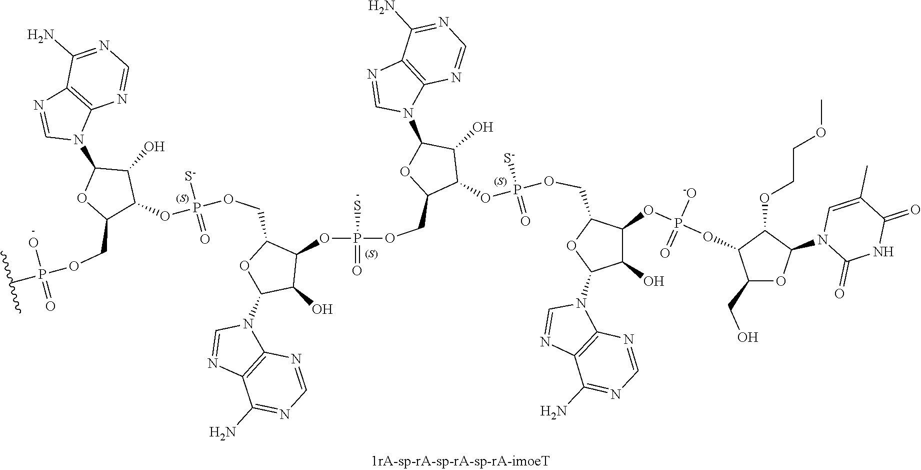

In a preferred embodiment, Y in the above formula (I) may be, for example, a group represented by a formula selected from the group consisting of the following formulas.

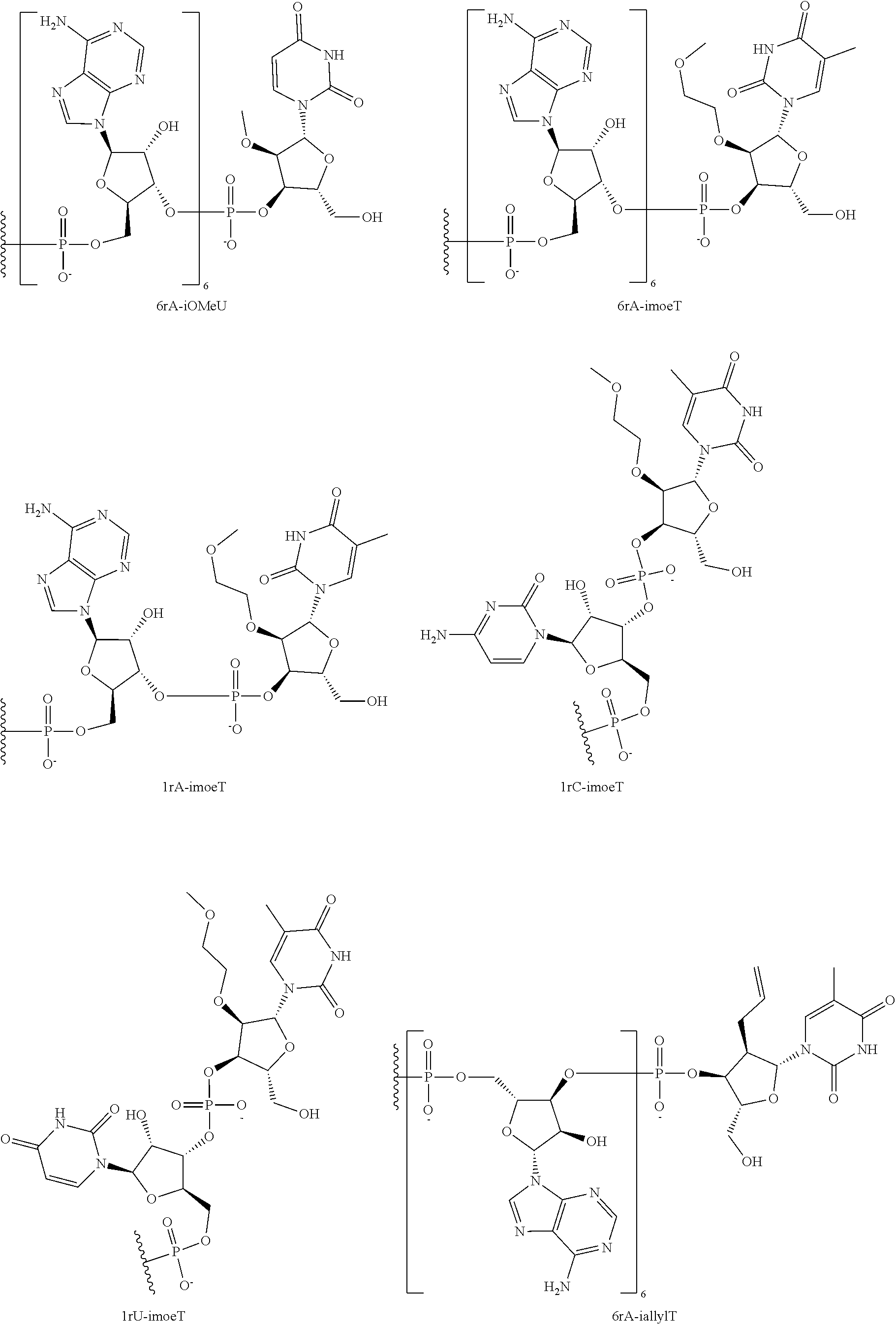

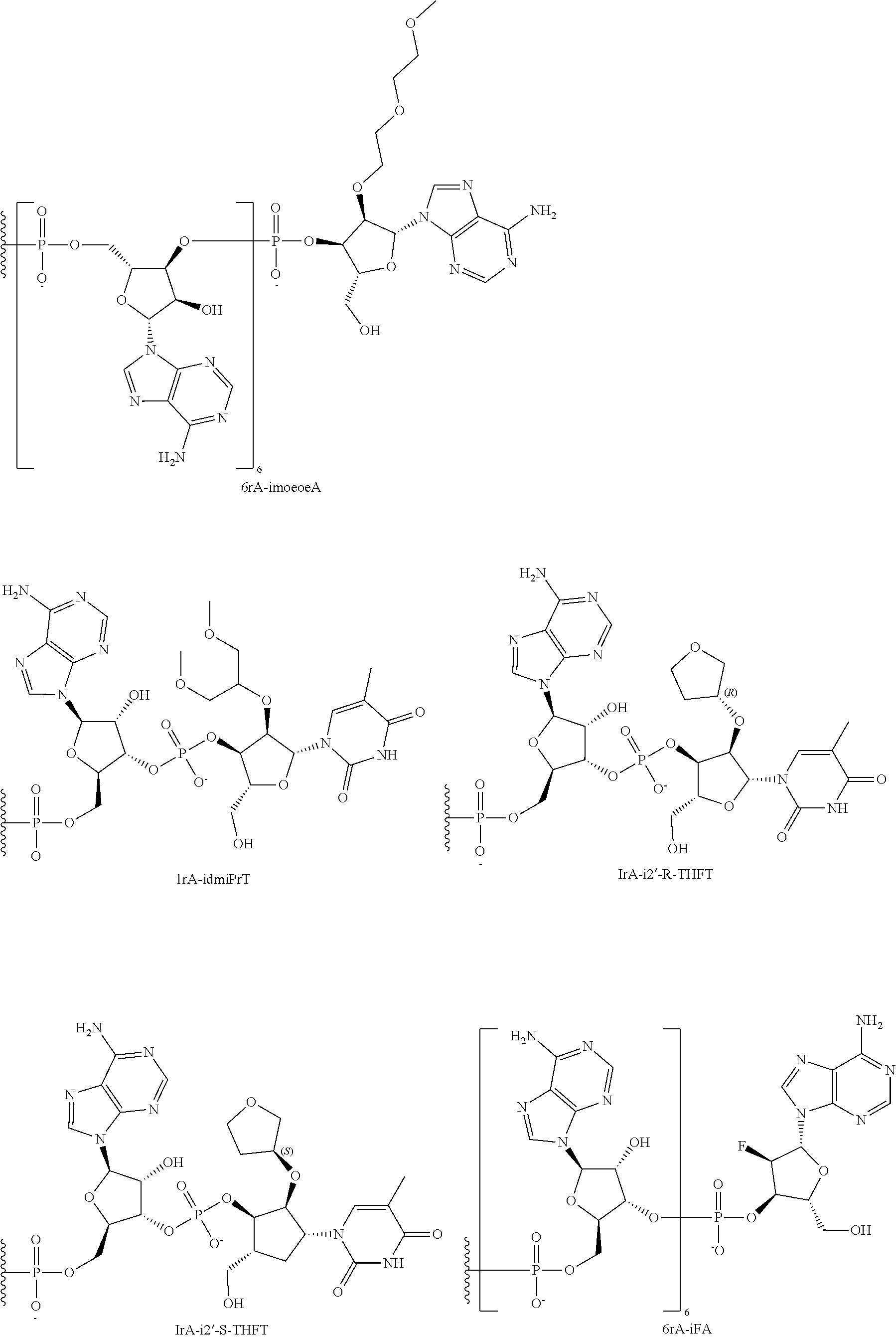

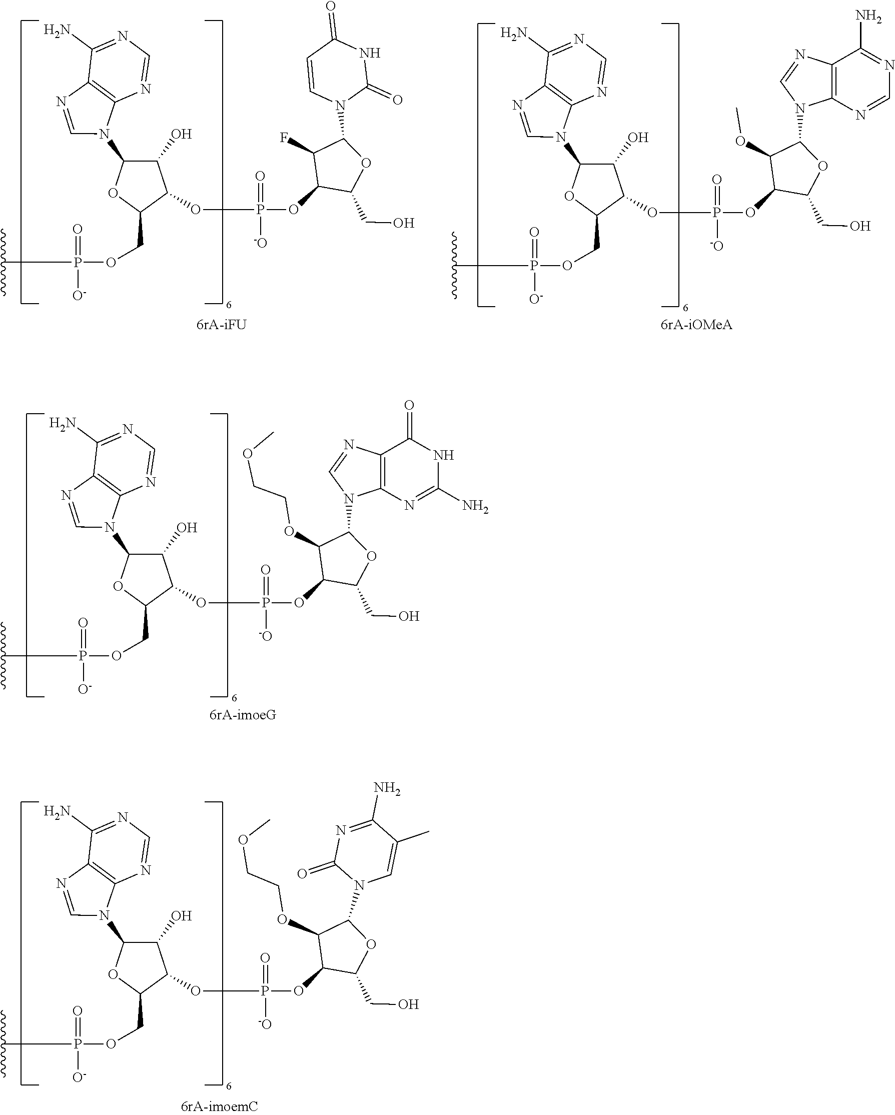

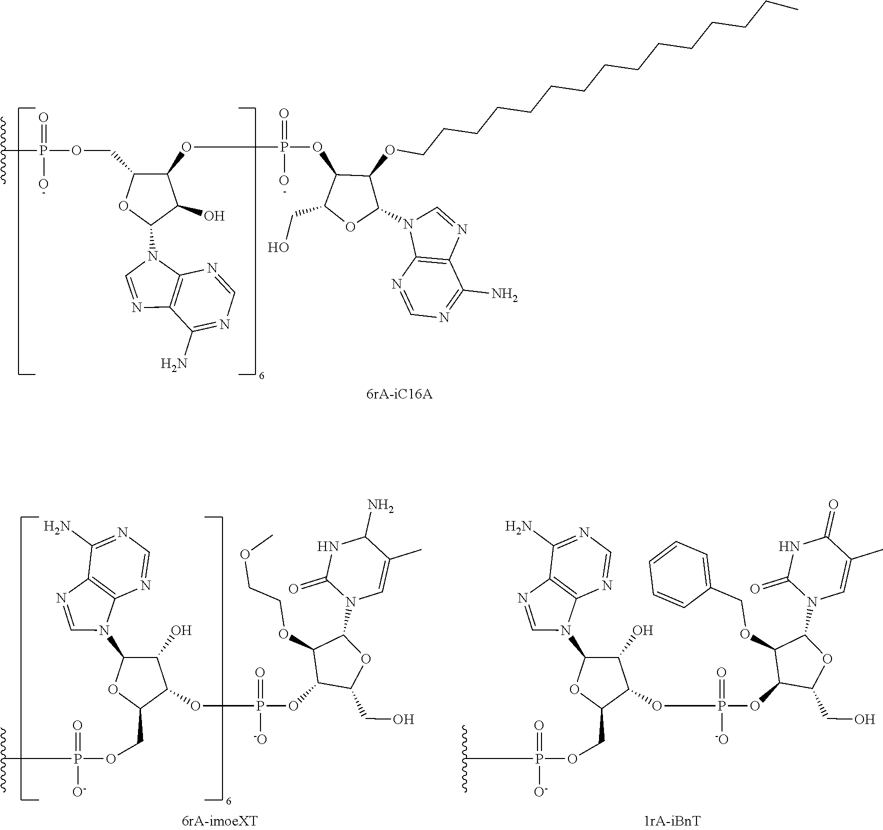

In the present disclosure, rA means adenosine, and “6rA” or “1rA” means a polyribonucleotide formed by six or one adenosine monophosphate (AMP), respectively. Additionally, in the present disclosure, “i” in a chemical name indicates that the 3′-hydroxyl group of ribose in the nucleoside forms a phosphodiester bond with the 3′-hydroxyl group of ribose in another nucleoside via a phosphorus atom. On the other hand, “X” in a chemical name indicates that the configuration of the hydroxyl group and hydrogen atom at the 3′-position of ribose in the nucleoside is reversed compared to normal ribose (i.e., it becomes xylose). Furthermore, “ps (PS)” in the present disclosure indicates that the oxygen atom bonded to the phosphorus atom in the phosphodiester bond is replaced by a sulfur atom. In the present disclosure, “rp” and “sp” indicate that the stereochemical configuration of the substituents on the phosphorus atom is R and S, respectively. Additionally, “2′-R” and “2′-S” indicate that the stereochemical configuration of the carbon atom substituting the hydrogen atom in the hydroxyl group bonded to the 2′-carbon atom of ribose is R and S, respectively. In the present disclosure, “-<number A>-<number B>-” in a chemical name indicates that the hydroxyl group at the position indicated by number A in the ribose part of the nucleoside abbreviated before number A and the hydroxyl group at the position indicated by number B in the ribose part of the nucleoside abbreviated after number B form a phosphodiester bond via a phosphorus atom. Similarly, “<number B>-<nucleoside B>” indicates a group in which one hydrogen atom is removed from the hydroxyl group at the position indicated by number B in the ribose part of nucleoside B. Specific abbreviations for groups used in the present disclosure mean the following structures, and for example, the notation “6rA-idA” indicates a structure in which the 3′-end of 6rA and the wavy part of the group represented by idA below form a phosphodiester bond with a monophosphorylated nucleic acid.

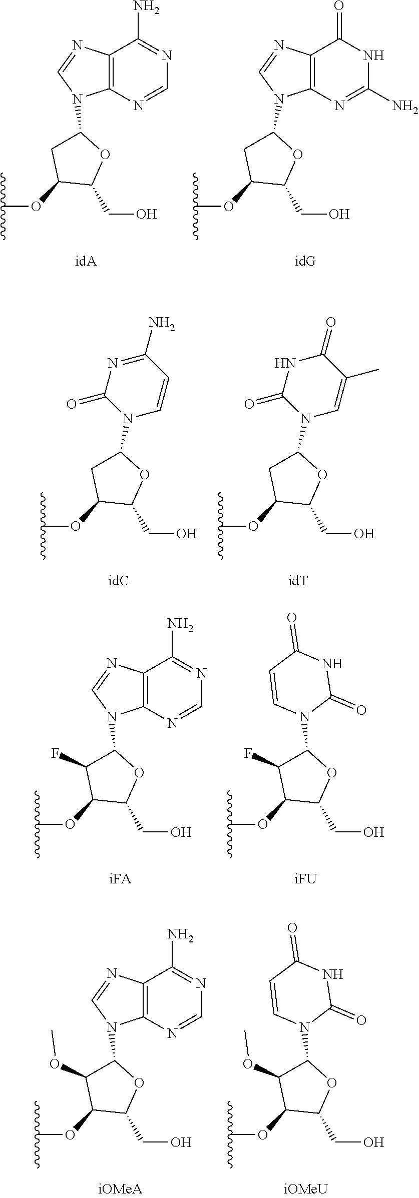

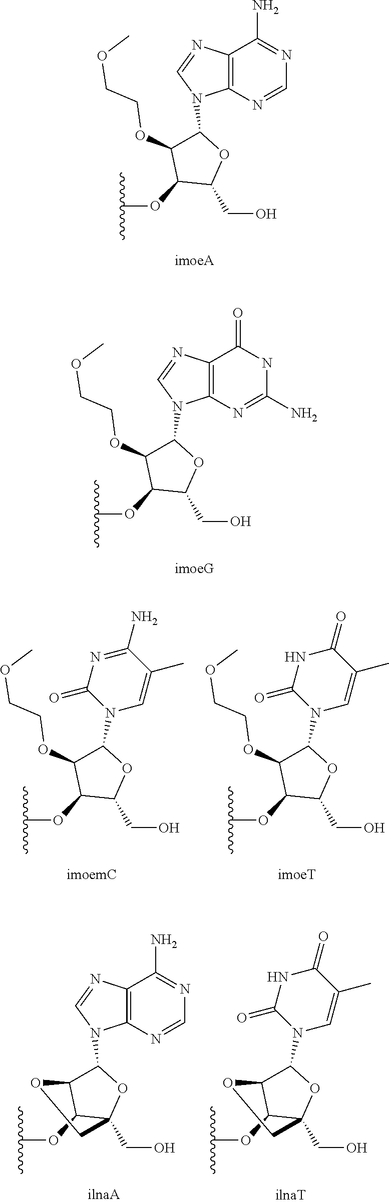

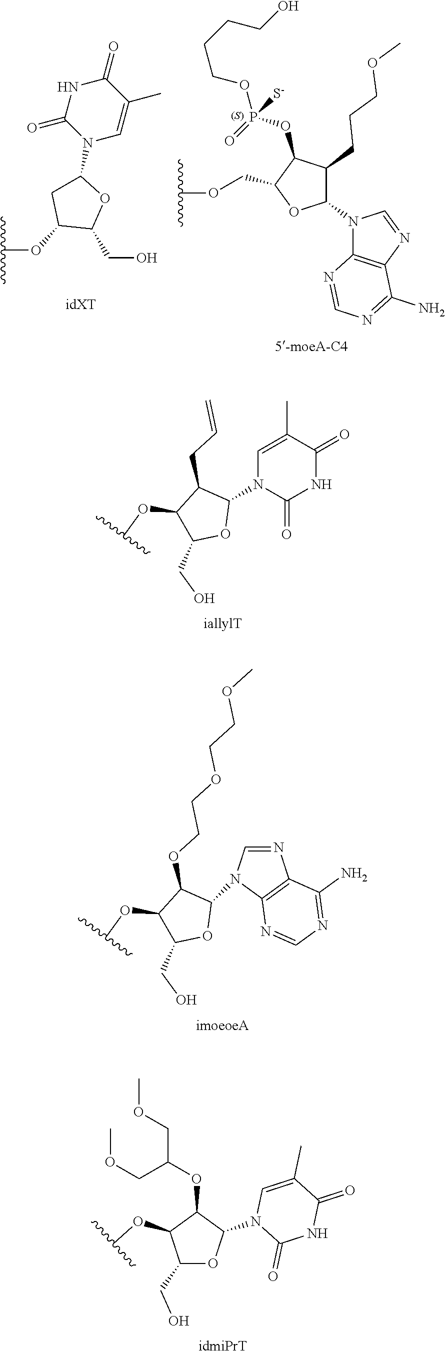

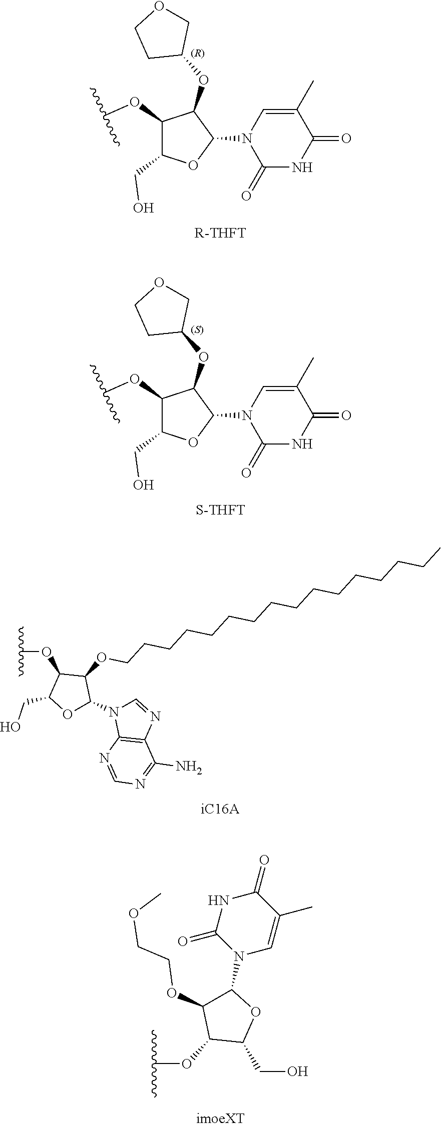

In a preferred embodiment of the present disclosure, Y in the above formula (I) is represented by the following formula (VI):

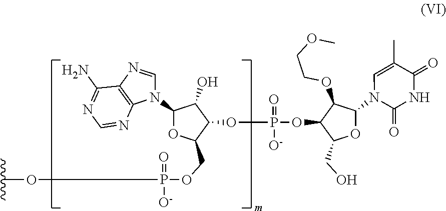

In formula (VI), the wavy line represents a bond to X, and m may represent an integer of 1 to 20. In one embodiment, m may represent an integer of 1 to 15, 1 to 10, 1 to 9, 1 to 8, 1 to 7, 1 to 6, 1 to 5, 1 to 4, 1 to 3, or 1 to 2. In a preferred embodiment, m may represent an integer of 1 to 6, and in a more preferred embodiment, m may represent an integer of 1 to 3. When Y in the above formula (I) is an oligonucleotide represented by the above formula (VI), the stability in vivo of the polynucleotide represented by X comprised in the polynucleotide represented by the above formula (I) is preferably improved, and its purity becomes high. Furthermore, when Y in the above formula (I) is an oligonucleotide represented by the above formula (VI), even if the nucleotide length of the oligonucleotide having a specific structure introduced to the 3′-end of the polynucleotide represented by the above formula (I) is extremely short, the stability in vivo is improved, and for example, even if m is 1, 2, or 3, the stability in vivo is suitably improved. As a result, for example, since the nucleotide length of the building block used for the introduction can be short, liquid-phase synthesis of the building block becomes possible, thereby enabling the inexpensive and highly efficient industrial production of the polynucleotide represented by the above formula (I).

A producing method of the polynucleotide according to the first aspect of the present disclosure described above is not particularly limited and can be produced by a method commonly used by those skilled in the art. The polynucleotide according to one embodiment of the first aspect of the present disclosure may be produced according to the producing method of the third aspect of the present disclosure described later. When the polynucleotide according to one embodiment of the first aspect of the present disclosure is produced according to the producing method of the third aspect of the present disclosure described later, its purity becomes high.

A second aspect of the present disclosure is a composition comprising the polynucleotide according to one embodiment of the first aspect of the present disclosure. The composition in this aspect means a composition comprising the polynucleotide according to one embodiment of the first aspect of the present disclosure, reagents added for production, and production intermediates, which may be generated or remain in a production of the polynucleotide according to one embodiment of the first aspect of the present disclosure.

A composition according to one embodiment may comprise the polynucleotide according to one embodiment of the first aspect of the present disclosure and a polynucleotide represented by the following formula (VII):

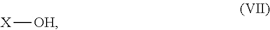

wherein, in formula (VII), X is the same as X in formula (I), and OH represents a hydroxyl group bonded to the 3′-carbon of the nucleoside present at the 3′-end of X. In this case, the content of the polynucleotide represented by the above formula (VII) may be suppressed in the composition according to one embodiment.

In the composition according to one embodiment, a content of the polynucleotide according to one embodiment of the first aspect of the present disclosure may be 50 mass % or more, 70 mass % or more, 80 mass % or more, 90 mass % or more, 95 mass % or more, 97 mass % or more, 98 mass % or more, or 99 mass % or more, and may be less than 100 mass %, 99 mass % or less, 98 mass % or less, 97 mass % or less, 96 mass % or less, 95 mass % or less, or 90 mass % or less, and these upper limits and these lower limits can be freely combined.

A third aspect of the present disclosure is a producing method of the polynucleotide according to one embodiment of the first aspect of the present disclosure. The producing method according to one embodiment of the third aspect of the present disclosure comprises contacting a polynucleotide represented by the following formula (VII):

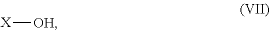

wherein, in formula (VII), X is the same as X in formula (I), and OH represents a hydroxyl group bonded to the 3′-carbon of a nucleoside present at the 3′-end of X, with an oligonucleotide represented by the following formula (VIII-A):

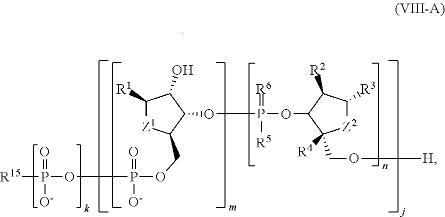

the following formula (IX):

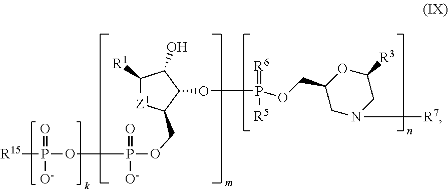

the following formula (X):

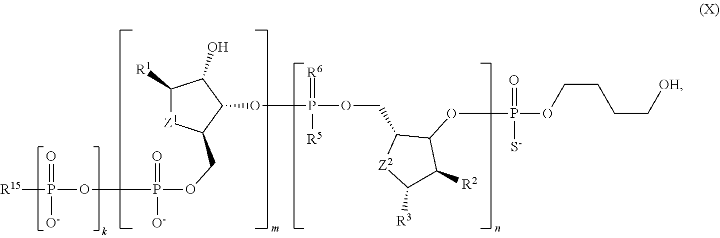

or the following formula (XI):

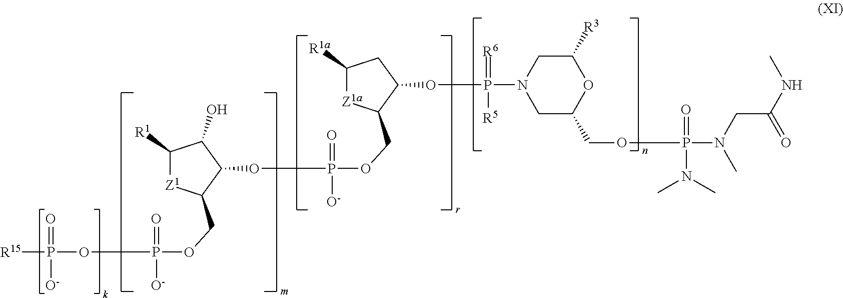

in the presence of RNA ligase or poly(A) polymerase to obtain the polynucleotide represented by formula (I) (contact step). In a preferred embodiment of the third aspect of the present disclosure, j in the above formula (VIII-A) may be 1. In other words, the producing method according to a preferred embodiment of the third aspect of the present disclosure comprises contacting a polynucleotide represented by the following formula (VII):

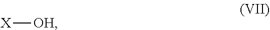

wherein, in formula (VII), X is the same as X in formula (I), and OH represents a hydroxyl group bonded to the 3′-carbon of a nucleoside present at the 3′-end of X, with an oligonucleotide represented by the following formula (VIII):

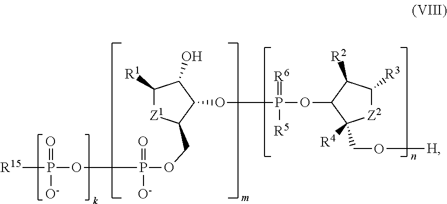

the following formula (IX):

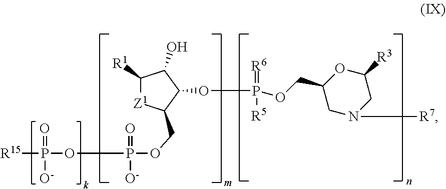

the following formula (X):

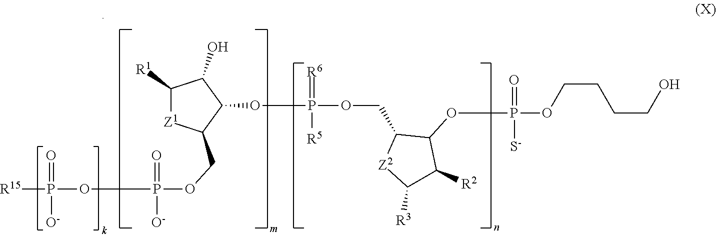

or the following formula (XI):

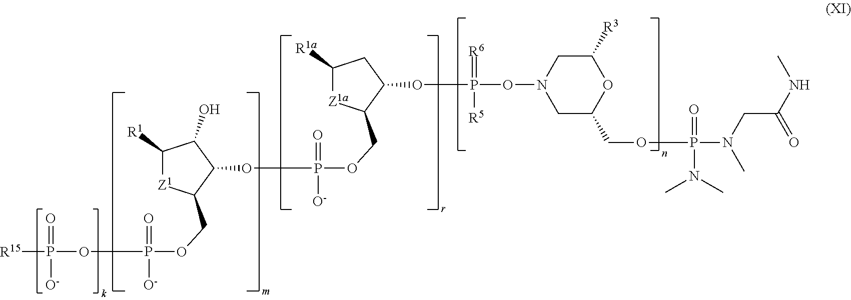

in the presence of RNA ligase or poly(A) polymerase to obtain the polynucleotide represented by formula (I), (contact step). One embodiment of the third aspect of the present disclosure is a producing method of the polynucleotide represented by the above formula (I), wherein Y in the above formula (I) is represented by the above formula (II-A), and comprises contacting a polynucleotide represented by the above formula (VII) with an oligonucleotide represented by the above formula (VIII-A) in the presence of RNA ligase or poly(A) polymerase to obtain the polynucleotide represented by the above formula (I). One embodiment of the third aspect of the present disclosure is a producing method of the polynucleotide represented by the above formula (I), wherein Y in the above formula (I) is represented by the above formula (II), and comprises contacting a polynucleotide represented by the above formula (VII) with an oligonucleotide represented by the above formula (VIII) in the presence of RNA ligase or poly(A) polymerase to obtain the polynucleotide represented by the above formula (I). One embodiment of the third aspect of the present disclosure is a producing method of the polynucleotide represented by the above formula (I), wherein Y in the above formula (I) is represented by the above formula (III), and comprises contacting a polynucleotide represented by the above formula (VII) with an oligonucleotide represented by the above formula (IX) in the presence of RNA ligase or poly(A) polymerase to obtain the polynucleotide represented by the above formula (I). One embodiment of the third aspect of the present disclosure is a producing method of the polynucleotide represented by the above formula (I), wherein Y in the above formula (I) is represented by the above formula (IV), and comprises contacting a polynucleotide represented by the above formula (VII) with an oligonucleotide represented by the above formula (X) in the presence of RNA ligase or poly(A) polymerase to obtain the polynucleotide represented by the above formula (I). One embodiment of the third aspect of the present disclosure is a producing method of the polynucleotide represented by the above formula (I), wherein Y in the above formula (I) is represented by the above formula (V), and comprises contacting a polynucleotide represented by the above formula (VII) with an oligonucleotide represented by the above formula (XI) in the presence of RNA ligase or poly(A) polymerase to obtain the polynucleotide represented by the above formula (I). In these cases, R¹ to R⁷, R^{1a}, Z¹, Z^{1a}, Z², j, m, n, and r in the above formulas (VIII), (VIII-A), (IX), (X), and (XI) are the same as those in formulas (II), (II-A), (III), (IV), or (V), and k is an integer of 0 to 2. The polynucleotide represented by the above formula (VII) may be a polynucleotide prepared by a method commonly used by those skilled in the art.

In formulas (VIII), (VIII-A), (IX), (X), and (XI), R¹⁵ represents O⁻ or a group obtained by removing one hydrogen atom from a hydroxyl group of the hydroxymethyl group at the 5′ p′-position of adenosine. Here, the group obtained by removing one hydrogen atom from a hydroxyl group of the hydroxymethyl group at the 5′-position of adenosine means a group represented by the following formula. In one embodiment, R¹⁵ may be O⁻.

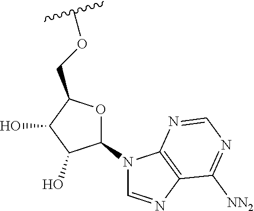

A producing method according to one embodiment of the third aspect of the present disclosure comprises contacting a polynucleotide represented by the following formula (VII):

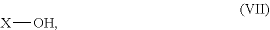

wherein, in formula (VII), X is the same as X in formula (I), and OH represents a hydroxyl group bonded to the 3′-carbon of a nucleoside present at the 3′-end of X, with an oligonucleotide represented by the following formula (VIII′-A):

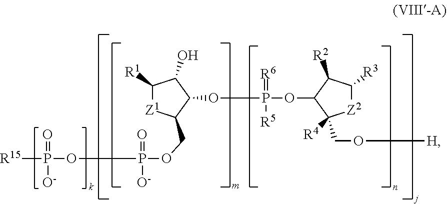

wherein, in formula (VIII′-A), R¹ to R⁶, Z¹, Z², j, m, and n are the same as those in formula (II′-A), and k represents an integer of 0 to 2, in the presence of RNA ligase or poly(A) polymerase to obtain the polynucleotide represented by the above formula (I) (contact step), wherein R¹⁵ represents O⁻ or a group obtained by removing one hydrogen atom from a hydroxyl group of the hydroxymethyl group at the 5′-position of adenosine, and in one embodiment, R¹⁵ may be O⁻. A producing method according to a preferred embodiment of the third aspect of the present disclosure comprises contacting a polynucleotide represented by the following formula (VII):

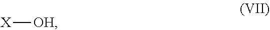

wherein, in formula (VII), X is the same as X in formula (I), and OH represents a hydroxyl group bonded to the 3′-carbon of a nucleoside present at the 3′-end of X, with an oligonucleotide represented by the following formula (VIII′):

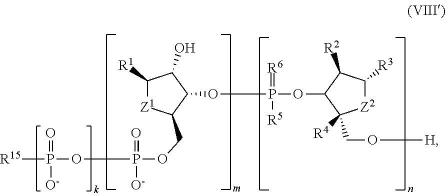

wherein, in formula (VIII′), R¹ to R⁶, Z¹, Z², m, and n are the same as those in formula (II′), and k represents an integer of 0 to 2, in the presence of RNA ligase or poly(A) polymerase to obtain the polynucleotide represented by the above formula (I) (contact step), wherein R¹⁵ represents O⁻ or a group obtained by removing one hydrogen atom from a hydroxyl group of the hydroxymethyl group at the 5′-position of adenosine, and in one embodiment, R¹⁵ may be O⁻. A producing method according to a more preferred embodiment of the third aspect of the present disclosure comprises contacting a polynucleotide represented by the following formula (VII):

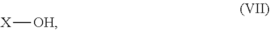

wherein, in formula (VII), X is the same as X in formula (I), and OH represents a hydroxyl group bonded to the 3′-carbon of a nucleoside present at the 3′-end of X, with an oligonucleotide represented by the following formula (XII):

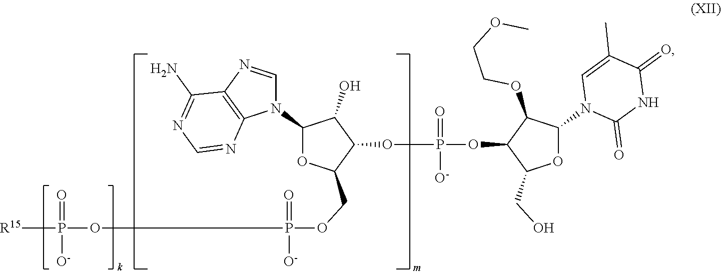

wherein, in formula (XII), m is the same as in formula (VI), and k represents an integer of 0 to 2, in the presence of RNA ligase or poly(A) polymerase to obtain the polynucleotide represented by the above formula (I) (contact step).

A producing method according to one embodiment of the third aspect of the present disclosure comprises contacting a polynucleotide represented by the following formula (VII):

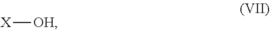

wherein, in formula (VII), X is the same as X in formula (I), and OH represents a hydroxyl group bonded to the 3′-carbon of a nucleoside present at the 3′-end of X, with an oligonucleotide represented by the following formula (VIII′-AX):

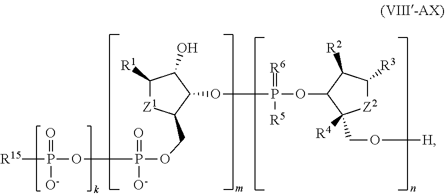

wherein, in formula (VIII′-AX), R¹ to R⁶, Z¹, Z², j, m, and n are the same as those in formula (II′-AX), and k represents an integer of 0 to 2, in the presence of RNA ligase or poly(A) polymerase to obtain the polynucleotide represented by the above formula (I) (contact step), wherein R¹⁵ represents O⁻ or a group obtained by removing one hydrogen atom from a hydroxyl group of the hydroxymethyl group at the 5′-position of adenosine, and in one embodiment, R¹⁵ may be O⁻. A producing method according to a preferred embodiment of the third aspect of the present disclosure comprises contacting a polynucleotide represented by the following formula (VII):

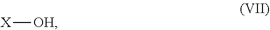

wherein, in formula (VII), X is the same as X in formula (I), and OH represents a hydroxyl group bonded to the 3′-carbon of a nucleoside present at the 3′-end of X, with an oligonucleotide represented by the following formula (VIII′-X):

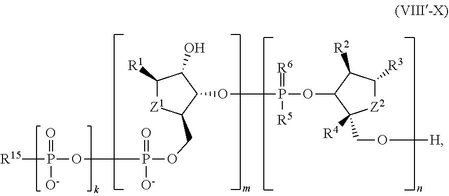

wherein, in formula (VIII′-X), R¹ to R⁶, Z¹, Z², m, and n are the same as those in formula (II′-X), and k represents an integer of 0 to 2, in the presence of RNA ligase or poly(A) polymerase to obtain the polynucleotide represented by the above formula (I) (contact step), wherein R¹⁵ represents O⁻ or a group obtained by removing one hydrogen atom from a hydroxyl group of the hydroxymethyl group at the 5′-position of adenosine, and in one embodiment, R¹⁵ may be O⁻.

In the producing method according to one embodiment, the RNA ligase or poly(A) polymerase may be RNA ligase. RNA ligase is an enzyme that catalyzes the formation of a phosphodiester bond between the 3′-end of one ribonucleotide and the 5′-end of another ribonucleotide. Examples of RNA ligase comprise T4 RNA ligase 1, T4 RNA ligase 2, and T4 DNA ligase. The T4 RNA ligase 2 may be, for example, a mutant T4 RNA ligase 2, such as T4 RNA Ligase 2, truncated, T4 RNA Ligase 2, truncated KQ, and T4 RNA Ligase 2, truncated K227Q (New England Biolabs, Inc.). When the producing method according to one embodiment uses RNA ligase, in formulas (VIII), (VIII′), (VIII′-X), (IX), (X), (XI), or (XII), for example, k may be 0 and R¹⁵ may be O⁻, or for example, k may be 1 and R¹⁵ may be a group obtained by removing one hydrogen atom from a hydroxyl group of the hydroxymethyl group at the 5′-position of adenosine.

In the producing method according to one embodiment, the RNA ligase or poly(A) polymerase may be poly(A) polymerase. Poly(A) polymerase is an enzyme that catalyzes the introduction of a poly(A) tail at the 3′-end of mRNA. When the producing method according to one embodiment uses poly(A) polymerase, in formulas (VIII), (VIII′), (VIII′-X), (VIII-A), (VIII′-A), (VIII′-AX), (IX), (X), (XI), or (XII), for example, k may be 2 and R¹⁵ may be O⁻.

In the contact step, the conditions for contacting the polynucleotide represented by the above formula (VII) with the oligonucleotide represented by formulas (VIII), (VIII′), (VIII′-X), (VIII-A), (VIII-A), (VIII′-AX), (IX), (X), (XI), or (XII) in the presence of RNA ligase or poly(A) polymerase are not particularly limited as long as the polynucleotide represented by the above formula (I) can be obtained, and the concentration relationships of each component, other additives and their concentrations, solvent, temperature, and reaction time can be those commonly used by those skilled in the art. Examples of the additives comprise ATP, divalent cations (Mg²⁺, Mn²⁺, etc.), reducing agents (DTT, etc.), RNase inhibitors, polyethylene glycol, pH adjusters, and buffers.

A fourth aspect of the present disclosure provides a lipid complex comprising: (I) the polynucleotide according to one embodiment of the first aspect of the present disclosure, (II) a cationic lipid, and (III) at least one lipid selected from the group consisting of neutral lipid, polyethylene glycol-modified lipid (PEG lipid), and sterol. Examples of the lipid complex herein comprise, but are not limited to, LNPs (lipid nanoparticles).

Examples of the form of a complex according to one embodiment of the fourth aspect of the present disclosure comprise a complex of the polynucleotide according to one embodiment of the first aspect of the present disclosure and a membrane consisting of a lipid monolayer (single molecule) (reverse micelle); a complex of the polynucleotide according to one embodiment of the first aspect of the present disclosure and a liposome; and a complex of the polynucleotide according to one embodiment of the first aspect of the present disclosure and a micelle. In a lipid complex according to an embodiment of the fourth aspect of the present invention, the polynucleotide according to one embodiment of the first aspect of the present disclosure is encapsulated in a fine particle comprising a lipid containing a cationic lipid.

The lipid complex according to one embodiment of the fourth aspect of the present disclosure contains the polynucleotide according to one embodiment of the first aspect of the present disclosure in a content of, for example, 0.01 to 50% by weight, 0.1 to 30% by weight, or 1 to 10% by weight to the total weight of the lipid complex.

Cationic lipid is an amphiphilic molecule comprising a lipophilic region including one or more hydrocarbon groups and a hydrophilic region including a polar group to be protonated at specific pH. Examples of the cationic lipid according to an embodiment comprise, but are not particularly limited to, cationic lipids described in International Publication Nos. WO 2015/105131, WO 2016/104580, and WO 2017/222016, and alternatively a cationic lipid with improved biodegradability described in International Publication No. WO 2016/104580 or WO 2017/222016 can be used. Examples of the cationic lipid according to an embodiment comprise 1-oxo-1-(undecan-5-yloxy)nonadecan-10-yl-1-methylpiperidine-4-carboxylate, 1-((2-butyloctyl)oxy)-1-oxononadecan-10-yl-1-methylpiperidine-4-carboxylate, 1-oxo-1-(undecan-5-yloxy)heptadecan-8-yl-1-methylpiperidine-4-carboxylate, 21-oxo-21-(undecan-5-yloxy)heneicosan-10-yl-1-methylpiperidine4-carboxylate, 21-(octan-3-yloxy)-21-oxoheneicosan-10-yl-1-methylpiperidine-4-carboxylate, 1-((2-butyloctyl)oxy)-1-oxoicosan-10-yl-1-methylpiperidine-4-carboxylate, (Z)-1-((2-butylnon-3-en-1-yl)oxy)-1-oxoicosan-10-yl-1-methylpiperidine-4-carboxylate, 1-oxo-1-((3-pentyloctyl)oxy)icosan-10-yl-1-methylpiperidine-4-carboxylate, 1-((3,4-dipropylheptyl)oxy)-1-oxoicosan-10-yl-1-methylpiperidine-4-carboxylate, 1-((6-(butyldisulfanyl)-3-(3-(butyldisulfanyl)propyl)hexyl)oxy)-1-oxoicosan-10-yl-1-methylpiperidine-4-carboxylate, 2-butyloctyl-10-((4-(dimethylamino)butanoyl)oxy)icosanoate, 2-{9-[(2-butyloctyl)oxy]-9-oxononyl}dodecyl-1-methylpiperidine-4-carboxylate, 2-{9-oxo-9-[(3-pentyloctyl)oxy]nonyl}dodecyl 1-methylpiperidine-4-carboxylate, 2-nonyl-11-oxo-11-[(3-pentyloctyl)oxy]undecyl-1-methylpiperidine-4-carboxylate, bis(3-pentyloctyl) 9-{[(1-methylpiperidine-4-carbonyl)oxy]methyl}heptadecanedioate, and di[(Z)-2-nonen-1-yl]-9-{[(1-methylpiperidine-4-carbonyl)oxy]methyl}heptadecanedioate. In an embodiment, the cationic lipid is 2-{9-oxo-9-[(3-pentyloctyl)oxy]nonyl}dodecyl-1-methylpiperidine-4-carboxylate.

The lipid complex according to one embodiment of the fourth aspect of the present disclosure contains the above-described cationic lipid in a content of, for example, 10 to 100 mol %, 20 to 90 mol %, or 40 to 70 mol % based on the total lipids contained in the lipid complex. One cationic lipid can be used singly, and mixture of two or more cationic lipids can also be used.

The lipid complex according to one embodiment of the fourth aspect of the present disclosure comprises (I) the above-described cationic lipid and (II) at least one lipid selected from the group consisting of neutral lipid, polyethylene glycol-modified lipid, and sterol, as a lipid component. The lipid complex according to one embodiment of the fourth aspect of the present disclosure contains the lipid component in a content of, for example, 50 to 99.99% by weight, 70 to 99.9% by weight, or 90 to 99% by weight to the total weight of the lipid complex.

The term “neutral lipid” refers to a lipid present either as a non-charged form or as a neutral zwitterion at physiological pH. Examples of the neutral lipid according to an embodiment comprise dioleoylphosphatidylethanolamine (DOPE), palmitoyloleoylphosphatidylcholine (POPC), egg phosphatidylcholine (EPC), dimyristoylphosphatidylcholine (DMPC), dipalmitoylphosphatidylcholine (DPPC), distearoylphosphatidylcholine (DSPC), diarachidoylphosphatidylcholine (DAPC), dibehenoylphosphatidylcholine (DBPC), dilignoceroylphosphatidylcholine (DLPC), dioleoylphosphatidylcholine (DOPC), sphingomyelin, ceramide, dioleoylphosphatidylglycerol (DOPG), dipalmitoylphosphatidylglycerol (DPPG), palmitoyloleoylphosphatidylethanolamine (POPE), and dioleoyl-phosphatidylethanolamine 4-(N-maleimidomethyl)-cyclohexane-1-carboxylate (DOPE-mal). In an embodiment, the neutral lipid is distearoylphosphatidylcholine (DSPC). One neutral lipid can be used singly, and mixture of two or more neutral lipids can also be used.

The lipid complex according to one embodiment of the fourth aspect of the present disclosure may contain the neutral lipid in a content of, for example, 0 to 50 mol %, 0 to 40 mol %, 0 to 30 mol %, or 0 to 20 mol % based on the total lipids contained in the lipid complex.

Examples of the polyethylene glycol-modified lipid (PEG lipid) according to one embodiment of the fourth aspect of the present disclosure comprise PEG2000-DMG (PEG2000-dimyristyl glycerol), MPEG2000-DMG (MPEG2000-dimyristyl glycerol), PEG2000-DPG (PEG2000-dipalmitoylglycerol), PEG2000-DSG (PEG2000-distearoylglycerol), PEG5000-DMG (PEG5000-dimyristyl glycerol), PEG5000-DPG (PEG5000-dipalmitoylglycerol), PEG5000-DSG (PEG5000-distearoylglycerol), PEG-CDMA (N-[(methoxypoly(ethylene glycol) 2000) carbamyl]-1,2-dimyristyloxylpropyl-3-amine), PEG-C-DOMG (R-3-[(ω-methoxy-poly(ethylene glycol)2000)carbamoyl)]-1,2-dimyristyloxylpropyl-3-amine), polyethylene glycol (PEG)-diacylglycerol (DAG), PEG-dialkyloxypropyl (DAA), PEG-phospholipid, and PEG-ceramide (Cer). Examples of PEG-dialkyloxypropyl comprise PEG-dilauryloxypropyl, PEG-dimyristyloxypropyl, PEG-dipalmityloxypropyl, and PEG-distearyloxypropyl. In an embodiment, the polyethylene glycol-modified lipid is MPEG2000-DMG (MPEG2000-dimyristyl glycerol). One polyethylene glycol-modified lipid can be used singly, and mixture of two or more polyethylene glycol-modified lipids can also be used.

The lipid complex according to one embodiment of the fourth aspect of the present disclosure may contain the polyethylene glycol-modified lipid in a content of, for example, 0 to 30 mol %, 0 to 20 mol %, 0 to 10 mol %, or 0.5 to 2 mol % based on the total lipids contained in the lipid complex.

Sterol is an alcohol having a steroid backbone. In some embodiments, the sterol comprises cholesterol, dihydrocholesterol, lanosterol, β-sitosterol, campesterol, stigmasterol, brassicasterol, ergosterol, fucosterol, and 3β-[N—(N′,N′-dimethylaminoethyl)carbamoyl]cholesterol (DC-Chol). In an embodiment, the sterol is cholesterol. One sterol can be used singly, and mixture of two or more sterols can also be used.

The lipid complex according to one embodiment of the fourth aspect of the present disclosure may contain the sterol in a content of, for example, 0 to 90 mol %, 10 to 80 mol %, or 20 to 40 mol % based on the total lipids contained in the lipid complex.

Combination of lipid components in the lipid complex according to one embodiment of the fourth aspect of the present disclosure is not particularly limited, and examples thereof comprise combination of the above-described cationic lipid, neutral lipid, and sterol, and combination of the above-described cationic lipid, neutral lipid, polyethylene glycol-modified lipid, and sterol.

The lipid complex according to one embodiment of the fourth aspect of the present disclosure comprises lipid components of cationic lipid/neutral lipid/polyethylene glycol-modified lipid/sterol, and the mole ratio of the lipids may be, for example, 10 to 99/0 to 50/0 to 10/0 to 50, or 40 to 70/0 to 20/0.5 to 2/20 to 40.

The “average particle size” of the lipid complex particle according to one embodiment of the fourth aspect of the present disclosure refers to the Z-average particle size. The average particle size (Z-average) of a lipid complex according to one embodiment of the fourth aspect of the present disclosure may be, for example, 10 to 1000 nm, 30 to 500 nm, or 30 to 200 nm as measured by using a particle size analyzer (Malvern Panalytical Ltd., Zetasizer Nano ZS), though the average particle size is not particularly limited thereto.

A fifth aspect of the present disclosure is an oligonucleotide represented by the above formulas (VIII), (VIII′), (VIII′-X), (VIII-A), (VIII′-A), (VIII′-AX), (IX), (X), (XI), or (XII). R¹ to R⁷, R^{1a}, R¹⁵, Z¹, Z^{1a}, Z², j, m, n, and r in these formulas are the same as those described in the third aspect of the present disclosure, and k is an integer of 0 to 2, and in one embodiment, k is 0 or 2. These oligonucleotides can be used, for example, as a building block in the producing method according to the third aspect of the present disclosure.

A sixth aspect of the present disclosure is a method of improving the stability of a polynucleotide in vivo, comprising introducing a group represented by the above formulas (II), (II-A), (III), (IV), or (V) to the 3′-end of the polynucleotide, wherein, in the formulas, the wavy line represents a bond to the 3′-end of the polyribonucleotide, and R¹ to R⁷, R^{1a}, R¹⁵, Z¹, Z^{1a}, Z², j, m, n, and r are the same as those described for formulas (II), (II-A), (III), (IV), or (V) in the first aspect of the present disclosure.

A preferred embodiment of the sixth aspect of the present disclosure is a method of improving the stability of a polynucleotide in vivo, comprising introducing a group represented by the above formula (II-A) to the 3′-end of the polynucleotide, wherein, in the formula, the wavy line represents a bond to the 3′-end of the polyribonucleotide, and R¹ to R⁶, Z¹, Z², j, m, and n are the same as those described for formula (II-A) in the first aspect of the present disclosure.
A preferred embodiment of the sixth aspect of the present disclosure is a method of improving the stability of a polynucleotide in vivo, comprising introducing a group represented by the above formula (II) to the 3′-end of the polynucleotide, wherein in the formula, the wavy line represents a bond to the 3′-end of the polyribonucleotide, and R¹ to R⁶, Z¹, Z², m, and n are the same as those described for formula (II) in the first aspect of the present disclosure.
A more preferred embodiment of the sixth aspect of the present disclosure is a method of improving the stability of a polynucleotide in vivo, comprising introducing a group represented by the above formula (VI) to the 3′-end of the polynucleotide, wherein, in the formula, the wavy line represents a bond to the 3′-end of the polyribonucleotide, and m is the same as described for formula (VI) in the first aspect of the present disclosure.
The introduction of the above group to the 3′-end of the polynucleotide can be carried out by methods commonly used by those skilled in the art, for example, by a method similar to the contact step in the producing method according to one embodiment of the third aspect of the present disclosure.

The polynucleotide according to the first aspect of the present disclosure, and the polynucleotide with the above group introduced at the 3′-end according to the method for improving stability in vivo of the sixth aspect of the present disclosure, have high stability in vivo. In these cases, high stability means that, for example, a total amount of the polynucleotide containing the group represented by X in the culture supernatant or blood 24 hours, 48 hours, 72 hours, 96 hours, or 168 hours after administration to a subject (e.g., human, mouse, or rat) is 2 times, 3 times, 5 times, 7 times, 10 times, 30 times, 100 times, 250 times, or 500 times or more compared to a total amount when the same amount of the polynucleotide represented by the following formula (VII):

X—OH (VII)

is administered, wherein in formula (VII), X is the same as X in formula (I), and OH represents a hydroxyl group bonded to the 3′-carbon of the nucleoside present at the 3′-end of X. The amount of the polynucleotide may be measured by a method such as quantitative PCR (q-PCR) or next-generation sequencing.

A seventh aspect of the present disclosure is a method for improving an expression level of a protein encoded by a coding region in a polyribonucleotide, comprising introducing a group represented by the above formula (II), (II-A), (III), (IV), or (V) to the 3′-end of the polyribonucleotide, wherein the wavy line represents a bond to the 3′-end of the polyribonucleotide, and R¹ to R⁷, R^{1a}, Z¹, Z^{1a}, Z², m, n, and r are the same as those described for formulas (II), (II-A), (III), (IV), and (V) in the first aspect of the present disclosure. A preferred embodiment of the seventh aspect of the present disclosure is a method for improving the expression level of a protein encoded by a coding region in a polyribonucleotide, comprising introducing a group represented by the above formula (II-A) in the first aspect of the present disclosure to the 3′-end of the polyribonucleotide, wherein the wavy line represents a bond to the 3′-end of the polyribonucleotide, and R¹ to R⁶, Z¹, Z², j, m, and n are the same as those described for formula (II-A). A preferred embodiment of the seventh aspect of the present disclosure is a method for improving the expression level of a protein encoded by a coding region in a polyribonucleotide, comprising introducing a group represented by the above formula (II) in the first aspect of the present disclosure to the 3′-end of the polyribonucleotide, wherein the wavy line represents a bond to the 3′-end of the polyribonucleotide, and R¹ to R⁶, Z¹, Z², m, and n are the same as those described for formula (II). A more preferred embodiment of the seventh aspect of the present disclosure is a method for improving the expression level of a protein encoded by a coding region in a polyribonucleotide, comprising introducing a group represented by the above formula (VI) to the 3′-end of the polyribonucleotide, wherein the wavy line represents a bond to the 3′-end of the polyribonucleotide, and m is the same as described for formula (VI) in the first aspect of the present disclosure. The introduction of the above group to the 3′-end of the polyribonucleotide can be carried out by a method commonly used by those skilled in the art, for example, by a method similar to the contact step in the producing method according to one embodiment of the third aspect of the present disclosure.

When the polyribonucleotide according to one embodiment of the first aspect of the present disclosure, the polyribonucleotide with the above group introduced at the 3′-end according to the method for improving stability in vivo of one embodiment of the sixth aspect of the present disclosure, or the polyribonucleotide with the above group introduced at the 3′-end according to the method for improving the expression level of a protein of one embodiment of the seventh aspect of the present disclosure are administered to a subject, the amount of the protein encoded by the coding region increases after a long period of time post-administration. In the sixth aspect of the present disclosure, “high stability of the polyribonucleotide in vivo” and in the seventh aspect, “high amount of the protein encoded by the coding region” mean that, for example, the amount (e.g., enzyme units per volume such as mU/mL, or molar concentration) of the protein encoded by the coding region in the culture supernatant or blood 36 hours, 48 hours, 72 hours, 96 hours, or 168 hours after administration to a subject (e.g., cell, human, mouse, or rat) is 2 times or more, 3 times or more, 4 times or more, 5 times or more, 6 times or more, 7 times or more, 8 times or more, 9 times or more, 10 times or more, 20 times or more, 30 times or more, 50 times or more, 100 times or more, 200 times or more, 300 times or more, 400 times or more, 500 times or more, 750 times or more, 1000 times or more, 1250 times or more, 1500 times or more, 1700 times or more, or 1900 times or more compared to the amount when the same amount of the polynucleotide represented by the following formula (VII):

X—OH (VII)

is administered, wherein, in formula (VII), X is the same as X in formula (I), and OH represents a hydroxyl group bonded to the 3′-carbon of the nucleoside present at the 3′-end of X.
An example of a high amount of the protein encoded by the coding region is that the amount of the protein encoded by the coding region in the culture supernatant 96 hours or 168 hours after administration to cells is 2 times or more, 3 times or more, 4 times or more, 5 times or more, 6 times or more, 7 times or more, 8 times or more, 9 times or more, or 10 times or more compared to the amount when the same amount of the polynucleotide represented by the above formula (VII) is administered, and in one embodiment, it may be 3 times or more compared to the same.
An example of a high amount of the protein encoded by the coding region is that the amount of the protein encoded by the coding region in the blood 48 hours, 72 hours, 96 hours, or 168 hours after administration to a mouse or human is 5 times or more, 7 times or more, 10 times or more, 20 times or more, 30 times or more, 100 times or more, 250 times or more, 500 times or more, 750 times or more, 1000 times or more, 1250 times or more, 1500 times or more, 1700 times or more, or 1900 times or more compared to the amount when the same amount of the polynucleotide represented by the above formula (VII) is administered, and in one embodiment, it may be 750 times or more compared to the same.
“High stability of the polyribonucleotide in vivo” in the sixth aspect of the present disclosure and “high amount of the protein encoded by the coding region” in the seventh aspect mean that, for example, the amount of the protein encoded by the coding region in the culture supernatant or blood 72 hours after administration to a subject (e.g., cell, human, mouse, or rat) is 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, or 30% or more of the amount of the protein 24 hours after administration.
An example of a high amount of the protein encoded by the coding region is that the amount of the protein encoded by the coding region in the blood 96 hours after administration to a mouse is 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, or 20% or more of the amount 24 hours after administration, and in one embodiment, it May 10% or more of the same.
An example of a high amount of the protein encoded by the coding region is that the signal intensity detected by a reporter assay (e.g., luminescence intensity generated by substrate addition) 24 hours, 48 hours, or 72 hours after introducing a polynucleotide (e.g., a polynucleotide consisting of the nucleotide sequence shown in SEQ ID NO: 1) encoding a reporter protein as X into cells (e.g., Hep3B cells) is 130% or more, 150% or more, 155% or more, 160% or more, 165% or more, 170% or more, 175% or more, 180% or more, 185% or more, 190% or more, 195% or more, or 200% or more of the signal intensity at the same time point when the same amount of the polynucleotide represented by the above formula (VII) is introduced. The detailed method of such an evaluation may follow the protocol performed in Example 2 described later.

An eighth aspect of the present disclosure is a kit comprising the polynucleotide according to one embodiment of the fourth aspect of the present disclosure. The kit according to the eighth aspect of the present disclosure may be a kit for improving the stability of the polynucleotide. The kit according to the eighth aspect of the present disclosure may be a kit for improving the expression level of a protein encoded by a coding region contained in the polyribonucleotide. The kit according to the eighth aspect of the present disclosure may further comprise RNA ligase or poly(A) polymerase. The kit according to the eighth aspect of the present disclosure may further comprise an instruction manual, which may be electronic, and the instruction manual may comprise descriptions corresponding to the producing method according to one embodiment of the third aspect of the present disclosure, the method for improving stability according to one embodiment of the sixth aspect of the present disclosure, or the method for improving the expression level according to one embodiment of the seventh aspect of the present disclosure.

### EXAMPLES

Hereinafter, the present disclosure will be described in more detail referring to examples, but the present disclosure is not limited to the following examples.

### [Example 1] Preparation of PolyA-modified Fluc-mRNA and PolyA-modified EPO-mRNA

### <Synthesis of Monomer>

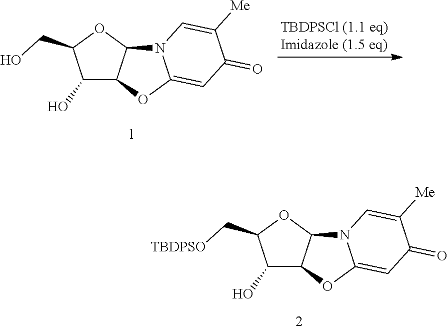

### (2R,3R,3aS,9aR)-2-(((tert-butyldiphenylsilyl)oxy)methyl)-3-hydroxy-7-methyl-2,3,3a,9a-tetrahydro-6H-furo[2′,3′:4,5]oxazolo[3,2-a]pyrimidin-6-one 2

To a 0° C. stirred solution of 2,2′-Anhydro-5-methyluridine 1 (CAS No 22423-26-3, 100.00 g, 0.42 mol) and Imidazole (42.51 g, 0.62 mol) in dry N,N-Dimethylformamide (500 mL) was added dropwise a solution of TBDPSCl (125.86 g, 0.46 mol) in dry dichloromethane (200 mL). After the addition, the mixture was warmed back to room temperature and stirred overnight. After the starting material was consumed, the mixture was concentrated to remove dichloromethane. Then the residue was carefully poured into cold water (2 L) with vigorous stirring. The precipitated solid was filtered and washed with water (300 mL), hexane (300 mL) and diethyl ether (300 mL) successively. The solid was collected and dried over vacuum to afford 2 (154.30 g, 77%) as white solid.

¹H NMR (400 MHZ, DMSO-d6) δ 7.79 (s, 1H), 7.56-7.49 (m, 4H), 7.47-7.37 (m, 6H), 6.31 (d, J=5.7 Hz, 1H), 5.99 (d, J=4.5 Hz, 1H), 5.23 (dd, J=5.8, 1.4 Hz, 1H), 4.44-4.39 (m, 1H), 4.18 (ddd, J=7.5, 4.6, 3.1 Hz, 1H), 3.57 (dd, J=11.3, 4.8 Hz, 1H), 3.41 (dd, J=11.4, 6.8 Hz, 1H), 1.76 (s, 3H), 0.91 (s, 9H).

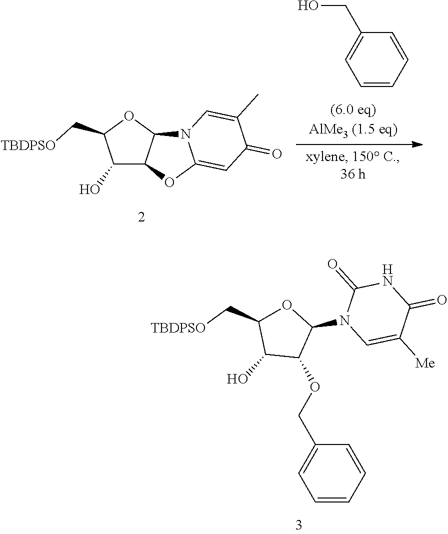

### 1-((2R,3R,4R,5R)-3-(benzyloxy)-5-(((tert-butyldiphenylsilyl)oxy)methyl)-4-hydroxytetrahydrofuran-2-yl)-5-methylpyrimidine-2,4(1H,3H)-dione 3

To a stirred solution of phenylmethanol (33.89 g, 0.31 mol) in xylene (20 mL) was added AlMe₃ (5.65 g, 78.35 mmol, 2 M in toluene) drop-wise allowing gentle reflux. After the addition, the mixture was stirred at 110° C. for 2 hours. Then the resulting mixture was cooled to RT, transferred to a round-bottomed flask containing a suspension of 2 (25.00 g, 52.23 mmol) in xylene (75 mL) and stirred at 150° C. for 36 h. After the starting material 2 was consumed, the mixture was poured into 15% potassium sodium tartrate solution (1 L) and stirred for 20 min. After filtration, the aqueous layer was extracted with DCM (500 mL×2). The combined organic phase was washed with water (500 mL) and brine (500 mL), dried over Na₂SO₄ and concentrated to dryness. The residue was purified by silica gel column (DCM/MeOH-50/1, v/v) to afford 3 (13.08 g, 43%) as a white solid.

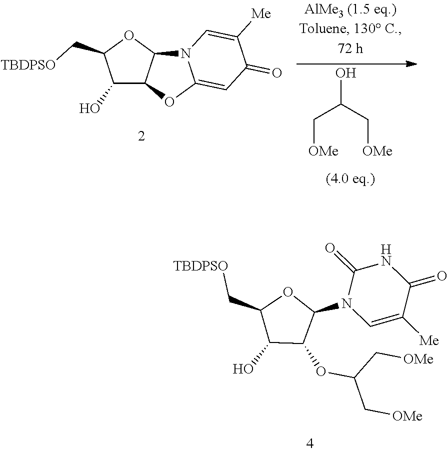

### 1-((2R,3R,4R,5R)-5-(((tert-butyldiphenylsilyl)oxy)methyl)-3-((1,3-dimethoxypropan-2-yl)oxy)-4-hydroxytetrahydrofuran-2-yl)-5-methylpyrimidine-2,4(1H,3H)-dione 4

To a stirred solution of 1,3-dimethoxypropan-2-ol (20.08 g, 0.17 mol) in toluene (20 mL) was added AlMe₃ (4.52 g, 62.68 mmol, 2 M in toluene) drop-wise allowing gentle reflux. After the addition, the mixture was stirred at 110° C. for 1 h. Then the resulting mixture was cooled to room temperature, transferred to a pressure flask containing a suspension of 2 (20.00 g, 41.79 mmol) in toluene (25 mL) and stirred at 130° C. for 72 hours. After the most 2 was consumed, the mixture was poured into 15% potassium sodium tartrate solution (1 L) and stirred for 20 min. After filtration, the aqueous layer was extracted with DCM (500 mL×2). The combined organic phase was washed with water (500 mL) and brine (500 mL), dried over Na₂SO₄, and concentrated to dryness. The residue was purified by a silica gel column (DCM/MeOH=50/1, v/v) to afford 4 (3.20 g, 13%) as white solid.

¹H NMR (400 MHZ, DMSO-d6) δ 11.38 (s, 1H), 7.67-7.62 (m, 4H), 7.50-7.41 (m, 6H), 7.38 (s, 1H), 5.90 (d, J=6.0 Hz, 1H), 4.79 (d, J=4.5 Hz, 1H), 4.26 (dt, J=12.6, 4.9 Hz, 2H), 3.96 (q, J=3.2 Hz, 1H), 3.88 (dd, J=11.6, 2.7 Hz, 1H), 3.81 (dd, J=11.6, 3.4 Hz, 1H), 3.77-3.71 (m, 1H), 3.44-3.38 (m, 2H), 3.37 (d, J=4.0 Hz, 1H), 3.28 (s, 3H), 3.27-3.22 (m, 1H), 3.14 (s, 3H), 1.42 (s, 3H), 1.04 (s, 9H).

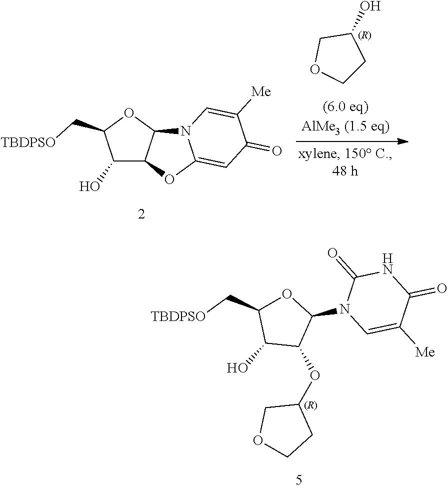

### 1-((2R,3R,4R,5R)-5-(((tert-butyldiphenylsilyl)oxy)methyl)-4-hydroxy-3-(((R)-tetrahydrofuran-3-yl)oxy)tetrahydrofuran-2-yl)-5-methylpyrimidine-2,4(1H,3H)-dione 5

To a stirred solution of (R)-tetrahydrofuran-3-ol (8.84 g, 0.1 mol) in xylenes (10 mL) was added AlMe₃ (1.81 g, 25.07 mmol, 2 M in toluene) drop-wise allowing gentle reflux. After the addition, the mixture was stirred at 110° C. for 2 hours. Then the resulting mixture was cooled to room temperature, transferred to a round-bottomed flask containing a suspension of 2 (8.00 g, 16.71 mmol) in xylene (40 mL) and stirred at 150° C. for 48 h. After the most 2 was consumed, the mixture was poured into 15% potassium sodium tartrate solution (500 mL) and stirred for 20 min. After filtration, the aqueous layer was extracted with DCM (200 mL×2). The combined organic phase was washed with water (200 mL) and brine (200 mL), dried over Na₂SO₄, and concentrated to dryness. The residue was purified by a silica gel column (DCM/MeOH=50/1, v/v) to afford 5 (3.00 g, 32%) as white solid.

¹H NMR (400 MHZ, DMSO-d6) δ 11.39 (s, 1H), 7.65 (td, J=8.2, 1.7 Hz, 4H), 7.49-7.40 (m, 7H), 5.88 (d, J=5.6 Hz, 1H), 5.18 (d, J=5.7 Hz, 1H), 4.32 (dq, J=6.7, 2.2 Hz, 1H), 4.22 (q, J=5.0 Hz, 1H), 4.04 (t, J=5.4 Hz, 1H), 3.99-3.92 (m, 2H), 3.83 (dd, J=11.4, 3.6 Hz, 1H), 3.71-3.63 (m, 4H), 1.94-1.85 (m, 1H), 1.77-1.69 (m, 1H), 1.43 (s, 3H), 1.04 (s, 9H).

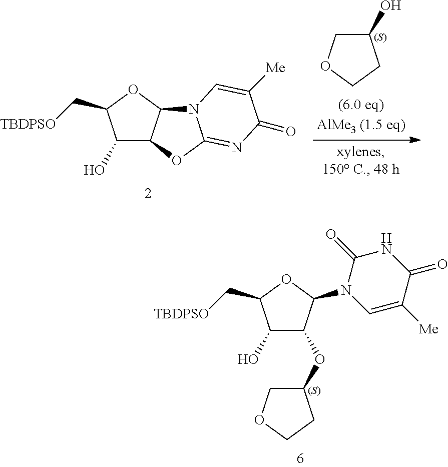

### 1-((2R,3R,4R,5R)-5-(((tert-butyldiphenylsilyl)oxy)methyl)-4-hydroxy-3-(((S)-tetrahydrofuran-3-yl)oxy)tetrahydrofuran-2-yl)-5-methylpyrimidine-2,4(1H,3H)-dione 6

To a stirred solution of(S)-tetrahydrofuran-3-ol (27.61 g, 0.31 mol) in xylenes (20 mL) was added AlMe₃ (5.65 g, 78.35 mmol, 2 M in toluene) drop-wise allowing gentle reflux. After the addition, the mixture was stirred at 110° C. for 2 hours. Then the resulting mixture was cooled to RT, transferred to a round-bottomed flask containing a suspension of 2 (25.00 g, 52.23 mmol) in xylenes (75 mL) and stirred at 150° C. for 48 h. After most 2 was consumed, the mixture was poured into 15% potassium sodium tartrate solution (1 L) and stirred for 20 min. After filtration, the aqueous layer was extracted with DCM (500 mL×2). The combined organic phase was washed with water (500 mL) and brine (500 mL), dried over Na₂SO₄, and concentrated to dryness. The residue was purified by a silica gel column (DCM/MeOH=50/1, v/v) to afford 6 (8.93 g, 30%) as white solid.

¹H NMR (400 MHZ, DMSO-d6) δ 11.38 (s, 1H), 7.67-7.62 (m, 4H), 7.50-7.40 (m, 7H), 5.87 (d, J=5.2 Hz, 1H), 5.19 (d, J=6.1 Hz, 1H), 4.32 (dq, J=6.1, 3.1, 2.6 Hz, 1H), 4.21 (q, J=5.2 Hz, 1H), 4.06-3.98 (m, 2H), 3.98-3.91 (m, 2H), 3.83 (dd, J=11.3, 3.6 Hz, 1H), 3.68-3.61 (m, 2H), 3.58 (dd, J=9.8, 1.9 Hz, 1H), 1.97-1.90 (m, 2H), 1.44 (s, 3H), 1.04 (s, 9H).

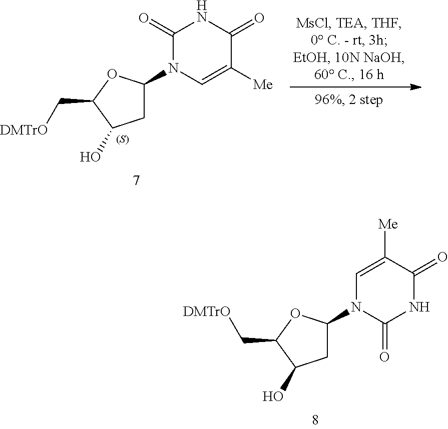

### 1-((2R,4R,5R)-5-((bis(4-methoxyphenyl) (phenyl) methoxy)methyl)-4-hydroxytetrahydrofuran-2-yl)-5-methylpyrimidine-2,4(1H,3H)-dione 8

Inversion of 3′-OH was performed by following a literature procedure (Duerr, E.-M.; McGouran, J. F. Molecules 2021, 26, 320). 1-((2R,4S,5R)-5-((bis(4-methoxyphenyl) (phenyl) methoxy)methyl)-4-hydroxytetrahydrofuran-2-yl)-5-methylpyrimidine-2,4(1H,3H)-dione 7 (5.00 g, 9.181 mmol) in Tetrahydrofuran (41.7 mL) was added triethylamine (3.20 mL, 22.952 mmol). Then, Methanesulfonyl chloride (1.06 mL, 13.77 mmol) was added dropwise at 0° C. and stirred at room temperature. Upon the completion of the reaction, Ethanol (20 mL, 0.343 mmol) was added followed by solution of 10 N NaOH (50 mL). The mixture was heated to 60° C. overnight. It was diluted with EtOAc, and pH was adjusted to 7 by 4 N HCl. After extraction with EtOAc and concentration, the crude was purified by a column chromatography (100 g Sfar Biotage column, DCM/MeOH 1 to 5%) to provide 8 (4.8 g, 8.81 mmol, 96% yield).

¹H NMR (400 MHz, DMSO-d₆) δ 1.62-1.66 (m, 3H), 1.79-1.93 (m, 1H), 2.52-2.58 (m, 1H), 3.18 (dd, J=10.32, 2.81 Hz, 1H), 3.38 (dd, J=10.26, 8.13 Hz, 1H), 3.73 (d, J=0.63 Hz, 6H), 4.03-4.13 (m, 1H), 4.14-4.25 (m, 1H), 5.19 (d, J=3.50 Hz, 1H), 6.10 (dd, J=8.13, 2.13 Hz, 1H), 6.84-6.91 (m, 4H), 7.19-7.32 (m, 7H), 7.43 (d, J=7.66 Hz, 2H), 7.59 (d, J=1.13 Hz, 1H), 11.27 (s, 1H).

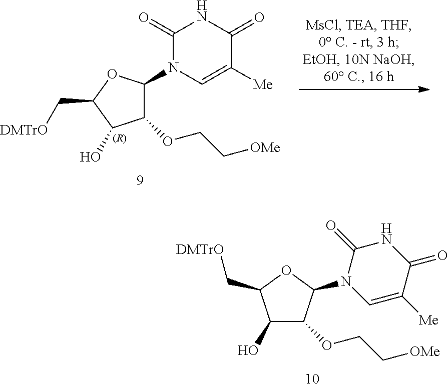

### 1-((2R,3R,4S,5R)-5-((bis(4-methoxyphenyl) (phenyl) methoxy)methyl)-4-hydroxy-3-(2-methoxyethoxy)tetrahydrofuran-2-yl)-5-methylpyrimidine-2,4(1H,3H)-dione 10

1-((2R,3R,4R,5R)-5-((bis(4-methoxyphenyl) (phenyl) methoxy)methyl)-4-hydroxy-3-(2-methoxyethoxy)tetrahydrofuran-2-yl)-5-methylpyrimidine-2,4(1H,3H)-dione 9 (5 g, 8.082 mmol) was used following the procedure for 8 to give 10 (4 g, 80% yield).

¹H NMR (400 MHZ, CDCl₃) δ 1.39 (s, 3H), 3.41 (s, 3H), 3.44 (br d, J=1.88 Hz, 1H), 3.50-3.60 (m, 3H), 3.60-3.68 (m, 1H), 3.75-3.80 (m, 1H), 3.82 (s, 6H), 4.05-4.14 (m, 2H), 4.42-4.49 (m, 1H), 6.04 (d, J=3.75 Hz, 1H), 6.86 (br d, J=8.63 Hz, 4H), 7.24-7.36 (m, 7H), 7.43 (br d, J=7.25 Hz, 2H), 7.67 (s, 1H), 8.05 (br s, 1H); MS (ESI, m/z) calculated for [C₃₄H₃₈N₂O₉−H⁻] 617.26 found 617.31.

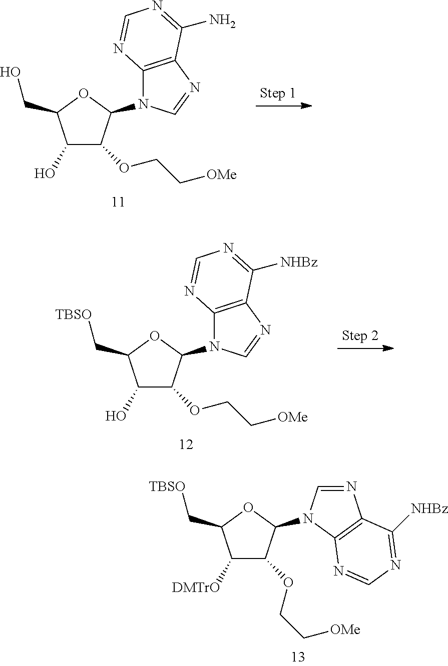

### Step 1:

### N-(9-((2R,3R,4R,5R)-5-(((tert-butyldimethylsilyl)oxy)methyl)-4-hydroxy-3-(2-methoxyethoxy)tetrahydrofuran-2-yl)-9H-purin-6-yl)benzamide 12

2′-MOE-T 11 (39 g, 119.88 mmol) and pyridine (117 mL) was concentrated in vacuo. To the above in pyridine (117 mL) was added dropwise TBS-Cl (36.1 g, 239.76 mmol) in DCM (39.0 mL) at 0° C. It was warmed up to room temperature slowly and stirred overnight. TMS-Cl (22.98 mL, 179.82 mmol) was added to the mixture at 0° C. After it was stirred at rt for 2 hours, benzoyl chloride (20.87 mL, 179.82 mmol) was added to the mixture at 0° C. and it was stirred at rt for 2 days. To the mixture was added ice at 0° C. and stirred for 1 hour. Then, aqueous ammonium hydroxide (100 mL) was added at room temperature and stirred for 1 hour. It was extracted with DCM (200 mL×2), washed with water, dried over Na₂SO₄, and concentrated. The crude was purified by a column chromatography (200 g Sfar, EtOAc/Heptane 5 to 100%) to give 12 (50 g, 77% yield).

¹H NMR (400 MHZ, CDCl₃) δ 0.00 (d, J=3.75 Hz, 6H), 0.82 (s, 9H), 3.25 (s, 3H), 3.30-3.40 (m, 1H), 3.44-3.52 (m, 1H), 3.54-3.61 (m, 1H), 3.74 (dd, J=11.57, 2.31 Hz, 1H), 3.80-3.91 (m, 2H), 4.08 (br d, J=3.13 Hz, 1H), 4.28-4.36 (m, 2H), 6.14 (d, J=4.50 Hz, 1H), 7.37-7.44 (m, 2H), 7.69 (d, J=7.25 Hz, 1H), 7.90 (br d, J=7.50 Hz, 2H), 8.29 (s, 1H), 8.69 (s, 1H), 8.85 (br s, 1H).

### Step 2:

### N-(9-((2R,3R,4R,5R)-4-(bis(4-methoxyphenyl) (phenyl) methoxy)-5-(((tert-butyldimethylsilyl)oxy)methyl)-3-(2-methoxyethoxy)tetrahydrofuran-2-yl)-9H-purin-6-yl)benzamide 13

To N-(9-((2R,3R,4R,5R)-5-(((tert-butyldimethylsilyl)oxy)methyl)-4-hydroxy-3-(2-methoxyethoxy)tetrahydrofuran-2-yl)-9H-purin-6-yl)benzamide 12 (18 g, 33.107 mmol) in pyridine (126 mL) was added 4,4′-(chloro(phenyl)methylene)bis(methoxybenzene) (16.83 g, 49.66 mmol) at room temperature and was stirred overnight at room temperature. Upon the completion, it was diluted with EtOAc (300 mL) and washed with water (150 mL). The organic layer was concentrated and purified by a column chromatography (200 g Sfar, EtOAc/Heptane 5 to 100%) to give 13 (27.6 g, 99% yield).

¹H NMR (400 MHz, CDCl₃) δ 0.00 (s, 3H), 0.03 (s, 3H), 0.85 (s, 9H), 3.25 (s, 3H), 3.31 (dd, J=11.57, 2.69 Hz, 1H), 3.45-3.52 (m, 2H), 3.52-3.59 (m, 2H), 3.67 (dd, J=11.51, 1.88 Hz, 1H), 3.80 (d, J=1.38 Hz, 6H), 3.81-3.84 (m, 1H), 3.94-3.98 (m, 1H), 4.31 (t, J=4.44 Hz, 1H), 6.44 (d, J=5.13 Hz, 1H), 6.78-6.86 (m, 4H), 7.30-7.34 (m, 1H), 7.43-7.51 (m, 5H), 7.53-7.66 (m, 5H), 7.84 (d, J=7.37 Hz, 1H), 8.05 (d, J=7.38 Hz, 2H), 8.34 (s, 1H), 8.86 (s, 1H), 9.02 (br s, 1H); MS (ESI, m/z) calculated for [C₄₇H₅₅N₅O₈Si+H⁺] 846.38 found 846.3.

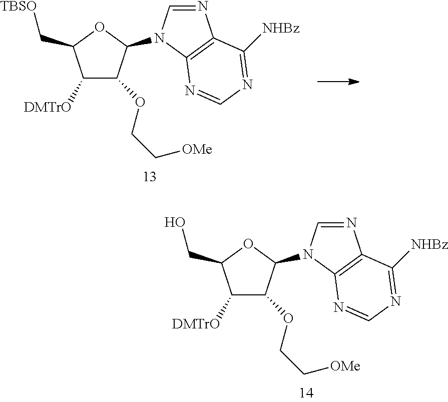

### N-(9-((2R,3R,4R,5R)-4-(bis(4-methoxyphenyl) (phenyl) methoxy)-5-(hydroxymethyl)-3-(2-methoxyethoxy)tetrahydrofuran-2-yl)-9H-purin-6-yl)benzamide 14

To N-(9-((2R,3R,4R,5R)-4-(bis(4-methoxyphenyl) (phenyl) methoxy)-5-(((tert-butyldimethylsilyl)oxy)methyl)-3-(2-methoxyethoxy)tetrahydrofuran-2-yl)-9H-purin-6-yl)benzamide 13 (15 g, 17.729 mmol) in THF (150 mL) was added pyridine (28.7 mL) and HF-Pyridine (14.29 g, 88.645 mmol) at 0° C. Then it was stirred overnight at room temperature. Upon the completion, it was quenched with saturated NaHCO₃ carefully and extracted with EtOAc. The crude was purified by a column chromatography (200 g Sfar, EtOAc/Heptane (20 to 100%) to give 14 (12.5 g, 96% yield).

¹H NMR (400 MHZ, CDCl₃) δ 2.87-2.94 (m, 1H), 2.96 (s, 3H), 3.12-3.21 (m, 1H), 3.22-3.34 (m, 3H), 3.40 (br d, J=12.88 Hz, 1H), 3.55-3.70 (m, 1H), 3.73 (s, 6H), 4.41 (d, J=4.88 Hz, 1H), 4.77 (dd, J=7.82, 5.19 Hz, 1H), 5.75 (br d, J=11.13 Hz, 1H), 6.16 (d, J=8.00 Hz, 1H), 6.78 (br d, J=8.25 Hz, 4H), 7.14-7.18 (m, 1H), 7.21-7.27 (m, 2H), 7.38-7.58 (m, 9H), 7.95 (br d, J=7.50 Hz, 2H), 8.14 (s, 1H), 8.67 (s, 1H), 8.92 (s, 1H); MS (ESI, m/z) calculated for [C₄₁H₄₁N₅O₈−H⁺] 730.30 found 730.0.

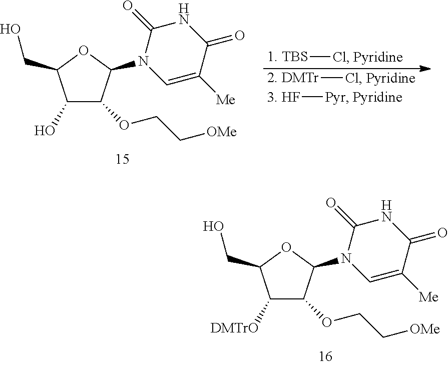

### 1-((2R,3R,4R,5R)-4-(bis(4-methoxyphenyl) (phenyl) methoxy)-5-(hydroxymethyl)-3-(2-methoxyethoxy)tetrahydrofuran-2-yl)-5-methylpyrimidine-2,4(1H,3H)-dione 16

1-((2R,3R,4R,5R)-4-hydroxy-5-(hydroxymethyl)-3-(2-methoxyethoxy)tetrahydrofuran-2-yl)-5-methylpyrimidine-2,4(1H,3H)-dione (25 g, 79.036 mmol) 15 was used following similar 3 steps used for 14 to provide 16.

¹H NMR (400 MHZ, CDCl₃) δ 1.90 (s, 3H), 3.27 (ddd, J=12.73, 7.54, 1.88 Hz, 1H), 3.40 (s, 3H), 3.56-3.71 (m, 5H), 3.77-3.80 (m, 1H), 3.82 (s, 6H), 3.82-3.87 (m, 2H), 5.76 (d, J=3.88 Hz, 1H), 6.85 (dd, J=8.76, 1.50 Hz, 4H), 7.23-7.27 (m, 1H), 7.29-7.34 (m, 2H), 7.42-7.51 (m, 5H), 7.57 (d, J=7.38 Hz, 2H), 8.06 (br s, 1H); MS (ESI, m/z) calculated for [C₃₄H₃₈N₂O₉−H⁺] 617.26 found 616.91.

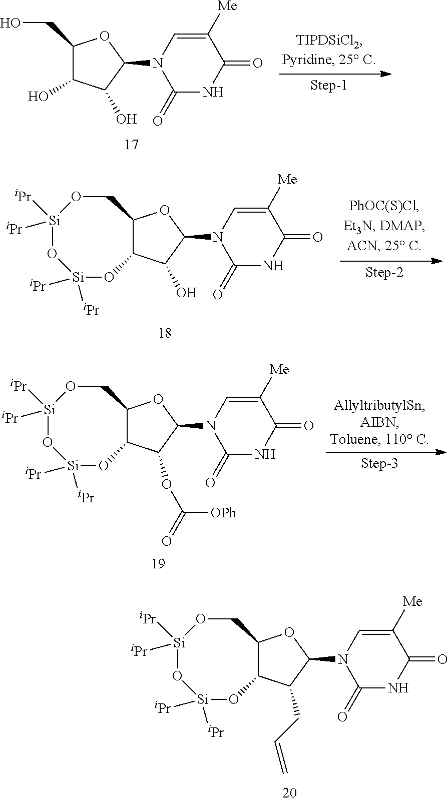 [SMILES: Cc1cn([C@@H]2O[C@H](CO)[C@@H](O)[C@H]2O)c(=O)[nH]c1=O ; Cc1cn([C@@H]2O[C@@H]3CO[Si](C(C)C)(C(C)C)O[Si](C(C)C)(C(C)C)O[C@H]3[C@H]2O)c(=O)[nH]c1=O ; Cc1cn([C@@H]2O[C@@H]3CO[Si](C(C)C)(C(C)C)O[Si](C(C)C)(C(C)C)O[C@H]3[C@H]2OC(=O)Oc2ccccc2)c(=O)[nH]c1=O ; C=CC[C@@H]1[C@@H]2O[Si](C(C)C)(C(C)C)O[Si](C(C)C)(C(C)C)OC[C@H]2O[C@H]1n1cc(C)c(=O)[nH]c1=O]

### 1-((6aR,8R,9R,9aS)-9-allyl-2,2,4,4-tetraisopropyltetrahydro-6H-furo[3,2-f][1,3,5,2,4]trioxadisilocin-8-yl)-5-methylpyrimidine-2,4(1H,3H)-dione 20

Step 1: In a round bottom flask added 5-Methyl uridine 17 (200.0 g, 0.775 mol) in pyridine (1.0 L at 25° C. under nitrogen. After 20 minutes of stirring TIPDSiCl₂ (3-Dichloro-1, 1, 3, 3-tetraisopropyl disiloxane, 1.2 eq., 0.929 mol) was added dropwise, maintaining internal temperature at 25-30° C. under nitrogen. The reaction mixture was allowed to stir at room temperature for 15-20 hours at 25-30° C. under nitrogen. The reaction mixture was quenched with water (2 L) and extracted with EtOAc (5×1 L). The combined EtOAc layers were washed with 1 N aqueous HCl solution (5×1 L) to remove pyridine completely. The combined EtOAc layers were washed with sat. sodium bicarbonate (2×1 L), water (2×1 L) and brine solution (1 L). The combined EtOAc layers were concentrated, swapped with n-heptane (5×1 L), and dried under vacuum below 45° C. to get 400.0 g of crude 18 in >100% yield.

Step 2: In a round bottom flask added 18 (400.0 g, 0.799 mole) and acetonitrile (5.2 L) at 25° C. under nitrogen. After 20 minutes of stirring, triethyl amine (155.4 mL, 1.5 eq.) was added dropwise maintaining internal temperature below 25-30° C. followed by 4-Dimethylaminopyridine (49.93 g, 0.45 eq.) portion wise at 25-30° C. under nitrogen. The reaction mixture was cooled to 0-5° C. and added O-Phenyl chlorothioformate (151.3 g, 1.1 eq.) dropwise over a period of 30 minutes at 0-5° C. under nitrogen. The reaction mixture was stirred at 0-5° C. under nitrogen for 2 hours then cooling removed, and the reaction mixture was stirred at 25-30° C. under nitrogen for 15-20 hr. After completion of the reaction, solvent was distilled under vacuum and residue was dissolved in EtOAc (4 L) and added water (3 L). The reaction mixture was stirred for 30 min. Layers were separated and aq. layer extracted with EtOAc (3×1 L). The combined EtOAc layers were washed with 1 N aq. HCl solution (5×1 L) to remove 4-Dimethylamino pyridine completely. The combined EtOAc layers was washed with sat. sodium bicarbonate (2×1 L), water (2×1 L) and brine solution (1 L). The combined EtOAc layers were concentrated, swapped with n-heptane (5×1 L), and dried under vacuum below 45° C. to get 652.0 g of crude product. The crude product (652.0 g) was purified by a column chromatography using 8% EtOAc in hexanes as a mobile phase to afford 303 g of 19.

Step 3: In a round bottom flask added 19 (300.0 g, 0.47 mol) in toluene (600 mL) at 25-30° C. under nitrogen. After 20 minutes of stirring degassed for 30 minutes with nitrogen. Allyltributyltin was added slowly at 25-30° C. under nitrogen. Then added AIBN portion wise at 25-30° C. under nitrogen. Degassed for 10 minutes with nitrogen at 25-30° C. The reaction mixture was heated to 100-110° C. and maintained at reflux for 24 hours. After completion of the reaction, the reaction mixture was cooled to 25-30° C. and solvent was distilled under vacuum to get crude product as a residue. The crude product was purified by a column chromatography using 8% EtOAc in hexanes as a mobile phase to afford 190 g of 20 (50.27% yield).

¹H NMR (400 MHZ, CDCl₃) δ ppm 9.12 (s, 1H), 7.36 (s, 1H), 5.96-5.87 (m, 1H), 5.85-5.84 (m, 1H), 5.17-5.08 (m, 2H), 4.56-4.53, (t, 1H), 4.16-4.01 (m, 3H), 3.93-3.91 (t, 1H), 2.62-2.59 (t, 1H), 2.32-2.21 (m, 1H), 2.05 (s, 1H), 1.92 (s, 3H), 1.28-1.20 (t, 2H), 1.09-1.06 (m, 25H); MS (ESI, m/z) calculated for [C₂₅H₄₄N₂O₆Si₂+H⁺] 525.27 found 524.8.

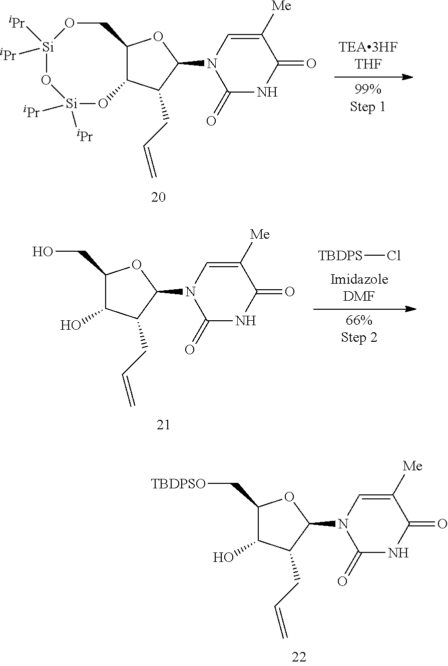

### 1-((2R,3R,4S,5R)-3-allyl-5-(((tert-butyldiphenylsilyl)oxy)methyl)-4-hydroxytetrahydrofuran-2-yl)-5-methylpyrimidine-2,4(1H,3H)-dione 21

Step 1: To a solution of 20 (202.4 g, 385.67 mmol) and TEA (117.08 g, 1.16 mol) in THF (2 L) was added triethylamine trihydrofluoride (381.75 g, 2.37 mol) and the reaction stirred for 2 h. Trimethylmethoxysilane (602.94 g, 5.79 mol) was added and the reaction stirred a further 1 h. All the solvent was removed by evaporation to afford crude 21 (159.8 g, 146% yield), which was used directly.

Step 2: To a solution of crude 21 (118.22 g, 418.78 mmol) in DMF (1.1 L) at 0° C. was added imidazole (71.28 g, 1.05 mol). A solution of TBDPSCl (120.86 g, 439.72 mmol) in DMF (100 mL) was added slowly and the reaction allowed to warm to room temperature and stirred under nitrogen overnight. Further Imidazole (28.51 g, 0.42 mol) and TBDPSCl (48.34 g, 175.89 mmol) were added and stirring continued overnight. The reaction was quenched by addition of water (8 L) and the aqueous extracted with EtOAc (1L×3), the combined organic layers were washed with brine, dried over Na₂SO₄ and concentrated. The crude product was triturated with Petroleum ether (2 L) to give 22 (166 g, 76% yield) as a white solid.

### General Procedure 1 for Phosphoramidite Activation

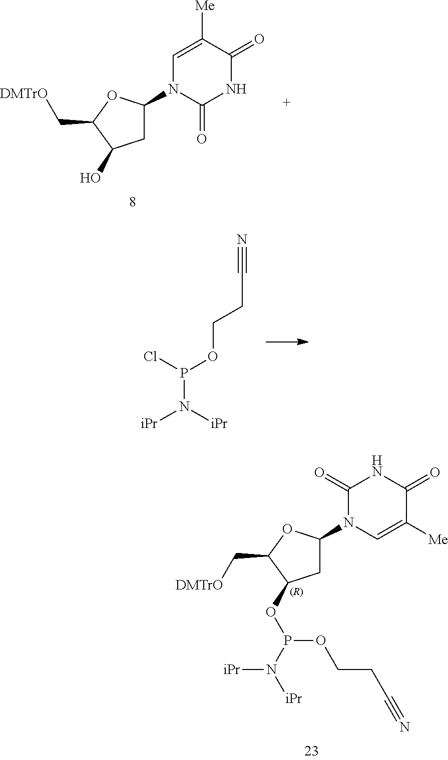

### (2R,3R,5R)-2-((bis(4-methoxyphenyl) (phenyl) methoxy)methyl)-5-(5-methyl-2,4-dioxo-3,4-dihydropyrimidin-1 (2H)-yl)tetrahydrofuran-3-yl (2-cyanoethyl)diisopropylphosphoramidite 23

1-((2R,4R,5R)-5-((bis(4-methoxyphenyl) (phenyl) methoxy)methyl)-4-hydroxytetrahydrofuran-2-yl)-5-methylpyrimidine-2,4(1H,3H)-dione 8 (1 g, 1.836 mmol) in dry DCM (6.5 mL) was added Hunig's Base (802 μl, 4.59 mmol) under nitrogen. Then, 3-((chloro(diisopropylamino)phosphaneyl)oxy) propanenitrile (614 μl, 2.754 mmol) was added dropwise at 0° C. and the mixture was stirred overnight at room temperature. MeOH (1.5 mL, 36.724 mmol) was added and after 10 minutes, the reaction mixture was concentrated in vacuo. The crude was purified by a column chromatography (50 g SiO₂, EtOAc/Heptane 5 to 80% gradient) to give 23 (1.33 g, 97% yield). The diastereomers were partially separated during purification.

Peak1: ¹H NMR (400 MHZ, CD₃CN) δ 0.97 (d, J=6.75 Hz, 6H), 1.12 (d, J=6.75 Hz, 6H), 1.72 (d, J=1.00 Hz, 3H), 2.11-2.14 (m, 1H), 2.41-2.46 (m, 2H), 2.64 (ddd, J=15.04, 7.97, 5.25 Hz, 1H), 3.31-3.45 (m, 3H), 3.48-3.65 (m, 3H), 3.79 (d, J=1.63 Hz, 6H), 4.31 (dt, J=8.13, 3.00 Hz, 1H), 4.42-4.49 (m, 1H), 6.16 (dd, J=7.88, 2.13 Hz, 1H), 6.88-6.92 (m, 4H), 7.23-7.30 (m, 1H), 7.30-7.46 (m, 7H), 7.51 (d, J=7.66 Hz, 2H), 8.89 (br s, 1H); ³¹P NMR (162 MHZ, CD₃CN) δ 147.11.

Peak 2: ¹H NMR (400 MHZ, CD₃CN) δ 0.91 (d, J=6.75 Hz, 6H), 1.09 (d, J=6.75 Hz, 6H), 1.75 (d, J=1.00 Hz, 3H), 2.30-2.37 (m, 1H), 2.54-2.58 (m, 2H), 2.58-2.63 (m, 1H), 3.23 (dd, J=10.63, 2.25 Hz, 1H), 3.38-3.49 (m, 2H), 3.49-3.60 (m, 2H), 3.67-3.76 (m, 1H), 3.78 (d, J=1.38 Hz, 6H), 4.30 (dt, J=8.35, 2.70 Hz, 1H), 4.49-4.54 (m, 1H), 6.18 (dd, J=8.07, 2.19 Hz, 1H), 6.85-6.91 (m, 4H), 7.22-7.28 (m, 1H), 7.28-7.39 (m, 6H), 7.47-7.52 (m, 3H), 8.90 (br s, 1H); ³¹P NMR (162 MHz, CD₃CN) δ 151.16.

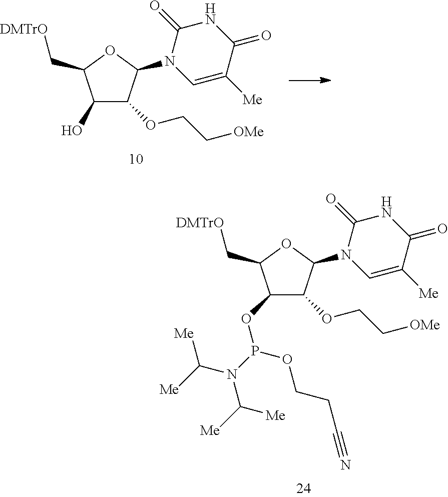

### (2R,3S,4R,5R)-2-((bis(4-methoxyphenyl) (phenyl) methoxy)methyl)-4-(2-methoxyethoxy)-5-(5-methyl-2,4-dioxo-3,4-dihydropyrimidin-1 (2H)-yl)tetrahydrofuran-3-yl (2-cyanoethyl)diisopropylphosphoramidite 24

1-((2R,3R,4S,5R)-5-((bis(4-methoxyphenyl) (phenyl) methoxy)methyl)-4-hydroxy-3-(2-methoxyethoxy)tetrahydrofuran-2-yl)-5-methylpyrimidine-2,4(1H,3H)-dione 10 (1.6 g, 2.586 mmol) was used following General Procedure 1 to give 24 (1.8 g, 85% yield) as a white form.

¹H NMR (400 MHZ, CD₃CN) δ ppm 1.06 (d, J=6.75 Hz, 3H), 1.18 (d, J=6.63 Hz, 7H), 1.38-1.44 (m, 3H), 2.06-2.13 (m, 3H), 2.52 (t, J=6.00 Hz, 1H), 2.67-2.74 (m, 1H), 3.29-3.31 (m, 3H), 3.31-3.44 (m, 2H), 3.49-3.68 (m, 5H), 3.69-3.95 (m, 9H), 4.14-4.23 (m, 1H), 4.47-4.57 (m, 1H), 5.91-5.96 (m, 1H), 6.87-6.94 (m, 4H), 7.25-7.31 (m, 1H), 7.32-7.40 (m, 6H), 7.45-7.56 (m, 3H), 8.94 (br s, 1H); ³¹P NMR (162 MHZ, CD₃CN) δ ppm 149.43, 149.60.

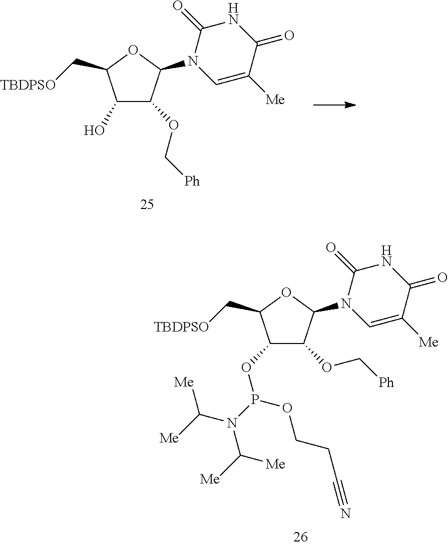

### (2R,3R,4R,5R)-4-(benzyloxy)-2-(((tert-butyldiphenylsilyl)oxy)methyl)-5-(5-methyl-2,4-dioxo-3,4-dihydropyrimidin-1 (2H)-yl)tetrahydrofuran-3-yl-(2-cyanoethyl)diisopropylphosphoramidite 26

1-((2R,3R,4R,5R)-3-(benzyloxy)-5-(((tert-butyldiphenylsilyl)oxy)methyl)-4-hydroxytetrahydrofuran-2-yl)-5-methylpyrimidine-2,4(1H,3H)-dione 25 (1.2 g, 2.05 mmol) was used following General Procedure 1 to give 26 (1.52 g, 94% yield) as a white form.

¹H NMR (400 MHZ, CD₃CN) δ ppm 1.05-1.16 (m, 12H), 1.17-1.28 (m, 9H), 1.36-1.41 (m, 3H), 2.54-2.74 (m, 2H), 3.58-3.74 (m, 2H), 3.76-4.01 (m, 4H), 4.06-4.14 (m, 1H), 4.17 (br d, J=2.75 Hz, 0.7H), 4.26 (br d, J=2.50 Hz, 0.3H), 4.53-4.62 (m, 2H), 4.73-4.84 (m, 1H), 6.05-6.10 (m, 1H), 7.08-7.20 (m, 1H), 7.26-7.35 (m, 5H), 7.40-7.53 (m, 6H), 7.65-7.74 (m, 4H), 8.87-9.02 (m, 1H);

31P NMR (162 MHZ, CD₃CN) δ ppm 149.69.

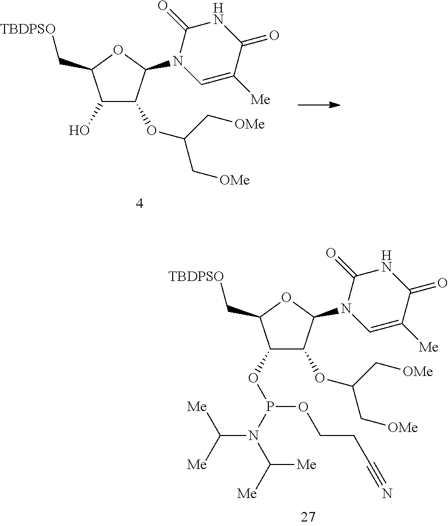

### (2R,3R,4R,5R)-2-(((tert-butyldiphenylsilyl)oxy)methyl)-4-((1,3-dimethoxypropan-2-yl)oxy)-5-(5-methyl-2,4-dioxo-3,4-dihydropyrimidin-1(2H)-yl)tetrahydrofuran-3-yl (2-cyanoethyl)diisopropylphosphoramidite 27

1-((2R,3R,4R,5R)-5-(((tert-butyldiphenylsilyl)oxy)methyl)-3-((1,3-dimethoxypropan-2-yl)oxy)-4-hydroxytetrahydrofuran-2-yl)-5-methylpyrimidine-2,4(1H,3H)-dione 4 (1.0 g, 1.67 mmol) was used following General Procedure 1 to give 27 (1.2 g, 92% yield) as a white form.

¹H NMR (400 MHZ, CD₃CN) δ ppm 1.09-1.17 (m, 12H), 1.19-1.26 (m, 9H), 1.35-1.49 (m, 3H), 2.56 (t, J=6.00 Hz, 1H), 2.66-2.76 (m, 1H), 3.15-3.27 (m, 3H), 3.30-3.34 (m, 3H), 3.34-3.55 (m, 4H), 3.62-3.76 (m, 3H), 3.77-4.04 (m, 4H), 4.12-4.27 (m, 1H), 4.38-4.44 (m, 1H), 4.48-4.56 (m, 1H), 5.99 (br d, J=6.25 Hz, 1H), 7.30-7.40 (m, 1H), 7.41-7.53 (m, 6H), 7.68-7.78 (m, 4H), 8.94 (br s, 1H); ³¹P NMR (162 MHZ, CD₃CN) δ ppm 149.49.

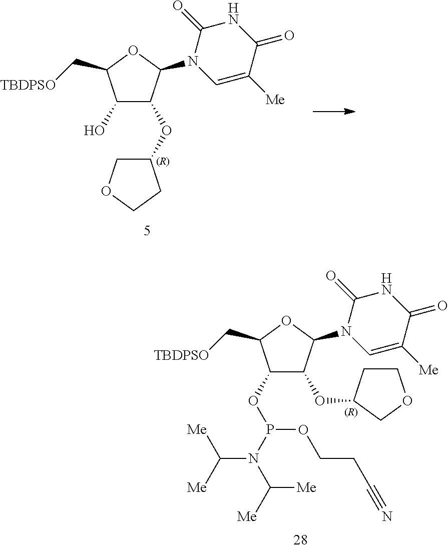

### (2R,3R,4R,5R)-2-(((tert-butyldiphenylsilyl)oxy)methyl)-5-(5-methyl-2,4-dioxo-3,4-dihydropyrimidin-1(2H)-yl)-4-(((R)-tetrahydrofuran-3-yl)oxy)tetrahydrofuran-3-yl (2-cyanoethyl)diisopropylphosphoramidite 28

1-((2R,3R,4R,5R)-5-(((tert-butyldiphenylsilyl)oxy)methyl)-4-hydroxy-3-(((R)-tetrahydrofuran-3-yl)oxy)tetrahydrofuran-2-yl)-5-methylpyrimidine-2,4(1H,3H)-dione 5 (1.1 g, 1.77 mmol) was used following General Procedure 1 to give 28 (1.1 g, 81% yield) as a white form.

¹H NMR (400 MHZ, CD₃CN) δ ppm 1.12-1.17 (m, 12H), 1.19-1.24 (m, 9H), 1.43 (s, 3H), 1.76-1.90 (m, 1H), 2.56 (t, J=5.94 Hz, 1H), 2.66-2.80 (m, 2H), 3.41-3.59 (m, 1H), 3.63-3.82 (m, 7H), 3.86-3.97 (m, 2H), 4.02-4.11 (m, 1H), 4.26 (br d, J=2.88 Hz, 0.5H), 4.37 (br dd, J=5.32, 2.69 Hz, 0.5H), 4.47-4.54 (m, 2H), 5.95-6.01 (m, 1H), 7.38 (s, 1H), 7.42-7.53 (m, 6H), 7.68-7.77 (m, 4H), 8.96 (br s, 1H); ³¹P NMR (162 MHZ, CD₃CN) δ ppm 149.30, 149.38.

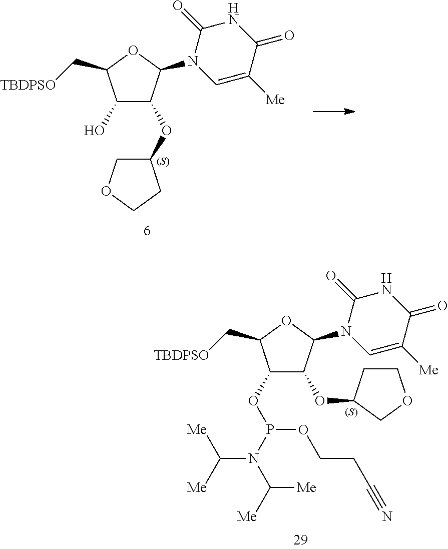

### (2R,3R,4R,5R)-2-(((tert-butyldiphenylsilyl)oxy)methyl)-5-(5-methyl-2,4-dioxo-3,4-dihydropyrimidin-1(2H)-yl)-4-(((S)-tetrahydrofuran-3-yl)oxy)tetrahydrofuran-3-yl (2-cyanoethyl)diisopropylphosphoramidite 29

1-((2R,3R,4R,5R)-5-(((tert-butyldiphenylsilyl)oxy)methyl)-4-hydroxy-3-(((S)-tetrahydrofuran-3-yl)oxy)tetrahydrofuran-2-yl)-5-methylpyrimidine-2,4(1H,3H)-dione 6 (1.2 g, 2.12 mmol) was used following General Procedure 1 to give 29 (1.4 g, 86% yield) as a white form.

¹H NMR (400 MHZ, CD₃CN) δ ppm 1.10-1.17 (m, 12H), 1.20-1.27 (m, 9H), 1.42 (s, 3H), 2.01-2.07 (m, 1H), 2.56 (t, J=6.07 Hz, 1H), 2.64-2.80 (m, 2H), 3.24 (br s, 0.5H), 3.46-3.57 (m, 0.5H), 3.61-3.69 (m, 2H), 3.71-3.75 (m, 2H), 3.78-3.87 (m, 2H), 3.87-3.97 (m, 2H), 4.02-4.09 (m, 1H), 4.13-4.18 (m, 1H), 4.24 (br d, J=3.25 Hz, 0.5H), 4.39 (br s, 0.5H), 4.46-4.53 (m, 2H), 5.93-5.99 (m, 1H), 7.36-7.40 (m, 1H), 7.40-7.53 (m, 6H), 7.68-7.77 (m, 4H), 8.99 (br s, 1H); ³¹P NMR (162 MHZ, CD₃CN) δ ppm 149.08, 149.65.

Synthesis of 2′-allyl T (allyIT) Phosphoramidite

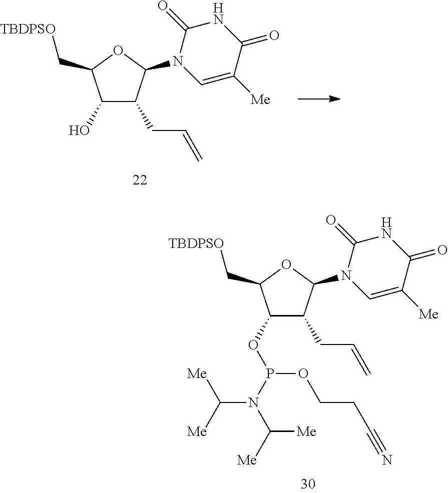

### (2R,3S,4R,5R)-4-allyl-2-(((tert-butyldiphenylsilyl)oxy)methyl)-5-(5-methyl-2,4-dioxo-3,4-dihydropyrimidin-1 (2H)-yl)tetrahydrofuran-3-yl (2-cyanoethyl)diisopropylphosphoramidite 30

1-((2R,3R,4S,5R)-3-allyl-5-(((tert-butyldiphenylsilyl)oxy)methyl)-4-hydroxytetrahydrofuran-2-yl)-5-methylpyrimidine-2,4(1H,3H)-dione 22 (3.3 g, 6.338 mmol) was used following General Procedure 1 to give 30 (3.5 g, 77% yield).

¹H NMR (400 MHZ, CD₃CN) δ ppm 1.08-1.12 (m, 9H), 1.16-1.24 (m, 12H), 1.46-1.56 (m, 3H), 2.35-2.56 (m, 3H), 2.68 (t, J=5.94 Hz, 1H), 3.60-3.75 (m, 3H), 3.73-3.97 (m, 3H), 4.20 (br s, 0.6H), 4.27 (br s, 0.4H), 4.50 (dd, J=8.76, 5.25 Hz, 0.4H), 4.62 (dd, J=11.26, 4.25 Hz, 0.6H), 5.00-5.15 (m, 2H), 5.74-5.90 (m, 1H), 6.02 (dd, J=9.01, 5.88 Hz, 1H), 7.34 (s, 1H), 7.41-7.53 (m, 6H), 7.68-7.76 (m, 4H), 8.93 (br s, 1H); ³¹P NMR (162 MHZ, CD₃CN) δ ppm 148.28, 150.69.

General Procedure 2: PSI activation (Huang, Y., et al. (2021). “AP (V) platform for oligonucleotide synthesis.” Science 373 (6560): 1265-1270)

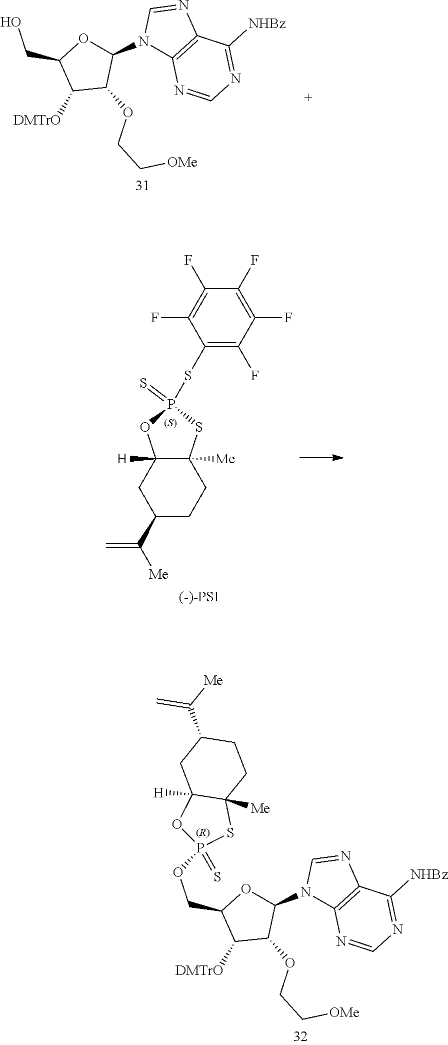

### N-(9-((2R,3R,4R,5R)-4-(bis(4-methoxyphenyl) (phenyl) methoxy)-3-(2-methoxyethoxy)-5-((((2R,3aS,6R,7aS)-3a-methyl-6-(prop-1-en-2-yl)-2-sulfidohexahydrobenzo[d][1,3,2]oxathiaphosphol-2-yl)oxy)methyl)tetrahydrofuran-2-yl)-9H-purin-6-yl)benzamide 32

N-(9-((2R,3R,4R,5R)-4-(bis(4-methoxyphenyl) (phenyl) methoxy)-5-(hydroxymethyl)-3-(2-methoxyethoxy)tetrahydrofuran-2-yl)-9H-purin-6-yl)benzamide 31 (2 g, 2.733 mmol) and (2S,3aS,6R,7aS)-3a-methyl-2-((perfluorophenyl)thio)-6-(prop-1-en-2-yl)hexahydrobenzo[d][1,3,2]oxathiaphosphole 2-sulfide (1.647 g, 3.69 mmol) ((−)-PSI reagent) were dissolved in acetonitrile (9.11 mL), and the solution was cooled in ice bath. To the mixture was added DBU (0.536 mL, 3.553 mmol) and it was stirred at 0° C. until the reaction was completed (from 0.5 to 2 hours) as monitored by UPLC-MS. The reaction mixture was diluted by EtOAc was washed with saturated NaH₂PO₄ (aq.) solution, then saturated NaHCO₃ (aq.), dried over Na₂SO₄, and purified by a silica gel chromatography (Heptane:EtOAc) to give 32 (1.82 g, 68.1% yield) a white solid.

¹H NMR (400 MHZ, CD₃CN) δ ppm 1.59-1.66 (m, 6H), 1.66-1.93 (m, 5H), 2.26 (br d, J=14.01 Hz, 1H), 2.53 (br s, 1H), 3.28 (s, 3H), 3.48-3.54 (m, 3H), 3.56-3.66 (m, 2H), 3.77 (s, 3H), 3.79 (s, 3H), 4.04-4.16 (m, 3H), 4.22 (dt, J=12.73, 3.20 Hz, 1H), 4.38 (t, J=5.13 Hz, 1H), 4.58 (s, 1H), 4.65 (s, 1H), 6.28 (d, J=3.38 Hz, 1H), 6.87 (dd, J=12.51, 9.01 Hz, 4H), 7.15-7.20 (m, 1H), 7.30-7.37 (m, 2H), 7.42 (t, J=8.50 Hz, 4H), 7.54-7.62 (m, 4H), 7.65-7.71 (m, 1H), 8.02 (br d, J=7.50 Hz, 2H), 8.23 (s, 1H), 8.67 (s, 1H), 9.27 (br s, 1H); ³¹P NMR (162 MHz, CD₃CN) δ 101.00; MS (ESI, m/z) calculated for [C₅₁H₅₆ N₅O₉PS₂+H⁺] 978.33 found 977.54.

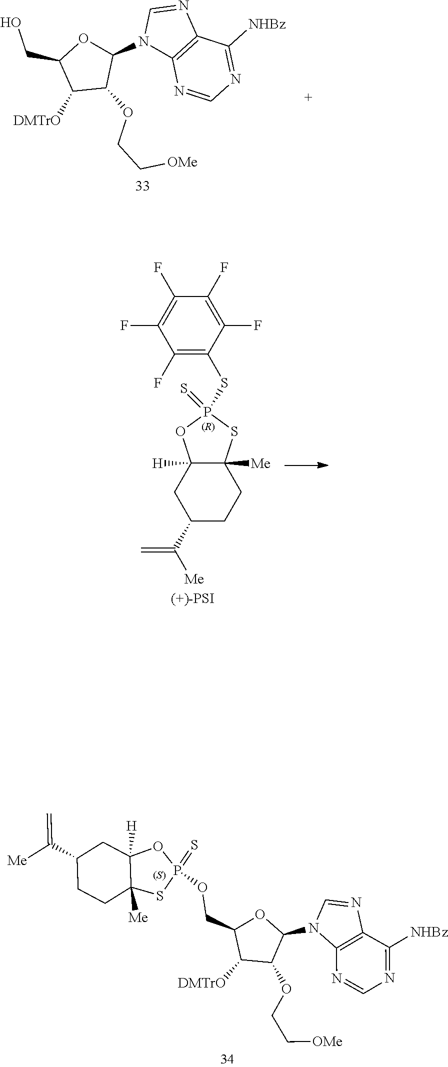

### N-(9-((2R,3R,4R,5R)-4-(bis(4-methoxyphenyl) (phenyl) methoxy)-3-(2-methoxyethoxy)-5-((((2S,3aR,6S,7aR)-3a-methyl-6-(prop-1-en-2-yl)-2-sulfidohexahydrobenzo[d][1,3,2]oxathiaphosphol-2-yl)oxy)methyl)tetrahydrofuran-2-yl)-9H-purin-6-yl)benzamide 34

N-(9-((2R,3R,4R,5R)-4-(bis(4-methoxyphenyl) (phenyl) methoxy)-5-(hydroxymethyl)-3-(2-methoxyethoxy)tetrahydrofuran-2-yl)-9H-purin-6-yl)benzamide 33 (4.2 g, 5.739 mmol) and (2R,3aR,6S,7aR)-3a-methyl-2-((perfluorophenyl)thio)-6-(prop-1-en-2-yl)hexahydrobenzo[d][1,3,2]oxathiaphosphole 2-sulfide (3.33 g, 7.461 mmol) ((+)-PSI reagent) were used following General Procedure 2 to give 34 (5.3 g, 94% yield) as a white form.

¹H NMR (400 MHZ, CD₃CN) δ ppm 1.60 (s, 3H), 1.63 (s, 3H), 1.68-1.92 (m, 4H), 1.99-2.14 (m, 2H), 2.53 (br s, 1H), 3.22 (s, 3H), 3.41-3.51 (m, 2H), 3.55-3.60 (m, 2H), 3.79 (s, 3H), 3.80 (s, 3H), 3.85 (br t, J=4.63 Hz, 1H), 3.91-4.05 (m, 3H), 4.17 (dt, J=12.57, 3.28 Hz, 1H), 4.42 (t, J=4.75 Hz, 1H), 4.71 (s, 1H), 4.77-4.80 (m, 1H), 6.27 (d, J=4.63 Hz, 1H), 6.86-6.92 (m, 4H), 7.26-7.31 (m, 1H), 7.33-7.38 (m, 2H), 7.42-7.49 (m, 4H), 7.54-7.63 (m, 4H), 7.64-7.70 (m, 1H), 8.02 (br d, J=7.50 Hz, 2H), 8.29 (s, 1H), 8.67 (s, 1H), 9.29 (br s, 1H); ³¹P NMR (162 MHZ, CD₃CN) δ 100.54; MS (ESI, m/z) calculated for [C₅₁H₅₆N₅O₉PS₂+H⁺] 978.33 found 978.1.

### Synthesis of (Rp)-RNA-A

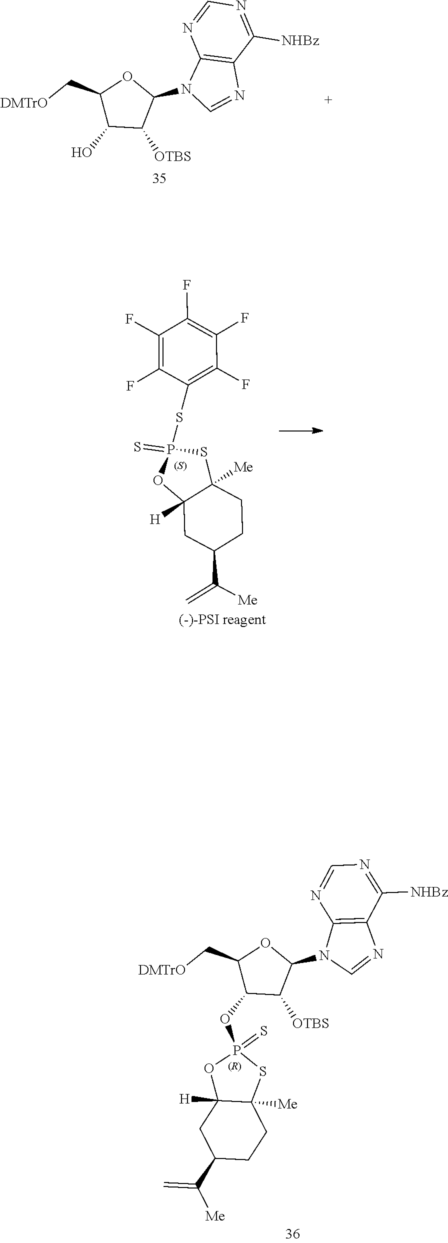

### N-(9-((2R,3R,4R,5R)-5-((bis(4-methoxyphenyl) (phenyl) methoxy)methyl)-3-((tert-butyldimethylsilyl)oxy)-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-9H-purin-6-yl)benzamide 36

N-(9-((2R,3R,4R,5R)-5-((bis(4-methoxyphenyl)(phenyl) methoxy)methyl)-3-((tert-butyldimethylsilyl)oxy)-4-hydroxytetrahydrofuran-2-yl)-9H-purin-6-yl)benzamide 35 (1 g, 1.269 mmol) and (2S,3aS,6R,7aS)-3a-methyl-2-((perfluorophenyl)thio)-6-(prop-1-en-2-yl)hexahydrobenzo[d][1,3,2]oxathiaphosphole 2-sulfide (0.765 g, 1.713 mmol) ((−)-PSI reagent) were used following General Procedure 2 to provide 36 (1.24 g, 94% yield) as a while form.

¹H NMR (400 MHZ, CD₃CN) δ ppm −0.19 (s, 3H), 0.00 (s, 3H), 0.74 (s, 9H), 1.66 (s, 3H), 1.77 (s, 3H), 1.86-1.96 (m, 4H), 2.01-2.08 (m, 1H), 2.20-2.27 (m, 1H), 2.62 (d, J=2617.88 Hz, 1H), 3.39 (dd, J=10.69, 4.44 Hz, 1H), 3.52 (dd, J=10.63, 4.13 Hz, 1H), 3.72 (s, 6H), 4.35 (q, J=4.00 Hz, 1H), 4.51 (dt, J=12.51, 3.25 Hz, 1H), 4.92 (s, 1H), 4.99-5.02 (m, 1H), 5.32 (t, J=5.44 Hz, 1H), 5.54-5.60 (m, 1H), 6.07 (d, J=6.13 Hz, 1H), 6.81 (d, J=8.63 Hz, 4H), 7.15-7.35 (m, 8H), 7.43-7.53 (m, 4H), 7.56-7.61 (m, 1H), 8.06 (br d, J=7.63 Hz, 2H), 8.40 (s, 1H), 8.51 (s, 1H), 9.89 (br s, 1H); ³¹P NMR (162 MHZ, CD₃CN) δ 100.26; MS (ESI, m/z) calculated for [C₅₄H₆₄N₅O₈PS₂Si+H⁺] 1034.37 found 1034.39.

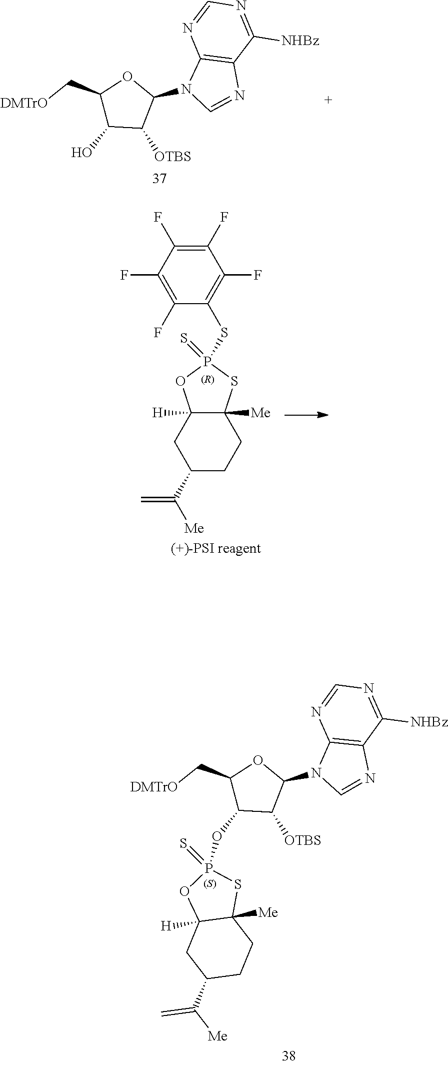

### N-(9-((2R,3R,4R,5R)-5-((bis(4-methoxyphenyl)(phenyl) methoxy)methyl)-3-((tert-butyldimethylsilyl)oxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-9H-purin-6-yl)benzamide 38

N-(9-((2R,3R,4R,5R)-5-((bis(4-methoxyphenyl)(phenyl) methoxy)methyl)-3-((tert-butyldimethylsilyl)oxy)-4-hydroxytetrahydrofuran-2-yl)-9H-purin-6-yl)benzamide 37 (3 g, 3.807 mmol) and (2R,3aR,6S,7aR)-3a-methyl-2-((perfluorophenyl)thio)-6-(prop-1-en-2-yl)hexahydrobenzo[d][1,3,2]oxathiaphosphole 2-sulfide (2.210 g, 4.949 mmol) ((+)-PSI reagent) were used following General Procedure 2 to give 38 (3.15 g, 80% yield).

¹H NMR (400 MHZ, CD₃CN) δ ppm −0.20 (s, 3H), 0.00 (s, 3H), 0.75 (s, 9H), 1.68 (s, 3H), 1.78 (s, 3H), 1.79-1.93 (m, 2H), 2.05-2.12 (m, 6H), 2.18-2.25 (m, 1H), 2.63 (br s, 1H), 3.37 (dd, J=10.76, 4.25 Hz, 1H), 3.54 (dd, J=10.69, 3.94 Hz, 1H), 3.77 (s, 6H), 4.38-4.42 (m, 1H), 4.49 (dt, J=12.73, 3.27 Hz, 1H), 4.90 (s, 1H), 5.01 (s, 1H), 5.22 (t, J=5.44 Hz, 1H), 5.52-5.58 (m, 1H), 6.03 (d, J=6.38 Hz, 1H), 6.83-6.88 (m, 4H), 7.22-7.33 (m, 3H), 7.37 (dd, J=8.76, 1.13 Hz, 4H), 7.49-7.60 (m, 4H), 7.64-7.70 (m, 1H), 8.01 (br d, J=7.50 Hz, 2H), 8.29 (s, 1H), 8.58 (s, 1H), 9.25 (s, 1H); ³¹P NMR (162 MHZ, CD₃CN) δ 102.40; MS (ESI, m/z) calculated for [C₅₄H₆₄N₅O₈PS₂Si+H⁺] 1034.37 found 1033.78.

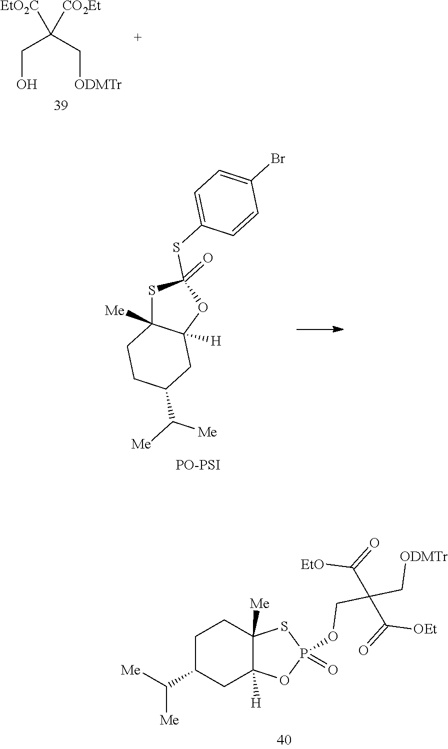

### Diethyl 2-((bis(4-methoxyphenyl)(phenyl)methoxy)methyl)-2-((((2R,3aS,6R,7aS)-6-isopropyl-3a-methyl-2-oxidohexahydrobenzo[d][1,3,2]oxathiaphosphol-2-yl)oxy)methyl)malonate 40

Diethyl 2-((bis(4-methoxyphenyl) (phenyl) methoxy)methyl)-2-(hydroxymethyl) malonate 39 (4 g, 7.654 mmol) and (2S,3aS,6R,7aS)-2-((4-bromophenyl)thio)-6-isopropyl-3a-methylhexahydrobenzo[d][1,3,2]oxathiaphosphole 2-oxide (4.19 g, 9.95 mmol) (PO-PSI reagent) were used following General Procedure 2 to give 40 (3.1 g, 54% yield) as a solid.

¹H NMR (400 MHZ, CD₃CN) δ ppm 0.91 (dd, J=13.45, 6.44 Hz, 6H), 1.19 (t, J=7.13 Hz, 6H), 1.66 (s, 4H), 1.67-1.76 (m, 3H), 2.00-2.11 (m, 3H), 3.52-3.60 (m, 2H), 3.80 (s, 6H), 4.06-4.22 (m, 6H), 4.74 (dd, J=7.00, 4.63 Hz, 2H), 6.87-6.93 (m, 4H), 7.25-7.42 (m, 9H); ³¹P NMR (162 MHZ, CD₃CN) δ 39.94.

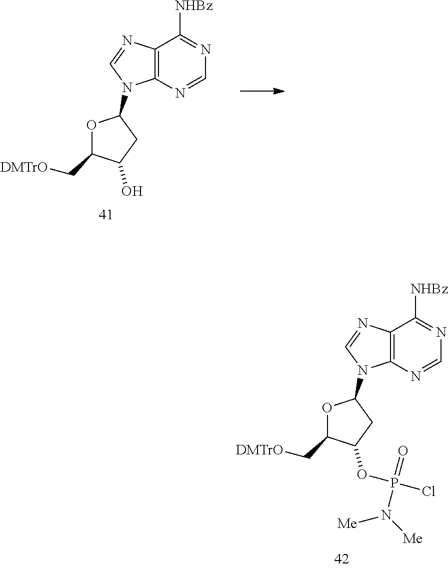

### (2R,3S,5R)-5-(6-benzamido-9H-purin-9-yl)-2-((bis(4-methoxyphenyl)(phenyl)methoxy)methyl)tetrahydrofuran-3-yl dimethylphosphoramidochloridate 42

To the suspension of N-(9-((2R,4S,5R)-5-((bis(4-methoxyphenyl) (phenyl) methoxy)methyl)-4-hydroxytetrahydrofuran-2-yl)-9H-purin-6-yl)benzamide 41 (3 g, 4.561 mmol) in MeCN (42.0 mL) and DCM (42.0 mL) was added lithium bromide (1.307 g, 15.052 mmol) at 20° C. The mixture was stirred at 20° C. for 30 min. The suspension turned to be clear solution and then gradually to flowable gel. The mixture was cooled to 10° C. before N,N-Dimethylphosphoramic dichloride (1.182 g, 7.298 mmol) was added. The reaction mixture was further cooled to 0° C., then, DBU (2.269 mL, 15.052 mmol) in MeCN (5 mL) was added slowly. After 2 hours, the reaction was worked up with saturated NaH₂PO₄ and extracted with EtOAc. The organic layer was dried over Na₂SO₄ and filtered. The solvent and volatile organics in the filtrate were removed on rotavapor. The residue was purified by a column chromatography (50 g Biotage Sfar column, EtOAc/Heptane 20 to 100%) to give 42 (2.6 g, 72.8% yield). MS (ESI, m/z) calculated for [C₄₀H₄₀ClN₆O₇P+H⁺] 783.24 found 783.3.

### <Synthesis of Oligonucleotides>

The oligonucleotides used for the synthesis of modified mRNA were synthesized using an automated nucleic acid synthesizer based on the phosphoramidite method (Table 1). Each sequence was synthesized from the 5′-end to the 3′-end by using 2′-O-TBDMS-Adenosine (N-Bz)-5′-CEP (ChemGenes #ANP3401) for the introduction of adenosine (A). The phosphorylation of the 5′-end was performed using Chemical Phosphorylation Reagent II (Glen Research #171285-25-9). Each modified structure at the 3′-end was introduced as a 3′-3′ bond by using the corresponding phosphoramidite reagent (DNA (d), 2′-Fluoro RNA (F), 2′-OMe RNA (OMe), 2′-OMe-N6-Benzyl RNA (OMe-N6-Bn), 2′-moe RNA (moe), LNA (lna), 3′-O-Butyl RNA (OBu), 2′-moeoe RNA (moeoe), 2′-C16 RNA (C16), 2′-allyl (allyl), 2′-dimethoxyisopropyl RNA (dmiPr), 2′-(R)-THF-3-yl RNA (R-THF), 2′-(S)-THF-3-yl RNA (S-THF), 2′-Benzyl RNA (Bn), 2′-deoxy Xylonucleic acid (dX), 2′-moe Xylonucleic acid (moeX)). DTTT (Chemgenes #RN-1588) was used as the sulfurizing reagent for the introduction of phosphorothioate (PS) bonds. After deprotection of each synthesized sequence according to conventional methods, they were purified using ion-pair reverse-phase HPLC. 6rA-2′-3′-idT and 6rA-2′-3′-idXT were isolated and purified by reverse-phase HPLC in the same manner as by-products generated during the synthesis of 6rA-idT and 6rA-idXT, and the structure of each sequence was confirmed by mass spectrometry. Stereoselective PS-modified nucleic acids (rp, sp) and morpholino nucleic acids (pmo) were synthesized under the following conditions.

### General Procedure 3 for Stereopure Phosphorothioate Linkage

Rp phosphorothioate linkage was obtained using Sp-PSI-monomers that were prepared from (+)-PSI reagent; Sp phosphorothioate linkage was obtained using Rp-PSI-monomers that were synthesized from (−)-PSI (Kyle W. Knouse et al., Unlocking P(V): Reagents for chiral phosphorothioate synthesis. Science 361, 1234-1238 (2018)).

Coupling: As reported in WO2022232411, PSI-monomer (0.2 mmol) in MeCN (2 mL, 0.1 M) and base solution [2,6-lutidine 1.1 mL (1 M), DBU 0.5 mL (0.3 M), MeCN 10 mL] (0.9 mL) (>10 eq. DBU and >40 eq. 2,6-utidine) was transferred to the column. It was shaken for 16 hours, it was drained and washed, and conversion was analyzed by RP HPLC-Mass after cleavage.

TentagGel-Sarcosine-imoeT-5′-resin intermediate, 41

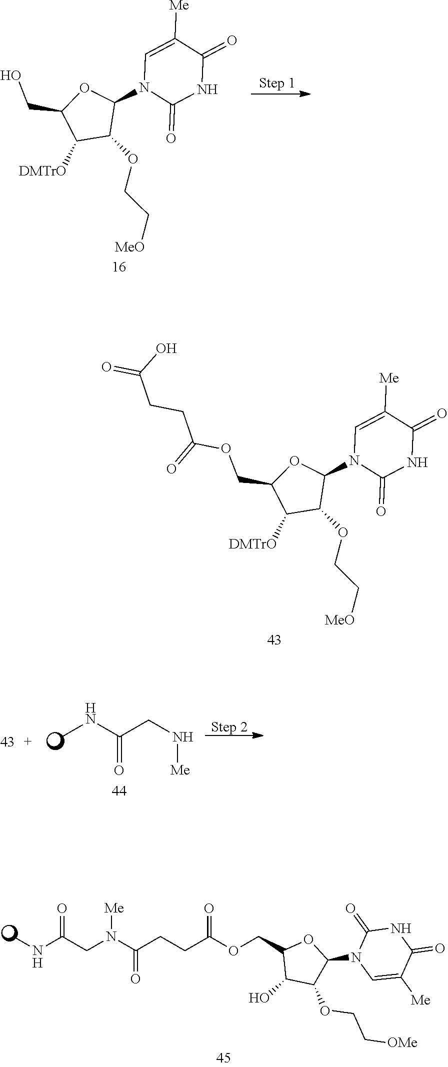

### Step 1:

### 4-(((2R,3R,4R,5R)-3-(bis(4-methoxyphenyl) (phenyl) methoxy)-4-(2-methoxyethoxy)-5-(5-methyl-2,4-dioxo-3,4-dihydropyrimidin-1 (2H)-yl)tetrahydrofuran-2-yl) methoxy)-4-oxobutanoic acid 43

To a mixture of 1-((2R,3R,4R,5R)-4-(bis(4-methoxyphenyl) (phenyl) methoxy)-5-(hydroxymethyl)-3-(2-methoxyethoxy)tetrahydrofuran-2-yl)-5-methylpyrimidine-2,4 (1H,3H)-dione 16 (4.5 g, 7.274 mmol) and succinic anhydride (0.801 g, 8.001 mmol) were added DCM (36.0 mL) and Et₃N (3.04 mL, 21.821 mmol) at room temperature.

The mixture was stirred overnight at room temperature. To the mixture was added phosphate buffer (pH 7, 6 vol) and extracted with DCM (8 vol) 3 times. Then the organic layers were dried over Na₂SO₄ and concentrated to give the crude 43.

Step 2: Solid Phase Synthesis from 3′ to 5′ direction
TentagGel-Sarcosine-imoeT-rA-OH-5′-resin intermediate 45:

To the resin 44 (up to 10 g, 2 mmol) in a 100 mL solid phase reaction flask was added 43 (4.31 g, 6 mmol) followed by (3H-[1,2,3]Triazolo[4,5-b]pyridin-3-yl)oxy)tri (pyrrolidin-1-yl)phosphonium hexafluorophosphate (V) (3.65 g, 7 mmol) in DMF (10 mL) and hunig's base (1.551 g, 12 mmol). The mixture was shaken at room temperature for 1 day and after the resin was washed with DCM (30 mL×3), it was treated for 2 minutes with 3% dichloroacetic acid (DCA) in DCM (50 mL) followed by DCM (50 mL) washing to remove the DMTr group. The process was repeated (>5 times) until no color was observed. Then the resin was washed with DCM (30 mL×3), DMF (30 mL×3) and MeCN (30 mL×3) to afford intermediate 45.

1rA-sp-imoeT:

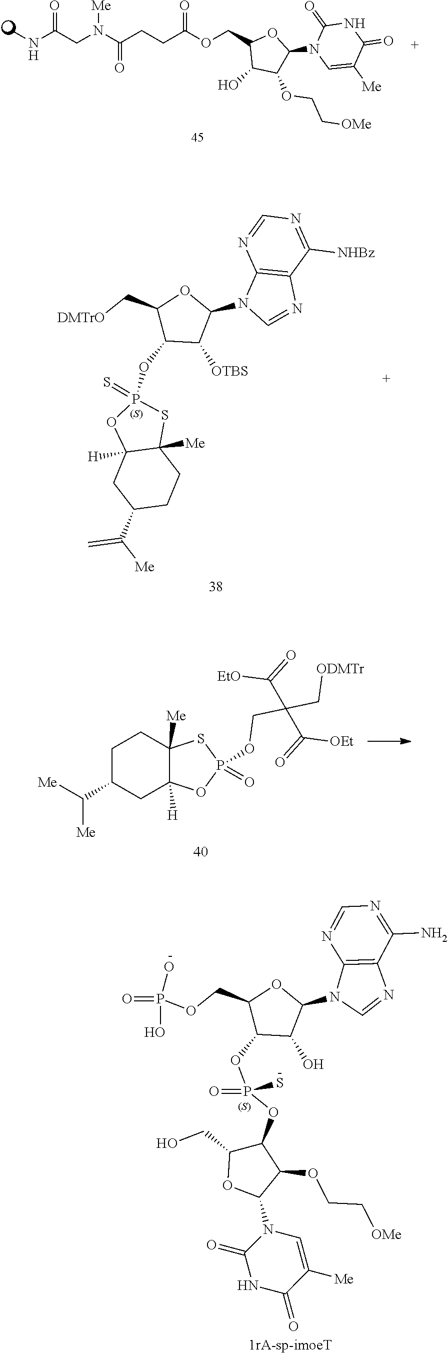

1rA-sp-imoeT was prepared from 45 with 38 (Sp-PSI-monomer) and 40 (PO-PSI-phosphorylating monomer) following General Procedure 3.

1rA-rp-imoeT:

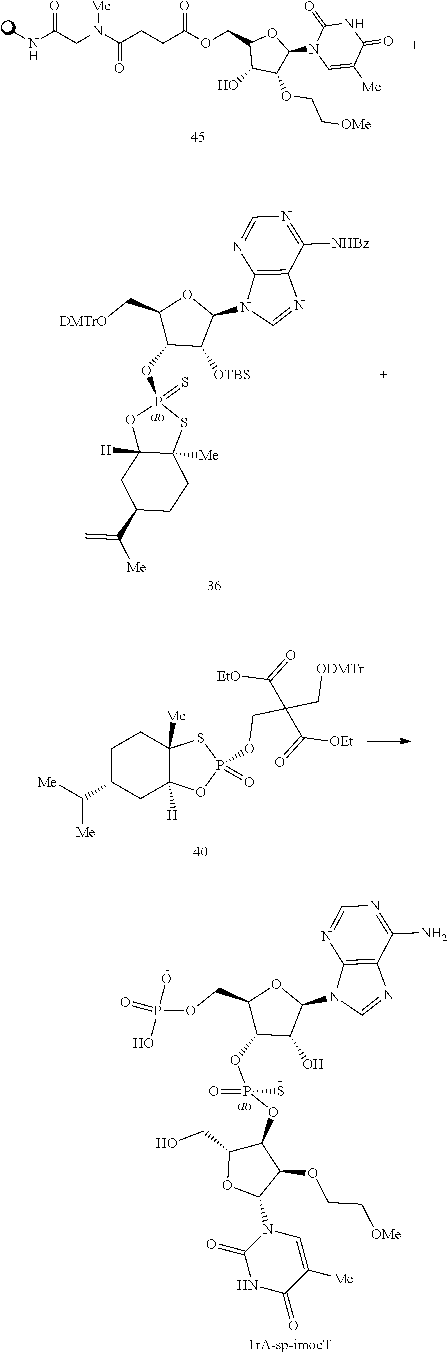

1rA-rp-imoeT was prepared from 45 with 36 (Rp-PSI-monomer) and 40 (PO-PSI-phosphorylating monomer) following General Procedure 3.

1rA-rp-rA-imoeT:

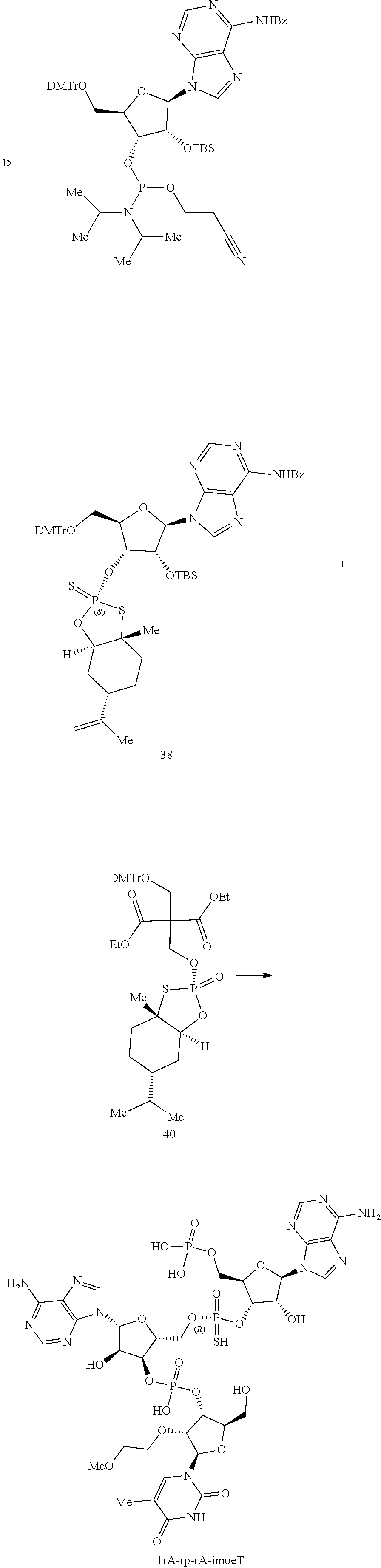

1rA-rp-rA-imoeT was prepared from 45 by coupling with 5′-DMT-2-TBS-rA-Bz-3′-amidite (Chemgene, cat #: ANP-5671) following standard amidite coupling, then, by coupling with 38 (Sp-PSI-monomer) and 40 (PO-PSI-phosphorylating monomer) following General Procedure 3.

1rA-sp-rA-imoeT:

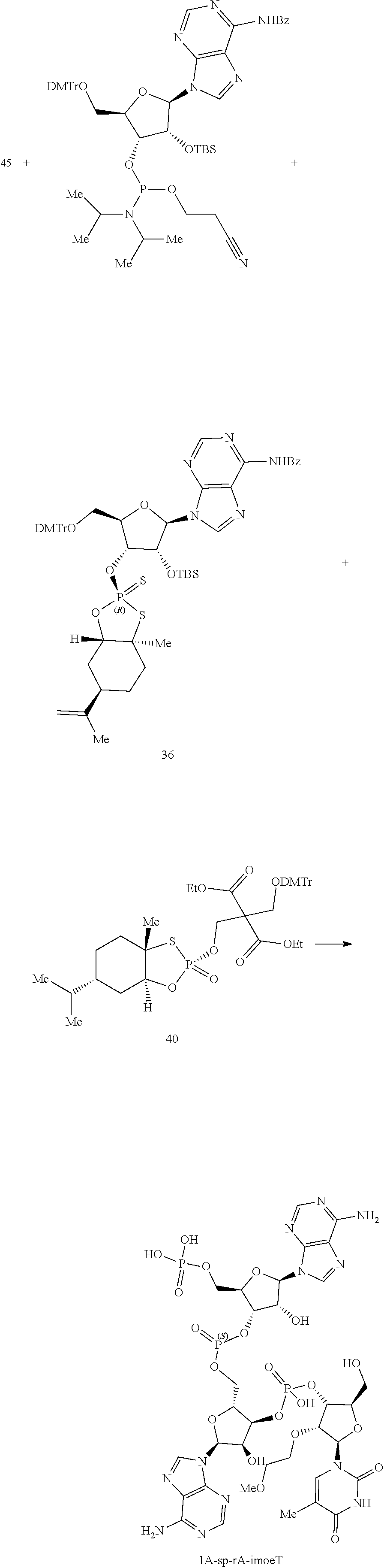

1rA-sp-rA-imoeT was prepared from 45 by coupling with 5′-DMT-2-TBS-rA-Bz-3′-amidite (Chemgene, cat #: ANP-5671) following standard amidite coupling, then, by coupling with 36 (Rp-PSI-monomer) and 40 (PO-PSI-phosphorylating monomer) following General Procedure 3.

1rA-sp-rA-sp-rA-imoeT:

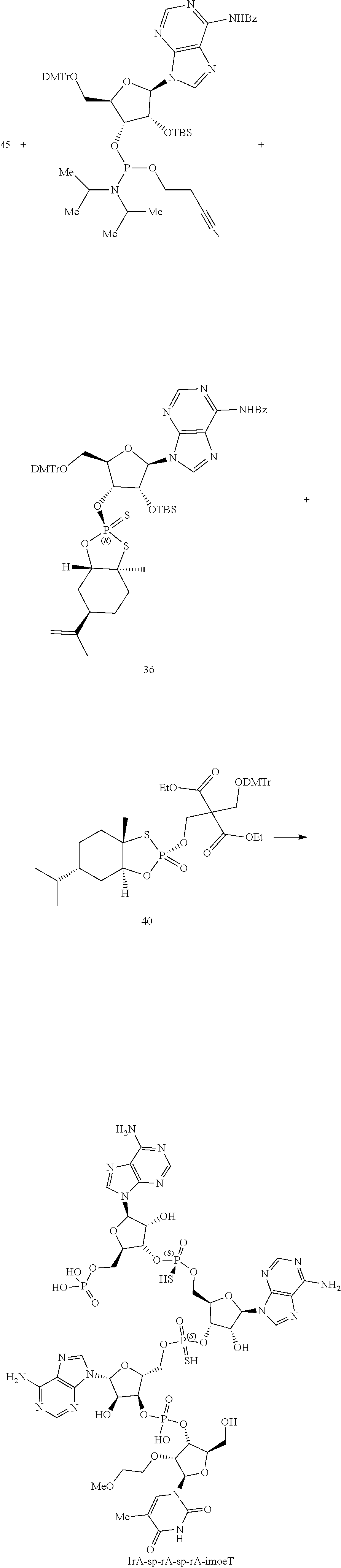

1rA-sp-rA-sp-rA-imoeT was prepared from 45 with 5′-DMT-2-TBS-rA-Bz-3′-amidite (Chemgene, cat #: ANP-5671), 36 (Rp-PSI-monomer), and 40 (PO-PSI-phosphorylating monomer) following standard amidite coupling and General Procedure 3 according to the sequence.

1rA-sp-rA-sp-rA-sp-rA-imoeT:

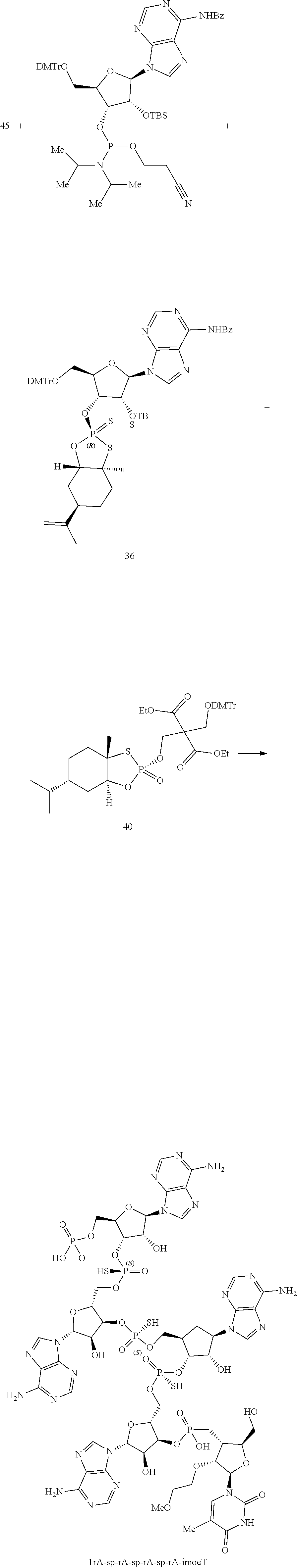

1rA-sp-rA-sp-rA-sp-rA-imoeT was prepared from 45 with 5′-DMT-2-TBS-rA-Bz-3′-amidite (Chemgene, cat #: ANP-5671), 36 (Rp-PSI-monomer), and 40 (PO-PSI-phosphorylating monomer) following standard amidite coupling and General Procedure 3 according to the sequence.

### General Procedure 4: Solid Phase Synthesis for PMO

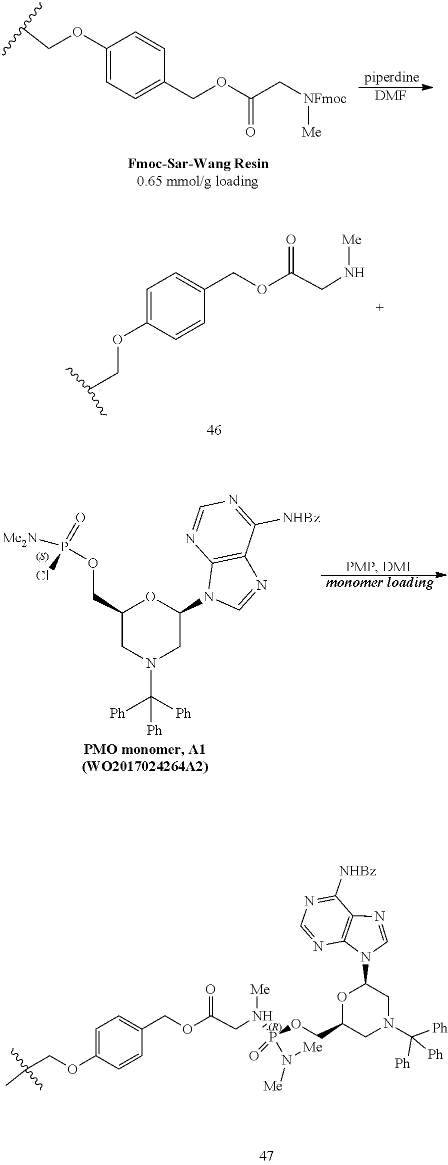

### Deprotection of Fmoc on Sar-Wang Resin:

Fmoc-SAR-Wang resin (purchased from Aapptec, RWG103, Lot #9953380, 0.65 mmol/g, 110-200 mesh) (0.25 g, 0.16 mmol) in an empty 12 mL syringe column was treated with DMF (8 mL), allowed resin to swell for 2 hours and drained DMF. The resin was treated with 20% piperidine in DMF (6 mL), shaken for 3 minutes, removed solvent, and dried for 1 minute under N₂ gas (repeated the same sequence for 4 times). Finally, the resin was washed with DMF (5 mL×5 times), washed with CH₂Cl₂ (5 mL×5 times).

### General Procedure for Solid-Phase Synthesis of PMOs:

The Fmoc deprotected resin (250 mg, 162 μmol, 0.650 mmol/g) was washed with CH₂Cl₂ (20 mL×5 times), washed with acetonitrile (20 mL×5 times), and dried. PMO monomer (1.3 eq.) was loaded to the column as a solid. Then, 20% 1,2,2,6,6-pentamethylpiperidine (PMP, >10.0 eq.) in anhydrous 1,3-dimethyl-2-imidazolidinone (DMI, up to 3 mL) were added to vessel and shaken at room temperature for 20 hours. Then, steps 5-9 in Table 1 were performed. The synthesis had a series of iterative steps including deprotection/neutralization/coupling/capping.

TABLE 1
Steps in solid-phase PMO synthesis

| Step | Reaction | Reagent | |
|---|---|---|---|
| 1 | Detritylation | 2% 3-cyanopyridinc-TFA, | 7 cycles |
| | | 0.9% EtOH, 20% | |
| | | TFE/DCM (80 mL) then | |
| | | CH₂Cl₂ (15 mL × 2) | |
| 2 | Neutralization | 10% Hunig′s base in NMP | 30 mL × |
| | | | 4 times |
| 3 | Wash | 1,3-dimethyl-2-imidazolidinone | |
| | | (30 mL × 2 times) | |
| | | MeCN (25 mL × 10 times), | |
| | | CH₂Cl₂ (25 mL × 15 times) | |
| 4 | Coupling | Monomer (1.3 eq), DMI (0.05M), | 20 h |
| | | PMP (10 eq.) | |
| 5 | Wash | MeCN (25 mL × 10 times), | |
| | | CH₂Cl₂ (25mL × 10 times) | |
| 6 | Capping | 2,6-Lutidine (30% in MeCN, 6 mL) | 3 times |
| | | Ac₂O (20% in MeCN, 6 mL) | |
| 7 | Wash | CH₂Cl₂ (25 mL × 10 times) | |
| 8 | Ac₂O | 10% Morpholine in NMP | 40 mL × |
| | removal | | 4 times |
| 9 | Wash | CH₂Cl₂ (30 mL × 10 times) | |

5rA-dA-rp-ipmoA and 5rA-dA-sp-ipmoA:

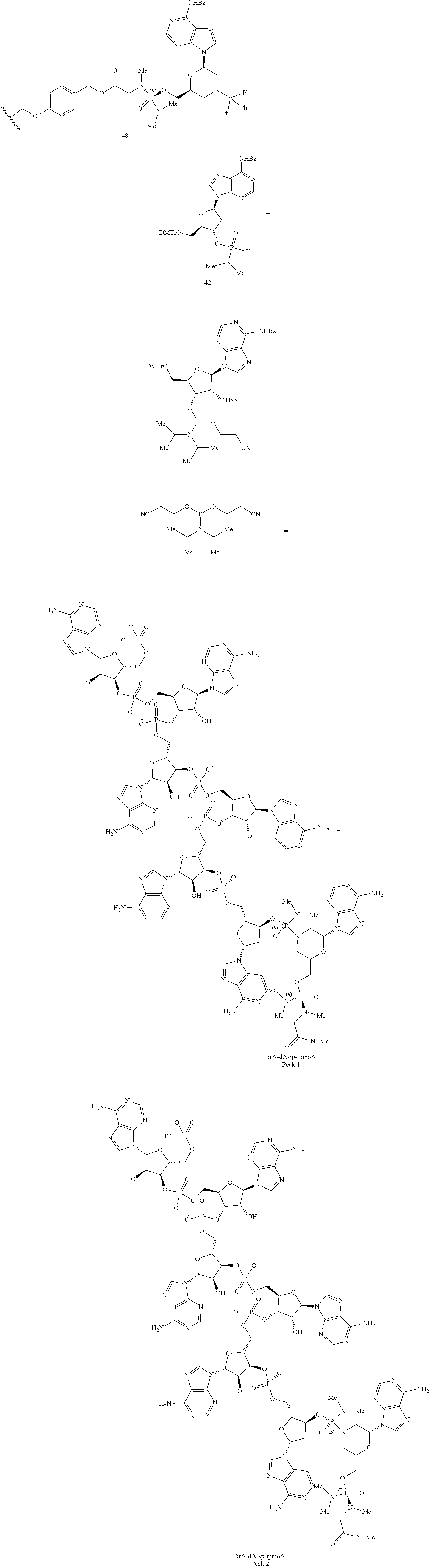

5rA-dA-rp-ipmoA and 5rA-dA-sp-ipmoA were prepared from 48 with 42, 5′-DMT-2-TBS-rA-Bz-3′-amidite (Chemgene, cat #: ANP-5671), and Bis(2-cyanoethyl)-N,N-diisopropylphosphoramidite (Cas #102690-88-0) following General Procedure 4 and standard amidite coupling according to the sequence. The crude was purified by Ion-pairing RP HPLC (from 2.5 to 22% Buffer B gradient), stereoisomers were assigned randomly (5rA-dA-rp-ipmoA for Peak 1 and 5rA-dA-sp-ipmoA for Peak 2).

The structure of each purified oligonucleotide was confirmed by an ESI-MS system (Waters Xevo G2-XS QTof). The results are shown in Table 2-1, Table 2-2, and Table 2-3.

TABLE 2-1
p indicates 5′-phosphorylation. i indicates a 3′-3′ bond.

| Compound name | sequence | Calcd. MS | Observed MS |
|---|---|---|---|
| 6rA-idA | 5′-pAAAAAA-idA-3′ | 2305.4 | 1153.8 |
| | | | [M + 2H]²⁺ |
| 6rA-idG | 5′-pAAAAAA-idG-3′ | 2321.4 | 1161.8 |
| | | | [M + 2H]²⁺ |
| 6rA-idC | 5′-pAAAAAA-idC-3′ | 2281.4 | 1142.1 |
| | | | [M + 2H]²⁺ |
| 6rA-idT | 5′-pAAAAAA-idT-3′ | 2296.4 | 2295.4 |
| | | | [M − H]⁻ |
| 6rA-iFA | 5′-pAAAAAA-iFA-3′ | 2323.4 | 2322.2 |
| | | | [M − H]⁻ |
| 6rA-iFU | 5′-pAAAAAA-iFU-3′ | 2300.3 | 2299.8 |
| | | | [M − H]⁻ |
| 6rA-iOMeA | 5′-pAAAAAA-iOMeA-3′ | 2335.4 | 2334.2 |
| | | | [M − H]⁻ |
| 6rA-iOMeU | S′-pAAAAAA-iOMeU-3′ | 2312.4 | 2311.2 |
| | | | [M − H]⁻ |
| 6rA-imoeA | 5′-pAAAAAA-imocA-3′ | 2379.4 | 1190.8 |
| | | | [M + 2H]²⁺ |
| 6rA-imoeG | 5′-pAAAAAA-imoeG-3′ | 2395.4 | 1198.7 |
| | | | [M + 2H]²⁺ |
| 6rA-imoemC | 5′-pAAAAAA-imoemC-3′ | 2369.4 | 1186.0 |
| | | | [M + 2H]²⁺ |
| 6rA-imoeT | 5′-pAAAAAA-imoeT-3′ | 2370.4 | 1186.3 |
| | | | [M + 2H]²⁺ |
| 6rA-ps-imoeA | 5′-pAAAAAA-ps-imoeA- | 2395.4 | 2394.6 |
| | 3′ | | [M − H]⁻ |
| 6rA-ilnaA | 5′-pAAAAAA-ilnaA-3′ | 2333.4 | 1167.8 |
| | | | [M + 2H]²⁺ |
| 6rA-ilnaT | 5′-pAAAAAA-ilnaT-3′ | 2324.4 | 1163.2 |
| | | | [M + 2H]²⁺ |
| 1rA-idT | 5′-pA-idT-3′ | 651.1 | 650.1 |
| | | | [M − H]⁻ |
| 1rA-imoeT | 5′-pA-imoeT-3′ | 725.1 | 724.1 |
| | | | [M − H]⁻ |
| 1rA-ps-imoeT | 5′-pA-ps-imoeT-3′ | 741.1 | 740.1 |
| | | | [M − H]⁻ |
| 6rA-2′-3′-dT | 5′-pAAAAAA-2′-3′-dT-3′ | 2296.4 | 1149.2 |
| | | | [M + 2H]²⁺ |

TABLE 2-2

| | | | |
|---|---|---|---|
| 6rA-ps2-idT | 5′-pAAAAAA-ps2-id′T-3′ | 2328.3 | 1164.9 |
| | | | [M + 2H]²⁺ |
| 6rA-idXT | 5′-pAAAAAA-idXT-3′ | 2296.4 | 1149.2 |
| | | | [M + 2H]²⁺ |
| 6rA-2′-3′-dXT | 5′-pAAAAAA-2′-3′-dXT- | 2296.4 | 1149.2 |
| | 3′ | | [M + 2H]²⁺ |
| 6rA-ps-idXT | 5′-pAAAAAA-ps-idXT-3′ | 2312.4 | 1157.2 |
| | | | [M + 2H]²⁺ |
| 5rA-dA-rp- | 5′-pAAAAA-dA-rp- | 2506.5 | 1254.4 |
| ipmoA | ipmoA-3′ | | [M + 2H]²⁺ |
| 5rA-dA-sp- | 5′-pAAAAA-dA-sp- | 2296.4 | 1254.4 |
| ipmoA | ipmoA-3′ | | [M + 2H]²⁺ |
| 1rG-imoeT | 5′-pG-imoeT-3′ | 741.1 | 740.2 |
| | | | [M − H]⁻ |
| 1rC-imoeT | 5′-pC-imoeT-3′ | 701.1 | 700.2 |
| | | | [M − H]⁻ |
| 1rU-imoeT | 5′-pU-imoeT-3′ | 702.1 | 701.2 |
| | | | [M − H]⁻ |
| 1dT-imoeT | 5′-p-dT-imoeT-3′ | 700.1 | 699.1 |
| | | | [M − H]⁻ |
| 6rA-iC16A | 5′-pAAAAAA-iC16A-3′ | 2545.6 | 1271.8 |
| | | | [M − 2H]²⁻ |
| 6rA-iallylT | S′-pAAAAAA-iallylT-3′ | 2336.4 | 1168.9 |
| | | | [M + 2H]²⁺ |
| 6rA-imoeoeA | 5′-pAAAAAA-imoeoeA- | 2423.5 | 1211.3 |
| | 3′ | | [M − 2H]²⁻ |
| 2(1rA-imoeT) | 5′-pA-imoeT-A-imoeT-3′ | 1432.3 | 1431.2 |
| | | | [M − H]⁻ |
| 1rA-2-imoeT | 5′-pA-imoeT-imoeT-3′ | 1103.2 | 1102.2 |
| | | | [M − H]⁻ |
| AGC-imoeT | 5′-pAGC-imoeT-3′ | 1375.2 | 1374.1 |
| | | | [M − H]⁻ |
| 1rA-idmiPrT | 5′-pA-idmiPrT-3′ | 769.2 | 770.4 |
| | | | [M + H]⁺ |
| 1rA-i2′-R-THFT | 5′-pA-i2′-R-THFT-3′ | 737.2 | 738.4 |
| | | | [M + H]⁺ |
| 1rA-i2′-S-THFT | 5′-pA-i2′-S-THFT-3′ | 737.2 | 738.3 |
| | | | [M + H]⁺ |
| 6rA-3′-2′-OBuA | 5′-pAAAAAA-3′-2′- | 2377.4 | 1189.7 |
| | OBuA-3′ | | [M + 2H]²⁺ |

TABLE 2-3

| | | | |
|---|---|---|---|
| IrA-iOMeA-N6- | 5′-pA-iOMeA-N6-Bn-3′ | 780.2 | 781.2 |
| Bn | | | [M + H]⁺ |
| 1rA-iBnT | 5′-pA-iBnT-3′ | 757.1 | 758.3 |
| | | | [M + H]⁺ |
| 1rA-rp-imoeT | 5′-pA-ps(Rp)-imoe′T-3′ | 741.1 | 740.2 |
| | | | [M − H]⁻ |
| 1rA-sp-imoeT | 5′-pA-ps(Sp)-imoeT-3′ | 741.1 | 740.2 |
| | | | [M − H]⁻ |
| 1rA-rp-rA-imoeT | 5′-pA-ps(Rp)-rA-imoeT-3′ | 1070.2 | 1069.3 |
| | | | [M − H]⁻ |
| 1rA-sp-rA-imoeT | 5′-pA-ps(Sp)-rA-imoeT-3′ | 1070.2 | 1069.3 |
| | | | [M − H]⁻ |
| 1rA-sp-rA-sp-rA- | 5′-pA-ps(Sp)-rA-ps(Sp)- | 1415.2 | 1414.4 |
| imoeT | rA-imoeT-3′ | | [M − H]⁻ |
| 1rA-sp-rA-sp-rA- | 5′-pA-ps(Sp)-pA-ps(Sp)- | 1760.2 | 1759.8 |
| sp-rA-imoeT | rA-ps(Sp)-rA-imoeT-3′ | | [M − H]⁻ |
| 6rA-imoeXT | 5′-pAAAAAA-imoeXT-3′ | 2370.4 | 1184.5 |
| | | | [M − 2H]²⁻ |

### <Preparation of Template DNA>

To prepare the template plasmid DNA used for in vitro transcription (IVT), a plasmid having a DNA fragment in which the T7 promoter sequence, 5′-UTR sequence, KOZAK sequence, each ORF sequence, and 3′-UTR sequence are sequentially linked was synthesized. To 1 μg of the plasmid dissolved in Nuclease-free water (210 μL), Q5 Hot Start High-Fidelity 2× Master Mix (250 μL, NEB #M0494L), 10 μM sense primer (20 μL), and 10 μM antisense primer containing poly-T (20 μL) were added. After incubation at 95° C. for 1 minute, 35 cycles of 95° C. for 30 seconds, 60° C. for 30 seconds, and 72° C. for 3 minutes were performed, followed by incubation at 72° C. for 5 minutes to amplify the template DNA by PCR. After the reaction, isopropanol (500 μL, FujiFilm catalog #166-04836) was mixed, and the mixture was left standing at −20° C. for more than 1 hour. After centrifugation (4° C., 15000 rpm, 30 minutes), the supernatant was discarded. Then, 75% ethanol was added, and after centrifugation (4° C., 15000 rpm, 5 minutes), the supernatant was discarded. This operation was repeated three times. The obtained precipitate was dissolved in Nuclease-free water to obtain the template DNA having the desired ORF sequence.

### <Preparation of Firefly Luciferase (Fluc)-mRNA>

Using each obtained template DNA, mRNA was prepared by in vitro transcription (IVT). The following components were mixed: 500 μg/mL template DNA (48 μL), 100 mM CleanCap AG (32 μL, TriLink catalog #N-7113), 100 mM ATP (40 μL, TriLink catalog #N-1510), 100 mM CTP (40 μL, TriLink catalog #N-1511), 100 mM GTP (40 μL, TriLink catalog #N-1512), 100 mM N1-methyl-y-Uridine-5′-Triphosphate (40 μL), UltraPure DNase/RNase-Free Distilled Water (437.6 μL, Thermo Fisher catalog #10977015), T7 Transcription 10× buffer (80 μL), RNase inhibitor (20 μL, NEB, catalog #M0314L), Yeast Inorganic pyrophosphatase (16 μL, NEB, catalog #M2403L), and T7 RNA Polymerase (6.4 μL, Roche catalog #08140669103). The mixture was incubated at 37° C. for 3 hours. RNase-Free DNase I (24 μL, TaKaRa catalog #2270A) was added and incubated at 37° C. for 30 minutes. Then, 10× phosphatase buffer (96 μL, NEB, catalog #B0289S) and Antarctic phosphatase (48 μL, NEB, catalog #M0289L) were added and incubated at 37° C. for 30 minutes. 8M LiCl solution (484 μL, Sigma-Aldrich catalog #L7026) was added, and the mixture was left standing at −20° C. for more than 1 hour. After centrifugation (4° C., 15000 rpm, 30 minutes), the supernatant was discarded. Then, 75% ethanol was added, and after centrifugation (4° C., 15000 rpm, 5 minutes), the supernatant was discarded. This operation was repeated three times. The obtained precipitate was dissolved in Nuclease-free water. Thus, mRNA expressing the Fluc protein (Sequence ID No. 1, Table 3) was obtained.

### <Preparation of Human Erythropoietin (EPO)-mRNA>

Using each obtained template DNA, mRNA was prepared by in vitro transcription (IVT). The following components were mixed: 300 μg/mL template DNA (160 μL), 100 mM CleanCap AG (64 μL, TriLink catalog #N-7113), 100 mM ATP (80 μL, TriLink catalog #N-1510), 100 mM CTP (80 μL, TriLink catalog #N-1511), 100 mM GTP (80 μL, TriLink catalog #N-1512), 100 mM N1-methyl-ψ-Uridine-5′-Triphosphate (80 μL), UltraPure DNase/RNase-Free Distilled Water (811.2 μL, Thermo Fisher catalog #10977015), T7 Transcription 10× buffer (160 μL), RNase inhibitor (40 μL, NEB, catalog #M0314L), Yeast Inorganic pyrophosphatase (32 μL, NEB, catalog #M2403L), and T7 RNA Polymerase (12.8 μL, Roche catalog #08140669103). The mixture was incubated at 37° C. for 3 hours. RNase-Free DNase I (48 μL, TaKaRa catalog #2270A) was added and incubated at 37° C. for 30 minutes. Then, 10× phosphatase buffer (96 μL, NEB, catalog #B0289S) and Antarctic phosphatase (48 μL, NEB, catalog #M0289L) were added and incubated at 37° C. for 30 minutes. The reaction mixture was divided into two tubes, and 8M LiCl solution (484 μL, Sigma-Aldrich catalog #L7026) was added to each. The mixtures were left standing at −20° C. for more than 1 hour. After centrifugation (4° C., 15000 rpm, 30 minutes), the supernatant was discarded. Then, 75% ethanol was added, and after centrifugation (4° C., 15000 rpm, 5 minutes), the supernatant was discarded. This operation was repeated three times. The obtained precipitate was dissolved in Nuclease-free water. Thus, mRNA expressing the EPO protein (Sequence ID No. 2, Table 4) was obtained.

TABLE 3
<Seq ID 1> Fluc-mRNA(1921 bp: 5′-UTR +
ORF + 3′-UTR + PolyA(120 nt))

| | |
|---|---|
| 5′ | AGGGAAAUAAGAGAGAAAAGAAGAGUAAGAAGAAAUAUAA |
| -UTR | GAGCCACC |
| ORF | AUGGAGGACGCCAAGAACAUCAAGAAGGGCCCCGCCCCCU |
| | UCUACCCCCUGGAGGACGGCACCGCCGGCGAGCAGCUGCA |
| | CAAGGCCAUGAAGCGGUACGCCCUGGUGCCCGGCACCAUC |
| | GCCUUCACCGACGCCCACAUCGAGGUGGACAUCACCUACG |
| | CCGAGUACUUCGAGAUGAGCGUGCGGCUGGCCGAGGCCAU |
| | GAAGCGGUACGGCCUGAACACCAACCACCGGAUCGUCGUG |
| | UGCAGCGAGAACAGCCUGCAGUUCUUCAUGCCCGUGCUGG |
| | GCGCCCUGUUCAUCGGCGUGGCCGUGGCCCCCGCCAACGA |
| | CAUCUACAACGAGCGGGAGCUGCUGAACAGCAUGGGCAUC |
| | AGCCAGCCCACCGUGGUGUUCGUGAGCAAGAAGGGCCUGC |
| | AGAAGAUCCUGAACGUGCAGAAGAAGCUGCCCAUCAUCCA |
| | GAAGAUCAUCAUCAUGGACAGCAAGACCGACUACCAGGGC |
| | UUCCAGAGCAUGUACACCUUCGUGACCAGCCACCUGCCCC |
| | CCGGCUUCAACGAGUACGACUUCGUGCCCGAGAGCUUCGA |
| | CCGGGACAAGACCAUCGCCCUGAUCAUGAACAGCAGCGGC |
| | AGCACCGGCCUGCCCAAGGGCGUGGCCCUGCCCCACCGGA |
| | CCGCCUGCGUGCGGUUCAGCCACGCCCGGGACCCCAUCUU |
| | CGGCAACCAGAUCAUCCCCGACACCGCCAUCCUGAGCGUG |
| | GUGCCCUUCCACCACGGCUUCGGCAUGUUCACCACCCUGG |
| | GCUACCUGAUCUGCGGCUUCCGGGUGGUGCUGAUGUACCG |
| | GUUCGAGGAGGAGCUGUgGGAGCCUGCAGGACUACAAGAU |
| | CCAGAGCGCCCUGCUGGUGCCCACCCUGUUCAGCUUCUUC |
| | GCCAAGAGCACCCUGAUCGACAAGUACGACCUGAGCAACC |
| | UGCACGAGAUCGCCAGCGGCGGCGCCCCCCUGAGCAAGGA |
| | GGUGGGCGAGGCCGUGGCCAAGCGGUUCCACCUGCCCGGC |
| | AUCCGGCAGGGCUACGGCCUGACCGAGACCACCAGCGCCA |
| | UCCUGAUCACCCCCGAGGGCGACGACAAGCGGGGCGCCGU |
| | GGGCAAGGUGGUGCCCUUCUUCGAGGCCAAGGUGGUGGAC |
| | CUGGACACCGGCAAGACCCUGGGCGUGAACCAGCGGGGCG |
| | AGCUGUGCGUGCGGGGCCCCAUGAUCAUCAGCGGCUACGU |
| | GAACAACCCCGAGGCCACCAACGCCCUGAUCGACAAGGAC |
| | GGCUGGCUGCACAGCGGCGACAUCGCCUACUGGGACGAGG |
| | ACGAGCACUUCUUCAUCGUGGACCGGCUGAAGAGCCUGAU |
| | CAAGUACAAGGGCUACCAGGUGGCCCCCGCCGAGCUGGAG |
| | AGCAUCCUGCUGCAGCACCCCAACAUCUUCGACGCCGGCG |
| | UGGCCGGCCUGCCCGACGACGACGCCGGCGAGCUGCCCGC |
| | CCCCGUGGUGGUGCUGGAGCACGGCAAGACCAUGACCGAG |
| | AAGGAGAUCGUGGACUACGUGGCCAGCCAGGUGACCACCG |
| | CCAAGAAGCUGGGGGGCGGCGUGGUGUUCGUGGACGAGGU |
| | GCCCAAGGGCCUGACCGGCAAGCUGGACGCCCGGAAGAUC |
| | CGGGAGAUCCUGAUCAAGGCCAAGAAGGGCGGCAAGAUCG |
| | CCGUGUGA |
| 3′- | UAAGCUGCCUUCUGCGGGGCUUGCCUUCUGGCCAUGCCCU |
| UTR | UCUUCUCUCCCUUGCACCUGUACCUCUUGGUCUUUGAAUA |
| | AAGCCUGAGUAGGAAGGCGG |

TABLE 4
<Seq ID 2> EPO-mRNA (850 bp:
5′-UTR + ORF + 3′-UTR + PolyA(120 nt)

| | |
|---|---|
| 5′- | AGGGAAAUAAGAGAGAAAAGAAGAGUAAGAAGAAAUAU |
| UTR | AAGAGCCACC |
| ORF | AUGGGCGUGCACGAGUGCCCCGCCUGGCUGUGGCUGCUG |
| | CUGAGCCUGCUGAGCCUGCCCCUGGGCCUGCCCGUGCUG |
| | GGCGCCCCCCCCCGGCUGAUCUGCGACAGCCGGGUGCUG |
| | GAGCGGUACCUGCUGGAGGCCAAGGAGGCCGAGAACAU |
| | CACCACCGGCUGCGCCGAGCACUGCAGCCUGAACGAGAA |
| | CAUCACCGUGCCCGACACCAAGGUGAACUUCUACGCCUG |
| | GAAGCGGAUGGAGGUGGGCCAGCAGGCCGUGGAGGUGU |
| | GGCAGGGCCUGGCCCUGCUGAGCGAGGCCGUGCUGCGGG |
| | GCCAGGCCCUGCUGGUGAACAGCAGCCAGCCCUGGGAGC |
| | CCCUGCAGCUGCACGUGGACAAGGCCGUGAGCGGCCUGC |
| | GGAGCCUGACCACCCUGCUGCGGGCCCUGGGCGCCCAGA |
| | AGGAGGCCAUCAGCCCCCCCGACGCCGCCAGCGCCGCCC |
| | CCCUGCGGACCAUCACCGCCGACACCUUCCGGAAGCUGU |
| | UCCGGGUGUACAGCAACUUCCUGCGGGGCAAGCUGAAGC |
| | UGUACACCGGCGAGGCCUGCCGGACCGGCGACCGGUGA |
| 3′- | UAAGCUGCCUUCUGCGGGGCUUGCCUUCUGGCCAUGCCC |
| UTR | UUCUUCUCUCCCUUGCACCUGUACCUCUUGGUCUUUGAA |
| | UAAAGCCUGAGUAGGAAGGCGG |

### <Synthesis of PolyA-Modified Fluc-mRNA>

Using the obtained Fluc-mRNA and oligonucleotides, PolyA-modified mRNA was synthesized by an enzymatic ligation reaction. The following components were mixed at the indicated concentrations: Fluc-mRNA (200 ng/μL), synthetic oligonucleotides (30 μM), ATP (10 mM NEB #M0204), PEG8000 (20% NEB #M0204), T4 RNA Ligase Reaction Buffer (1×NEB #M0204), SUPERase-In™ RNase Inhibitor (0.16 U/μL Thermo Fisher #AM2694), UltraPure DNase/RNase-Free Distilled Water (Thermo Fisher #10977015), and T4 RNA Ligase 1 (92 μg/mL). The mixture was incubated at 25° C. for 4 hours. mRNA was extracted from the resulting reaction mixture using AMPure XP (BECKMAN COULTER #A63881) and recovered into Amicon Ultra-0.5 mL 30K (Merck #UFC5030BK). After centrifugation (12000 rpm, 5 minutes) to concentrate the solution, 2 mM citrate buffer was added, and centrifugation (12000 rpm, 5 minutes) was performed again to exchange the buffer. This buffer exchange operation was repeated three times to obtain the purified PolyA-modified Fluc-mRNA (Table 5-1 and Table 5-2).

TABLE 5-1

| Sequence name | mRNA sequence | Structure |
|---|---|---|
| Fluc-6rA-idA | Seq. ID 1 | 5′-mRNA-AAAAAA-idA-3′ |
| Fluc-6rA-idG | Seq. ID 1 | 5′-mRNA-AAAAAA-idG-3′ |
| Fluc-6rA-idC | Seq. ID 1 | 5′-mRNA-AAAAAA-idC-3′ |
| Fluc-6rA-idT | Seq. ID 1 | 5′-mRNA-AAAAAA-idT-3′ |
| Fluc-6rA-iFA | Seq. ID 1 | 5′-mRNA-AAAAAA-iFA-3′ |
| Fluc-6rA-iFU | Seq. ID 1 | 5′-mRNA-AAAAAA-iFU-3′ |
| Fluc-6rA-iOMeA | Seq. ID 1 | 5′-mRNA-AAAAAA- |
| | | iOMeA-3′ |
| Fluc-6rA-iOMeU | Seq. ID 1 | 5′-mRNA-AAAAAA- |
| | | iOMeU-3′ |
| Fluc-6rA-imoeA | Seq. ID 1 | 5′-mRNA-AAAAAA- |
| | | imoeA-3′ |
| Fluc-6rA-imoeG | Seq. ID 1 | 5′-mRNA-AAAAAA- |
| | | imoeG-3′ |
| Fluc-6rA-imoemC | Seq. ID 1 | 5′-mRNA-AAAAAA- |
| | | imoemC-3′ |
| Fluc-6rA-imoeT | Seq. ID 1 | 5′-mRNA-AAAAAA- |
| | | imoe′T-3′ |
| Fluc-6rA-ps-imoeA | Seq. ID 1 | 5′-mRNA-AAAAAA- |
| | | ps-imoeA-3′ |
| Fluc-6rA-ilnaA | Seq. ID 1 | 5′-mRNA-AAAAAA-ilnaA-3′ |
| Fluc-6rA-ilnaT | Seq. ID 1 | 5′-mRNA-AAAAAA-ilnaT-3′ |
| Fluc-1rA-imocT | Seq. ID 1 | 5′-mRNA-A-imocT-3′ |
| Fluc-1rA-ps-imoeT | Seq. ID 1 | 5′-mRNA-A-ps-imoeT-3′ |
| Fluc-6rA-2′-3′-dT | Seq. ID 1 | 5′-mRNA-AAAAAA- |
| | | 2′-3′-dT-3′ |
| Fluc-6rA-ps2-idT | Seq. ID 1 | 5′-mRNA-AAAAAA- |
| | | ps2-idT-3′ |
| Fluc-6rA-idXT | Seq. ID 1 | 5′-mRNA-AAAAAA-idXT-3′ |
| Fluc-6rA-2′-3′-dXT | Seq. ID 1 | 5′-mRNA-AAAAAA- |
| | | 2′-3′-XdT-3′ |
| Fluc-6rA-ps-idXT | Seq. ID 1 | 5′-mRNA-AAAAAA- |
| | | ps-idXT-3′ |
| Fluc-5rA-dA-rp- | Seq. ID 1 | 5′-mRNA-AAAAA-dA- |
| ipmoA | | rp-ipmoA-3′ |
| Fluc-5rA-dA-sp- | Seq. ID 1 | 5′-mRNA-AAAAA-dA- |
| ipmoA | | sp-ipmoA-3′ |
| Fluc-1rG-imoeT | Seq. ID 1 | 5′-mRNA-G-imoeT-3′ |
| Fluc-1rC-imocT | Seq. ID 1 | 5′-mRNA-C-imocT-3′ |
| Fluc-1rU-imoeT | Seq. ID 1 | 5′-mRNA-U-imoeT-3′ |
| Fluc-1dT-imoeT | Seq. ID 1 | 5′-mRNA-dT-imoeT-3′ |
| Fluc-6rA-iC16A | Seq. ID 1 | 5′-mRNA-AAAAAA- |
| | | iC16A-3′ |
| Fluc-6rA-iallyIT | Seq. ID 1 | 5′-mRNA-AAAAAA- |
| | | iallylT-3′ |
| Fluc-6rA-imoeoeA | Seq. ID 1 | 5′-mRNA-AAAAAA- |
| | | imoeoeA-3′ |
| Fluc-2(1rA-imoeT) | Seq. ID 1 | 5′-mRNA-A-imoeT-A-imoeT3′ |
| Fluc-1rA-2imoeT | Seq. ID 1 | 5′-mRNA-A-imoeT-imoeT-3′ |
| Fluc-AGC-imoeT | Seq. ID 1 | 5′-mRNA-AGC-imoeT-3′ |
| Fluc-1rA-idmiPrT | Seq. ID 1 | 5′-mRNA-A-idmiPrT-3′ |
| Fluc-1rA-i2′-R-THFT | Seq. ID 1 | 5′-mRNA-A-i2′-R-THFT-3′ |
| Fluc-1rA-i2′-S-THFT | Seq. ID 1 | 5′-mRNA-A-i2′-S-THFT-3′ |

TABLE 5-2

| | | |
|---|---|---|
| Fluc-6rA-3′-2′-OBuA | Seq. ID 1 | 5′-mRNA-AAAAAA-3′-2′-OBuA- |
| | | 3′ |
| Fluc-1rA-iOMeA- | Seq. ID 1 | 5′-mRNA-A-iOMeA-N6-Bn-3′ |
| N6-Bn | | |
| Fluc-1rA-iBnT | Seq. ID 1 | 5′-mRNA-A-iBnT-3′ |
| Fluc-1rA-rp-imoeT | Seq. ID 1 | 5′-mRNA-A-ps(Rp)-imoeT-3′ |
| Fluc-1rA-sp-imoeT | Seq. ID 1 | 5′-mRNA-A-ps(Sp)-imoeT-3′ |
| Fluc-1rA-rp-rA- | Seq. ID 1 | 5′-mRNA-A-ps(Rp)-rA-imoeT-3′ |
| imoeT | | |
| Fluc-1rA-sp-rA- | Seq. ID 1 | 5′-mRNA-A-ps(Sp)-rA-imoeT-3′ |
| imoeT | | |
| Fluc-1rA-sp-rA-sp- | Seq. ID 1 | 5′-mRNA-A-ps(Sp)-rA-ps(Sp)-rA- |
| rA-imoeT | | imoeT-3′ |
| Fluc-1rA-sp-rA-sp- | Seq. ID 1 | 5′-mRNA-A-ps(Sp)-pA-ps(Sp)-rA- |
| rA-sp-rA-imoeT | | ps(Sp)-rA-imoeT-3′ |
| Fluc-6rA-imoeXT | Seq. ID 1 | 5′-mRNA-AAAAAA-imoeXT-3′ |

### <Synthesis of PolyA-Modified Epo-mRNA>

Using the obtained EPO-mRNA and oligonucleotides, PolyA-modified mRNA was synthesized by an enzymatic ligation reaction. The following components were mixed at the indicated concentrations: EPO-mRNA (200 ng/μL), synthetic oligonucleotides (30 μM), ATP (10 mM NEB #M0204), PEG8000 (20% NEB #M0204), T4 RNA Ligase Reaction Buffer (1×NEB #M0204), SUPERase-In™ RNase Inhibitor (0.16 U/μL Thermo Fisher #AM2694), UltraPure DNase/RNase-Free Distilled Water (Thermo Fisher #10977015), and T4 RNA Ligase 1 (92 μg/mL). The mixture was incubated at 25° C. for 4 hours. To the resulting reaction mixture, 8M LiCl (Sigma #L7026) was added to a final concentration of 2.7M, and the mixture was left standing at −20° C. for more than 1 hour. After centrifugation (4° C., 12000 rpm, 30 minutes), the supernatant was discarded. Then, 70% ethanol was added, and after centrifugation (4° C., 12000 rpm, 5 minutes), the supernatant was discarded again. The obtained precipitate was dissolved in 2 mM citrate buffer to obtain the crude PolyA-modified EPO-mRNA.

### <Purification of EPO-mRNA and PolyA-modified EPO-mRNA HPLC>

The obtained crude EPO-mRNA and PolyA-modified EPO-mRNA were each purified by reverse-phase ion-pair HPLC (HPLC: Shimadzu LC-20A series, Column: Agilent PLRP-S 4000A, 4.6×150 mm, S=10 μm, Mobile phase A: 0.1M TEAA, Mobile phase B: 0.1M TEAA containing 50% acetonitrile, Gradient conditions: Mobile phase B 19-34%/26 minutes, Flow rate: 1 mL/min, Column temperature: 75° C.). The HPLC-purified fractions were recovered into Amicon Ultra-15 mL 100K (Merck #UFC910024). After centrifugation (12000 rpm, 5 minutes) to concentrate the solution, 2 mM citrate buffer was added, and centrifugation (12000 rpm, 5 minutes) was performed again to exchange the buffer. This buffer exchange operation was repeated three times to obtain the HPLC-purified EPO-mRNA and PolyA-modified EPO-mRNA (Table 6).

TABLE 6

| Sequence name | mRNA sequence | Structure |
|---|---|---|
| EPO-mRNA | Seq. ID 2 | 5′-mRNA-3′ |
| EPO-6rA-idT | Seq. ID 2 | 5′-mRNA-AAAAAA-idT-3′ |
| EPO-6rA-imoeT | Seq. ID 2 | 5′-mRNA-AAAAAA-imoeT-3′ |

### <HPLC Analysis of EPO-mRNA and PolyA-modified EPO-mRNA>

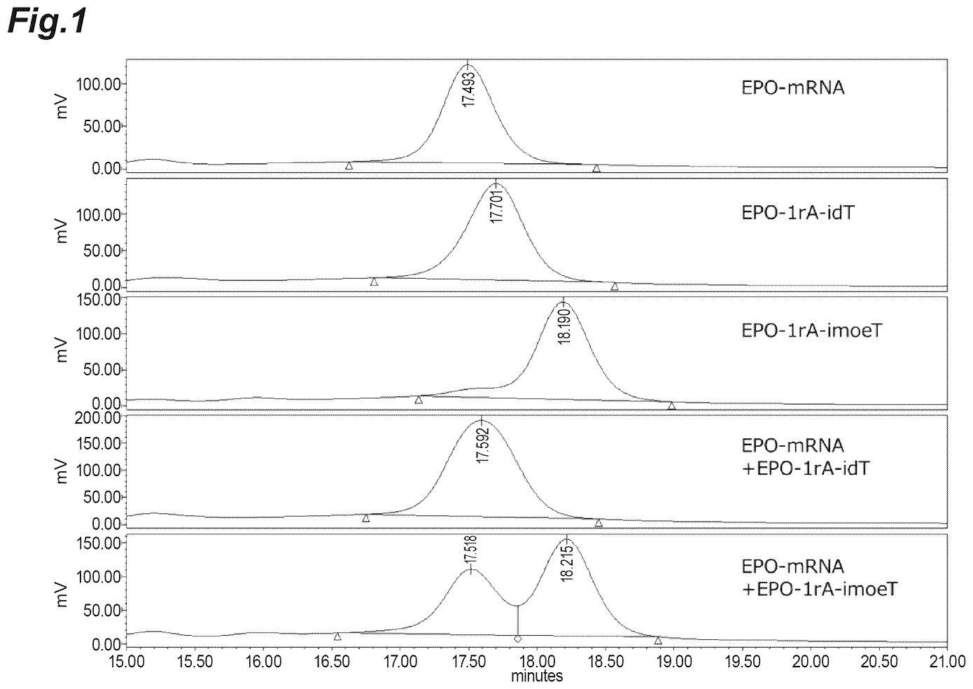

Using EPO-mRNA and oligonucleotides, PolyA-modified mRNA was synthesized by an enzymatic ligation reaction. The following components were mixed at the indicated concentrations: EPO-mRNA (200 ng/μL), synthetic oligonucleotides (30 μM), ATP (10 mM NEB #M0204), PEG8000 (20% NEB #M0204), T4 RNA Ligase Reaction Buffer (1×NEB #M0204), UltraPure DNase/RNase-Free Distilled Water (Thermo Fisher #10977015), and T4 RNA Ligase 1 (92 μg/mL). The mixture was incubated at 25° C. for 4 hours. mRNA was extracted from the resulting reaction mixture using AMPure XP (BECKMAN COULTER #A63881) and recovered into Amicon Ultra-0.5 mL 30K (Merck #UFC5030BK). After centrifugation (12000 rpm, 5 minutes) to concentrate the solution, 2 mM citrate buffer was added, and centrifugation (12000 rpm, 5 minutes) was performed again to exchange the buffer. This buffer exchange operation was repeated three times to obtain the purified PolyA-modified EPO-mRNA. EPO-mRNA and PolyA-modified EPO-mRNA were each analyzed by reverse-phase ion-pair HPLC (HPLC: Shimadzu LC-20A series, Column: YMC Triart Bio C4, 4.6×150 mm, S=5 μm, Mobile phase A: 0.1M TEAA, Mobile phase B: 0.1M TEAA containing 50% acetonitrile, Gradient conditions: Mobile phase B 19-28%/26 minutes, Flow rate: 1 mL/min, Column temperature: 75° C.). The HPLC analysis results and the retention times (RT) of the target substances are shown in FIG. 1 and Table 7.

TABLE 7

| | mRNA | | |
|---|---|---|---|
| Sequence name | sequence | Structure | RT (min) |
| EPO-mRNA | Seq. ID 2 | 5′-mRNA-3′ | 17.5 |
| EPO-1rA-idT | Seq. ID 2 | 5′-mRNA-A-idT-3′ | 17.7 |
| EPO-1rA-imocT | Scq. ID 2 | 5′-mRNA-A-imocT-3′ | 18.2 |

<PolyA Analysis of EPO mRNA>

The HPLC-purified EPO mRNA and PolyA-modified mRNA were enzymatically digested, and the structure of PolyA was analyzed. The following components were mixed at the indicated concentrations: mRNA (0.6 μg/μL), ultrapure water, RNase H Reaction Buffer (1×NEB #M0297), and RNase T1 (20 U/μL Thermo Fisher #10977015). The mixture was incubated at 37° C. for 3 hours. Free PolyA was purified from the resulting reaction mixture using Oligo d(T) 25 Magnetic Beads (40 μL NEB #S1419). The structure of the obtained free PolyA was analyzed using an LC-MS system (HPLC: Shimadzu Nexera series, MS: Thermo Q-Exactive, Column: Waters ACQUITY Premier Oligonucleotide C18 Column, 130 angstrom, 2.1×100 mm, S=1.7 μm, Mobile phase A: 8.6 mM TEA-100 mM HFIP, Mobile phase B: Methanol, Gradient conditions: Mobile phase B 10-25%/20 minutes, Flow rate: 0.2 mL/min, Column temperature: 60° C.). The MS analysis results of the free PolyA confirmed that modified nucleic acids were introduced at the PolyA termini (Table 8).

TABLE 8

| | | Calcd. MS | Observed MS |
|---|---|---|---|
| Sequence name | Free PolyA | [M − H]⁻ | [M − H]⁻ |
| EPO mRNA | 5′-A124-3′ | 40739.5 | 40735.3 |
| EPO-6rA-idT | 5′-A130-idT-3′ | 43017.9 | 43018.1 |
| EPO-6rA-imoeT | 5′-A130-imoeT-3′ | 43092.0 | 43086.8 |

### [Example 2] Time-Course of Luciferase Expression Using PolyA-Modified Fluc-mRNA In Vitro

The PolyA-modified Fluc-mRNA described in Table 5 and the transfection reagent Lipofectamine™ MessengerMAX™ (Invitrogen, Cat #LMRNA008) were diluted in Opti-MEM medium (Gibco, Cat #31985062) to prepare an mRNA/MessengerMAX mixture with a final concentration of 2.4 ng/μL nucleic acid and 0.95% MessengerMAX™. As a negative control, a mixture was prepared using PBS instead of nucleic acid. 20 μL of the mRNA/MessengerMAX mixture was dispensed into each well of a 96-well culture plate, and Hep3B cells (obtained from ATCC), a human liver cancer cell line, were seeded at 10,000 cells/80 μL/well and cultured under conditions of 37° C. and 5% CO₂. After 24 hours, 48 hours, and 72 hours, the supernatant was removed, and the Steady-Glo™ Luciferase Assay System (Promega, Cat #E2520) and CellTiter-Glo™ Luminescent Cell Viability Assay (Promega, Cat #G7572) were used to induce the enzymatic reaction according to the manufacturer's instructions, except that they were diluted 2-fold with PBS before addition. Absorbance was measured using Nivo (PerkinElmer). The measured data were corrected by setting the luciferase expression level of Fluc-mRNA (Fluc-mRNA without modified structure) at 24 hours post-administration to 100. The corrected measurement data and the analyzed results are shown in Table 9 and Table 10. In addition to the results shown in the Tables, the results of xylose-type modification (such as represented by formula (II′-AX) and formula (II′-A)) showed similar trends to that of ribose-type modification (such as represented by formula (II′-A) and formula (II′)).

TABLE 9

| | Relative Steady-Glo/Cell Titer-Glo | | | |
|---|---|---|---|---|
| | (Average, Fluc-mRNA 24 h = 100) | | | |
| Sequence name | 24 h | 48 h | 72 h | 72 h/24 h (%) |
| Fluc-mRNA | 100 | 26 | 5 | 5 |
| Fluc-6rA-idA | 165 | 83 | 30 | 18 |
| Fluc-6rA-idG | 143 | 80 | 26 | 18 |
| Fluc-6rA-idC | 131 | 50 | 15 | 11 |
| Fluc-6rA-idT | 149 | 68 | 21 | 14 |
| Fluc-6rA-iFA | 154 | 76 | 24 | 16 |
| Fluc-6rA-iFU | 150 | 69 | 23 | 15 |
| Fluc-6rA-iOMeA | 161 | 69 | 19 | 12 |
| Fluc-6rA-iOMeU | 204 | 111 | 46 | 23 |
| Fluc-6rA-imoeA | 127 | 63 | 25 | 20 |
| Fluc-6rA-imoeG | 141 | 87 | 37 | 26 |
| Fluc-6rA-imoemC | 135 | 80 | 34 | 25 |
| Fluc-6rA-imoeT | 154 | 97 | 45 | 29 |
| Fluc-6rA-ps-imoeA | 110 | 52 | 22 | 20 |
| Fluc-6rA-ilnaA | 77 | 22 | 5 | 6 |
| Fluc-6rA-ilnaT | 79 | 24 | 5 | 6 |
| Fluc-1rA-imoeT | 169 | 114 | 61 | 36 |
| Fluc-1rA-ps-imoeT | 127 | 66 | 22 | 17 |

TABLE 10

| | Relative Steady-Glo/Cell Titer-Glo | | | |
|---|---|---|---|---|
| | (Average, Fluc-mRNA 24 h = 100) | | | |
| Sequence name | 24 h | 48 h | 72 h | 72 h/24 h (%) |
| Fluc-mRNA | 100 | 25 | 5 | 5 |
| Fluc-6rA-2′-3′-dT | 88 | 24 | 4 | 5 |
| Fluc-6rA-ps2-idT | 83 | 24 | 6 | 7 |
| Fluc-6rA-idXT | 161 | 63 | 16 | 10 |
| Fluc-6rA-2′-3′-dXT | 153 | 67 | 16 | 10 |
| Fluc-6rA-ps-idXT | 185 | 90 | 31 | 17 |
| Fluc-5rA-dA-rp- | 96 | 15 | 3 | 3 |
| ipmoA | | | | |
| Fluc-5rA-dA-sp- | 105 | 36 | 5 | 5 |
| ipmoA | | | | |
| Fluc-1rG-imoeT | 107 | 39 | 6 | 6 |
| Fluc-1rC-imoeT | 171 | 112 | 36 | 21 |
| Fluc-1rU-imoeT | 154 | 86 | 25 | 16 |
| Fluc-1dT-imoeT | 55 | 25 | 7 | 13 |
| Fluc-6rA-iC16A | 134 | 82 | 30 | 22 |
| Fluc-6rA-iallylT | 168 | 115 | 44 | 26 |
| Fluc-6rA-imoeoeA | 168 | 91 | 26 | 15 |
| Fluc-2(1rA-imoeT) | 62 | 22 | 5 | 8 |
| Fluc-1rA-2imoeT | 47 | 17 | 4 | 9 |
| Fluc-AGC-imoeT | 105 | 53 | 12 | 11 |
| Fluc-1rA-idmiPrT | 162 | 125 | 50 | 31 |
| Fluc-1rA-i2′-R- | 147 | 125 | 45 | 31 |
| THFT | | | | |
| Fluc-1rA-i2′-S- | 151 | 125 | 50 | 33 |
| THFT | | | | |

### [Example 3] Time-Course of Erythropoietin Expression Using PolyA-Modified EPO-mRNA In Vitro

The mRNA described in Table 6 and the transfection reagent Lipofectamine™ MessengerMAX™ (Invitrogen, Cat #LMRNA008) were diluted in Opti-MEM medium (Gibco, Cat #31985062) to prepare an mRNA/MessengerMAX mixture with a final concentration of 2.4 ng/μL nucleic acid and 0.95% MessengerMAX™. As a negative control, a mixture was prepared using PBS instead of nucleic acid. 20 μL of the mRNA/MessengerMAX mixture was dispensed into each well of a 96-well culture plate, and Hep3B cells (obtained 10 from ATCC), a human liver cancer cell line, were seeded at 10,000 cells/80 μL/well and cultured under conditions of 37° C. and 5% CO₂. After 24 hours, 48 hours, 72 hours, 96 hours, and 168 hours, the Erythropoietin in the culture supernatant was immobilized and labeled according to the manufacturer's protocol using the Quantikine™ ELISA Human Erythropoietin Immunoassay (R&D, Cat #DEPRU0). Absorbance was measured using Envision E2105™ (PerkinElmer), and the concentration of Erythropoietin was calculated. The results are shown in Table 11.

TABLE 11

| | EPO-mRNA | EPO-6rA-idT | EPO-6rA-imoeT |
|---|---|---|---|
| Time | (mIU/mL, Average) | (mIU/mL, Average) | (mIU/mL, Average) |
| 24 h | 114816 | 123593 | 145063 |
| 48 h | 143372 | 227509 | 295152 |
| 72 h | 164956 | 240430 | 438563 |
| 96 h | 150523 | 247585 | 462389 |
| 168 h | 164831 | 216996 | 497434 |

### [Example 4] Time-Dependent Change of the Expression of Erythropoietin Expressed by Using PolyA-Modified EPO-mRNA In Vivo

### <Preparation of Lipid Nanoparticles (LNP)>

The PolyA-modified EPO-mRNA described in Table 6 was dissolved in 50 mM sodium citrate (pH 3.5) to prepare an mRNA dilution. 2-{9-Oxo-9-[(3-pentyloctyl)oxy]nonyl}dodecyl 1-methylpiperidine-4-carboxylate (TS202), DSPC (Cas #816-94-4), Cholesterol (Cas #57-88-5), and MPEG2000-DMG (Cas #1397695-86-1) were dissolved in ethanol at a molar ratio of 50/10/38.5/1.5 to prepare a lipid solution. The mRNA dilution and lipid solution were mixed at a flow rate ratio of 3:1 to achieve an mRNA-to-lipid weight ratio of 0.056:1, thereby obtaining Lipid Nanoparticles (LNP). The aqueous solution containing the obtained LNP was dialyzed using Float-A-Lyzer G2 (SPECTRUM, 100K MWCO) to replace the external solution with PBS, and then further replaced with a sucrose solution. After concentrating the solution, it was sterilized by filtration, and the formulation quality was evaluated. The mRNA concentration and encapsulation efficiency were measured using Quant-iT RiboGreen RNA Reagent (Invitrogen, Cat #R11491) (the mRNA concentration measured after dilution with RNase-Free Water was considered as the mRNA present in the external solution of LNP, and the mRNA concentration measured after dilution with 1% Triton X-100 was considered as the total mRNA concentration in the formulation to calculate the encapsulation efficiency). The average particle size (Z-average) was measured using a particle size analyzer (Malvern, Zetasizer Nano ZS). The results of the formulation quality evaluation of the prepared LNP are shown in Table 12.

TABLE 12

| | | Average particle | | Encapsulation |
|---|---|---|---|---|
| LNP ID | mRNA | size (nm) | PDI | efficiency (%) |
| #01 | EPO-mRNA | 90 | 0.05 | 95% |
| #02 | EPO-6rA-idT | 89 | 0.07 | 93% |
| #03 | EPO-6rA-imoeT | 88 | 0.02 | 94% |

### <Time-Dependent Change of the Expression of Erythropoietin in Vivo>

Saline or the LNP shown in Table 12 was intravenously administered to BALB/c mice (female, 5 weeks old, n=3 per group) via the tail vein at a dose of 0.2 mg/kg mRNA. Blood samples were collected from the tail vein at 6 hours, 24 hours, 48 hours, 72 hours, and 96 hours post-administration, and from the abdominal aorta under general anesthesia at 168 hours post-administration, using heparin sodium (Yoshindo Inc., Cat #AY). The Erythropoietin in the plasma was immobilized and labeled according to the manufacturer's protocol using the Quantikine™ ELISA Human Erythropoietin Immunoassay (R&D, Cat #DEPRU0). Absorbance was measured using Spectramax™ iD5 (Molecular Devices, LLC), and the concentration was calculated. The results are shown in Table 13.

TABLE 13

| | EPO-mRNA | EPO-6rA-idT | BPO-6rA-imoeT |
|---|---|---|---|
| Time | (mIU/mL, Average) | (mIU/mL, Average) | (mIU/mL, Average) |
| 6 h | 164607 | 117720 | 142263 |
| 24 h | 124134 | 109816 | 125770 |
| 48 h | 7381 | 36720 | 72237 |
| 72 h | 98 | 15820 | 41147 |
| 96 h | 13 | 7448 | 25514 |
| 168 h | 0 | 319 | 1999 |

What is claimed is:

1. A polynucleotide represented by the following formula (I): 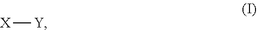
wherein Y is represented by the following formula (II): 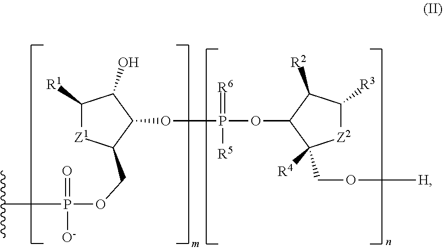
wherein
the wavy line represents a bond to X,
R¹ and R³ each independently represent a nucleobase, wherein if Y has two or more R¹, then each of the two or more R¹ may independently represent a nucleobase; and wherein if Y has two or more R³, then each of the two or more R³ may independently represent a nucleobase,
R² represents a group obtained by removing one hydrogen atom from a hydroxyl group of a linear or cyclic oligoalkylene glycol, a group in which one hydrogen atom on a carbon atom of a cyclic ether is replaced by an oxygen atom, an alkoxy, an optionally substituted benzyloxy, a halogen, a hydroxy, an alkenyl, or an alkyl; or together with R⁴, represents a group represented by —O—R¹⁰—, wherein R¹⁰ represents an alkylene, and wherein the oxygen atom in the group forms a single bond with the carbon atom to which R² is bonded in formula (II),
R⁴ represents H or, together with R², represents the group represented by —O—R¹⁰—,
R⁵ represents O⁻, S⁻, BH₃⁻, Se⁻, or dialkylamino,
R⁶ represents O, S, or Se,
Z¹ and Z² each independently represent O, S, Se, NR¹², or CR¹³R¹⁴, wherein R¹², R¹³, and R¹⁴ each independently represent an alkyl or alkenyl,
and m and n each independently represent an integer of 1 to 50,
wherein X is a polyribonucleotide comprising a 5′-cap structure at the 5′-end and a polyadenyl region at the 3′-end, of which a hydroxyl group bonded to the 3′-carbon of a nucleoside present at a terminal of the polyadenyl region forms a phosphodiester bond together with the terminal of Y.

2. The polynucleotide according to claim 1, wherein n is 1.

3. The polynucleotide according to claim 1, wherein R⁵ is O⁻ or S⁻, and R⁶ is O.

4. The polynucleotide according to claim 1, wherein R⁵ is O, and R⁶ is O.

5. The polynucleotide according to claim 1, wherein the nucleobase is thymine, uracil, cytosine, adenine, guanine, 5-methylcytosine, 6-methyladenine, 6-benzyladenine, 1-methyluracil, 5-hydroxymethylcytosine, 2-thiouracil, or hypoxanthine.

6. The polynucleotide according to claim 1, wherein R¹ is adenine, guanine, cytosine, thymine, or uracil, and R³ is thymine, uracil, cytosine, adenine, guanine, or 5-methylcytosine.

7. The polynucleotide according to claim 1, wherein R¹ is adenine, cytosine, or uracil, and R³ is thymine, uracil, cytosine, adenine, guanine, or 5-methylcytosine.

8. The polynucleotide according to claim 1, wherein R¹ is adenine, and R³ is thymine.

9. The polynucleotide according to claim 1, wherein R² represents a group obtained by removing one hydrogen atom from a hydroxyl group of a linear or cyclic oligoalkylene glycol having 1 to 15 carbon atoms and 1 to 10 oxygen atoms, a group in which one hydrogen atom on a carbon atom of a cyclic ether having 1 to 15 carbon atoms and 1 to 10 oxygen atoms is replaced by an oxygen atom, a C₁-C₂₀ alkoxy, a benzyloxy, fluoro, a C₂-C₁₀ alkenyl, or a C₁-C₁₀ alkyl, and R⁴ represents H.

10. The polynucleotide according to claim 1, wherein R² represents a group represented by CH₃—(O—CH₂—CH₂—)ₚO— or (CH₃O(—CH₂—CH₂—O—)_{w}CH₂—)₂CH—O—, in which p represents an integer of 1 to 2 and w represents an integer of 0 to 2; a group in which one hydrogen atom on a carbon atom of tetrahydrofuran or tetrahydropyran is replaced by an oxygen atom; a C₁-C₁₆ alkoxy; a benzyloxy; a C₂-C₅ alkenyl; or a C₁-C₅alkyl, and R⁴ represents H.

11. The polynucleotide according to claim 1, wherein R² is selected from the group consisting of methoxy-ethyleneoxy, methoxy, a benzyloxy, allyl, and a group represented by the following formulas: 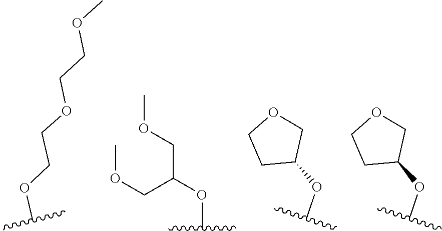
and R⁴ represents H.

12. The polynucleotide according to claim 1, wherein R² represents methoxy-ethyleneoxy, and R⁴ represents H.

13. The polynucleotide according to claim 1, wherein Y is represented by the following formula (VI): 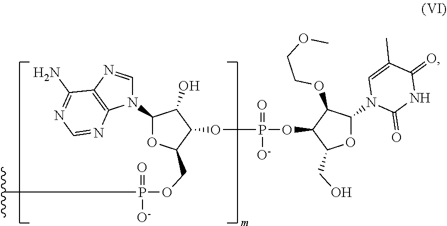
wherein the wavy line represents a bond to X, and m represents an integer of 1 to 20
wherein X is a polynucleotide comprising a 5′-cap structure at the 5′-end and of which a hydroxyl group bonded to the 3′-carbon of a nucleoside present at the 3′-end forms a phosphodiester bond together with the terminal of Y.

14. A producing method of the polynucleotide according to claim 1, comprising contacting a polynucleotide represented by the following formula (VII): 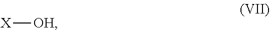
wherein, X is the same as X in formula (I), and OH represents a hydroxyl group bonded to the 3′-carbon of a nucleoside present at a terminal of the polyadenyl region,
with an oligonucleotide represented by the following formula (VIII): 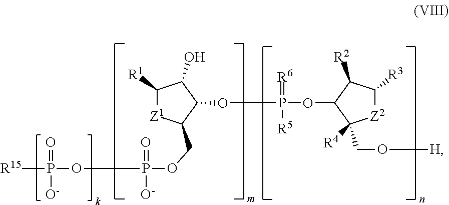
wherein
R¹ to R⁶, Z¹, Z², m, and n are the same as those in formula (II),
R¹⁵ represents O⁻ or a group obtained by removing one hydrogen atom from a hydroxyl group of the hydroxymethyl group at the 5′-position of adenosine,
and k represents an integer of 0 to 2,
in the presence of RNA ligase or poly(A) polymerase to obtain the polynucleotide represented by formula (I).

15. The producing method according to claim 14, comprising contacting a polynucleotide represented by the following formula (VII):
X—OH (VII)
wherein, X is the same as X in formula (I), and OH represents a hydroxyl group bonded to the 3′-carbon of a nucleoside present at a terminal of the polyadenyl region,
with an oligonucleotide represented by the following formula (XII): 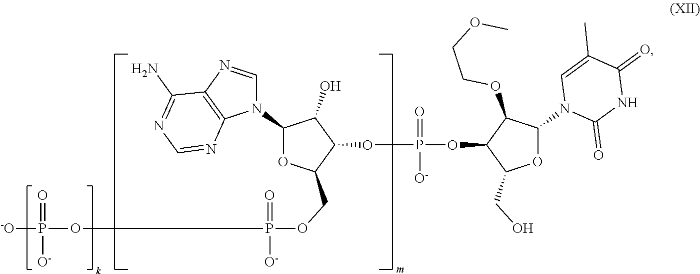
wherein
m is the same as those in formula (VI),
and k represents an integer of 0 to 2,
in the presence of RNA ligase or poly(A) polymerase to obtain the polynucleotide represented by formula (I).

16. The producing method according to claim 14, wherein m represents an integer of 1 to 20, 1 to 15, 1 to 12, 1 to 10, 1 to 9, 1 to 8, 1 to 7, or 1 to 6.

17. The producing method according to claim 14, wherein the RNA ligase or poly(A) polymerase is RNA ligase.
